(19) United States
(12) Patent Application Publication
(10) Pub. No.: US 2015/0004100 A1
(43) Pub. Date: Jan. 1, 2015

(54) HETEROCYCLIC COMPOUNDS AS IMAGING PROBES OF TAU PATHOLOGY

(71) Applicant: GE HEALTHCARE LIMITED, BUCKINGHAMSHIRE (GB)

(72) Inventors: Clare Jones, Amersham (GB); Matthias Eberhard Glaser, Amersham (GB); Duncan Wynn, Amersham (GB); James Nairne, Amersham (GB); Umamaheshwar P. Mokkapati, Bangalore (IN); Ian Martin Newington, Amersham (GB); Chitralekha Rangswamy, Bangalore (IN); Jinto Jose, Bangalore (IN); Saga Johansson, Amersham (GB)

(73) Assignee: GE HEALTHCARE LIMITED, BUCKINGHAMSHIRE (GB)

(21) Appl. No.: 14/365,652

(22) PCT Filed: Dec. 13, 2012
(86) PCT No.: PCT/US2012/069367

§ 371 Date: Jun. 16, 2014

Related U.S. Application Data
(60) Provisional application No. 61/570,915, filed on Dec. 15, 2011.

(51) Int. Cl. A61K 51/04; C07B 59/00; A61K 49/00; G01N 33/68

(52) U.S. Cl. CPC A61K 51/0459; G01N 33/6896; C07B 59/002; A61K 49/0052; G01N 2800/2821
USPC 424/1.89; 544/235; 435/7.1; 424/9.6

(57) ABSTRACT

Pyridazinone compounds of Formula I: (I) wherein: R′ is alkyl or Ar, optionally substituted with at least one alkyl, halogen, hydroxyl, alkoxy, haloalkoxy, acid, ester, amino, nitro, amide, or alkoxyhalo; 2 R is independently alkyi, alkynyl, ester, amino, amide, acid, aryl, heteroaryl, aminoalkyl, —C(=0)alkyl, —C(=0)aryl, —C(=0)heteroaryl, —C(=0)heterocycloalkyl, —C(=0)heterocycloalkylAr, —C(=0)(CH₂)ₙhalo, —C(═O)(CH₂)nheterocyclyl, or —SĈAr, optionally substituted with at least one alkyi, alkylhalo, halogen, nitro, aryl, heteroaryl, or heteroaryl(CH₂)nhalo; R³ and R⁴ are independently hydrogen, alkyi, alkenyl, alkynyl, aryl, heteroaryl; Ar is an aryl, heteroaryl, cycloalkyl, heterocycloalkyl group; n is an integer from 0-10; or a radiolabeled derivative thereof. The compounds are useful as imaging probes of Tau pathology in Alzheimer's disease are described. Compositions and methods of making such compounds are also described.

### TECHNICAL FIELD OF THE INVENTION

The present invention relates to radiolabeled pyridazinone compounds, compositions thereof, methods of making such compounds and their use as imaging probes of Tau pathology especially as it relates to Alzheimer's Disease. Compounds of the present invention may be used for Positron Emission Tomography (PET) or Single Photon Emission Computed Tomography (SPECT) imaging.

### DESCRIPTION OF RELATED ART

Alzheimer's disease (AD) is the most common cause of dementia in the elderly. It is definitively diagnosed and staged on the basis of post-mortem neuropathology. The pathological hallmark of AD is a substantial neuronal loss accompanied by deposition of amyloid plaques and neurofibrillary tangles (NFTs).

NFTs consist of filamentous aggregates composed of microtubule-associated protein tau. Much of the literature suggests that tau aggregates (NFTs) or NFT formation correlate more closely with AD progression than amyloid plaques (Braak, H. et al., Neuropathological Staging of Alzheimer-related Changes. Acta Neuropathologica, 82, 239-259, 1991). The tau aggregates or neurofibrillary lesions reportedly appear in areas (deep temporal lobe) decades before neocortical amyloid deposition and signs of dementia can be detected. The tau lesions occur before the presentation of clinical symptoms or signs of dementia and correlate with the severity of dementia. These attributes make tau aggregates a potentially superior approach for the early diagnosis of AD. Hence in vivo detection of these lesions or NFTs would prove useful for diagnosis of AD and for tracking disease progression.

One of the challenges in discovering NFT imaging probes is the selectivity for other protein aggregates (such as amyloid plaques) containing a cross beta-sheet conformation. Kudo et al. have recently screened compounds for selectivity to aggregated tau over amyloid in vitro. BF-170 and BF-158 were described as being ˜threefold selective for tau aggregates over Aβ1-42 amyloid:

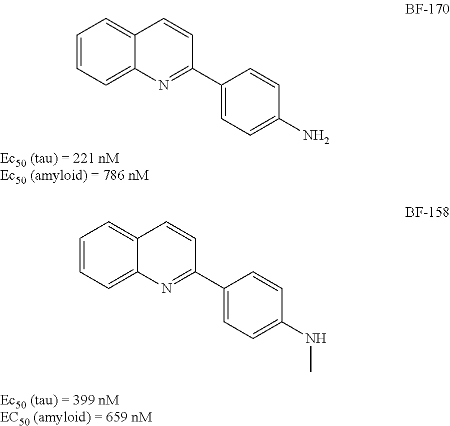 [SMILES: Nc1ccc(-c2ccc3ccccc3n2)cc1 ; CNc1ccc(-c2ccc3ccccc3n2)cc1]

(Kudo, Y., et al., J. Neuroscience, 2005, 25(47):10857-10862). These compounds and other quinoline derivatives are also described in US 2005/0009865, now U.S. Pat. No. 7,118,730, as diagnostic probes for the imaging diagnosis of diseases in which tau protein accumulates. The probes can be labeled with a radionuclide.

WO2011/037985 describes aminothienopyridazine inhibitors of tau assembly.

However there still exist a need in the art for other compounds that can be used as imaging agents for NFTs. The present invention described below answers such a need.

### BRIEF DESCRIPTION OF THE DRAWINGS

FIG. 1 is a preparative HPLC chromatogram showing product 37* eluting at 12.8 min (top: UV channel at 254 nm, bottom: radioactivity channel).

FIG. 2 is an analytical HPLC chromatogram showing product 37* eluting at 7.2 min (top: UV channel at 254 nm, bottom: radioactivity channel).

FIG. 3 is an analytical HPLC chromatogram showing product 38* eluting at 6.8 min (top: UV channel at 254 nm, bottom: radioactivity channel).

FIG. 4 is an analytical HPLC chromatogram showing product 38* eluting at 6.8 min and spiked standard compound 19F-38 at 6.7 min (top: UV channel at 254 nm, bottom: radioactivity channel).

FIG. 5 depicts Histology of human AD tissue sections. Numerous tau+ NFTs (A-B, arrow) and Aβ+ plaques (E-F, arrow) were observed in AD tissue sections. In addition, NFTs (C, arrow) and neuritic plaques (D, arrow) were also observed in tissue sections labelled with Gallyas silver stain (E-F). 10×: A, C, E, scale bar 100 μM; 20×: B, D, F, scale bar: A, 25 μM.

FIG. 6 depicts Binding of novel compounds to NFTs and plaques in AD tissue. 38 (A, B) binds to both NFTs (A) and plaques (B) at high test concentrations. Similary, 105 (C, D) also binds to NFTs (C, D) and plaques (D) at high test concentrations. At lower concentrations, both compounds binds preferentially to NFTs (Table 3). Arrows=NFTs, *=plaques. A and C: 40×, B and D: 20×.

### SUMMARY OF THE INVENTION

The present invention provides novel pyridazinone compounds for use as imaging probes of Tau pathology in Alzheimer's disease. The compounds of the inventions may be radiolabeled such that they may be used for in vitro and in vivo imaging purposes.

The present invention provides a compound of Formula I:

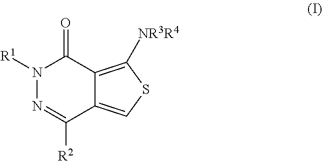

wherein:
R¹ is alkyl or Ar, optionally substituted with at least one alkyl, halogen, hydroxyl, alkoxy, haloalkoxy, acid, ester, amino, nitro, amide, or alkoxyhalo;
R² is independently hydrogen, alkyl, alkynyl, ester, amino, amide, acid, aryl, heteroaryl, aminoalkyl, —C(═O)alkyl, —C(═O)aryl, —C(═O)heteroaryl, —C(═O)heterocycloalkyl, —C(═O)heterocycloalkylAr, —C(═O)(CH₂)ₚhalo, —C(═O)(CH₂)ₚheterocyclyl, or —SO₂Ar, optionally substituted with at least one alkyl, alkylhalo, halogen, nitro, aryl, heteroaryl, or heteroaryl(CH₂)ₚhalo;
R³ and R⁴ are independently hydrogen, alkyl, alkenyl, alkynyl, acyl, aryl, heteroaryl;
Ar is an aryl, heteroaryl, cycloalkyl, heterocycloalkyl group;
p is an integer from 0-10; preferably, 0-5; more preferably, 0-3;
or a radiolabelled derivative thereof.

The present invention further provides a pharmaceutical composition comprising a compound of Formula (I) or a radiolabelled derivative thereof and a pharmaceutically acceptable carrier or excipient.

The present invention further provides a method of making a compound of Formula (I) or a radiolabelled derivative thereof.

The present invention further provides a method of imaging using a radiolabelled derivative of a compound of Formula (I) or a pharmaceutical composition thereof.

The present invention further provides a method of detecting tau aggregates in vitro and/or vivo using a radiolabelled derivative of a compound of Formula (I) or a pharmaceutical composition thereof.

### DETAILED DESCRIPTION OF THE INVENTION

The present invention provides pyridazinone compounds of Formula (I) as described herein.

In a preferred embodiment of the invention, a compound of Formula (I), as described above, is provided wherein Ar is:

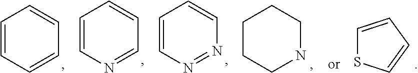

In a preferred embodiment of the invention, a compound of Formula (I), as described above, is provided wherein Ar of R¹ is:

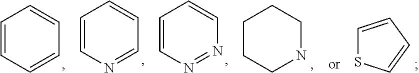

preferably

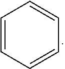

The present invention provides a compound of Formula (I) having Formula (Ia):

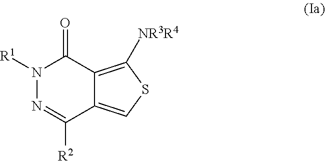

wherein:
R¹ is alkyl or Ar, optionally substituted with at least one alkyl, halogen, hydroxyl, alkoxy, haloalkoxy, acid, ester, amino, nitro, amide, or alkoxyhalo;
R² is independently hydrogen, alkyl, alkynyl, ester, amino, amide, acid, aryl, heteroaryl, aminoalkyl, —C(═O)alkyl, —C(═O)aryl, —C(═O)heteroaryl, —C(═O)heterocycloalkyl, —C(═O)heterocycloalkylAr, —C(═O)(CH₂)ₚhalo, —C(═O)(CH₂)ₚheterocyclyl, or —SO₂Ar, optionally substituted with at least one alkyl, alkylhalo, halogen, nitro, aryl, heteroaryl, or heteroaryl(CH₂)ₚhalo;
R³ and R⁴ are independently hydrogen, alkyl, alkenyl, alkynyl, acyl, aryl, heteroaryl;
Ar is an aryl, heteroaryl, cycloalkyl, heterocycloalkyl group;
p is an integer from 0-10; preferably, 0-5; more preferably, 0-3;
or a radiolabelled derivative thereof.

The present invention provides a compound of Formula (I) having Formula (Ib):

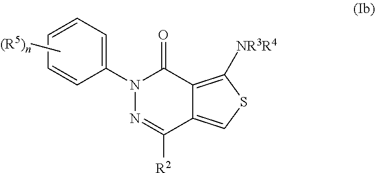

wherein:

R², R³, and R⁴ are each as defined herein for a compound of Formula (I);

R⁵ is hydrogen, alkyl, halogen, hydroxyl, alkoxy, haloalkoxy, acid, ester, amino, nitro, or amide; and

n is an integer from 0-5; or a radiolabelled derivative thereof. The present invention provides a compound of Formula (I) having Formula (Ic):

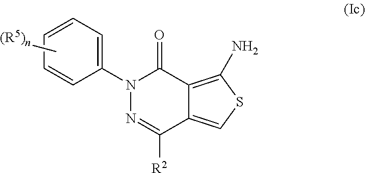

wherein:

R² is as defined herein for a compound of Formula (I);

R⁵ is hydrogen, alkyl, halogen, hydroxyl, alkoxy, haloalkoxy, acid, ester, amino, nitro, or amide; and

n is an integer from 0-5;

or a radiolabelled derivative thereof.
The present invention provides a compound of Formula (I) having Formula (Ida), (Idb) or (Idc):

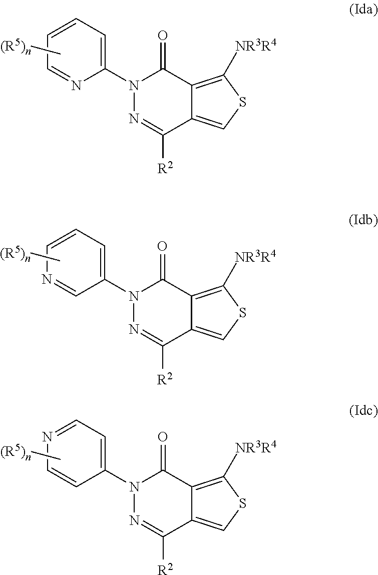

wherein:

R², R³, and R⁴ are each as defined herein for a compound of Formula (I);

R⁵ is hydrogen, alkyl, halogen, hydroxyl, alkoxy, haloalkoxy, acid, ester, amino, nitro, or amide; and

n is an integer from 0-5;

or a radiolabelled derivative thereof.
The present invention provides a compound of Formula (I) having Formula (Iea), (Ieb) or (Iec):

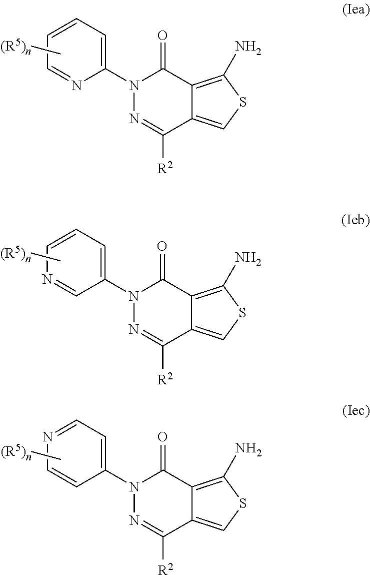

wherein:

R² is as defined herein for a compound of Formula (I);

R⁵ is hydrogen, alkyl, halogen, hydroxyl, alkoxy, haloalkoxy, acid, ester, amino, nitro, or amide; and

n is an integer from 0-5;

or a radiolabelled derivative thereof.

The present invention provides a compound of Formula (I) having Formula (If):

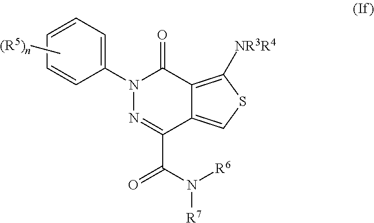

wherein:

R³ and R⁴ are each as defined herein for a compound of Formula (I);

R⁵ is hydrogen, alkyl, halogen, hydroxyl, alkoxy, haloalkoxy, acid, ester, amino, nitro, or amide;

n is an integer from 0-5;

R⁶ and R⁷ are independently hydrogen, alkyl, or alkynyl, or when taken together with the nitrogen to which they are attached form a heteroaryl or heterocycloalkyl optionally substituted with at least one alkyl, alkylhalo, halogen, hydroxyl, nitro, aryl, heterocycloalkyl, heteroaryl, or heteroarylhalo;

or a radiolabelled derivative thereof.

In one or more embodiments of the invention, the compound of Formula (I) is:

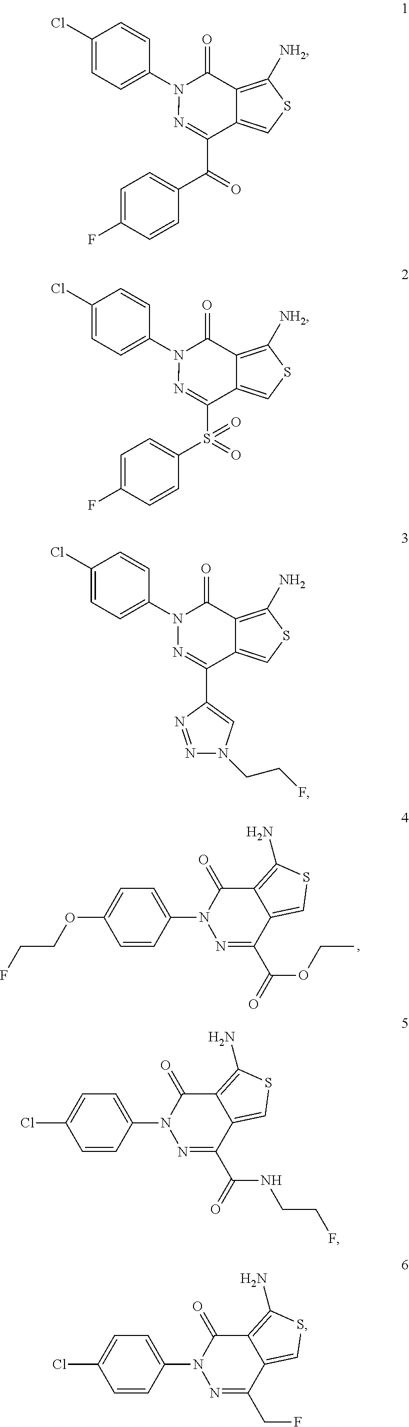

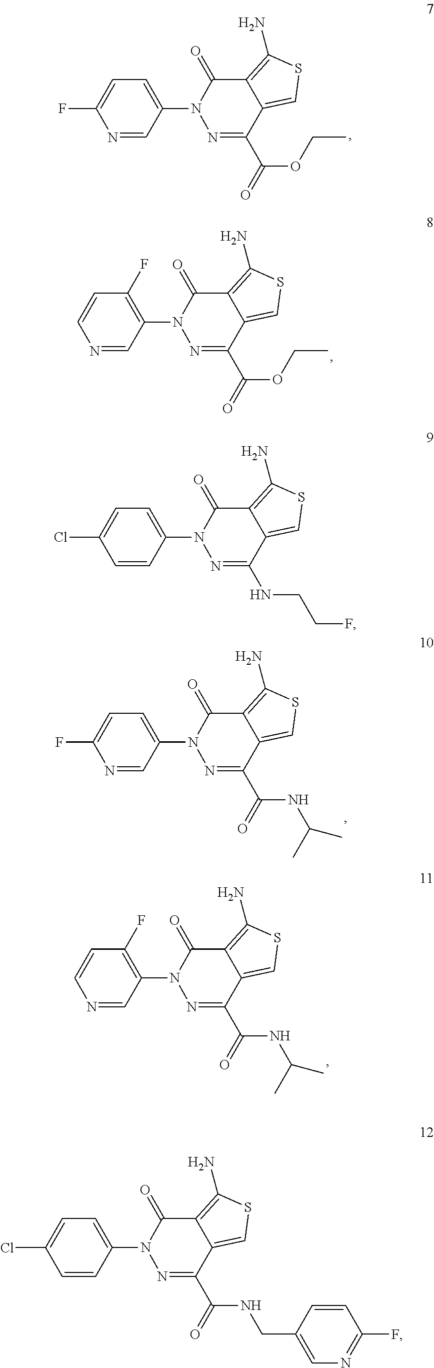

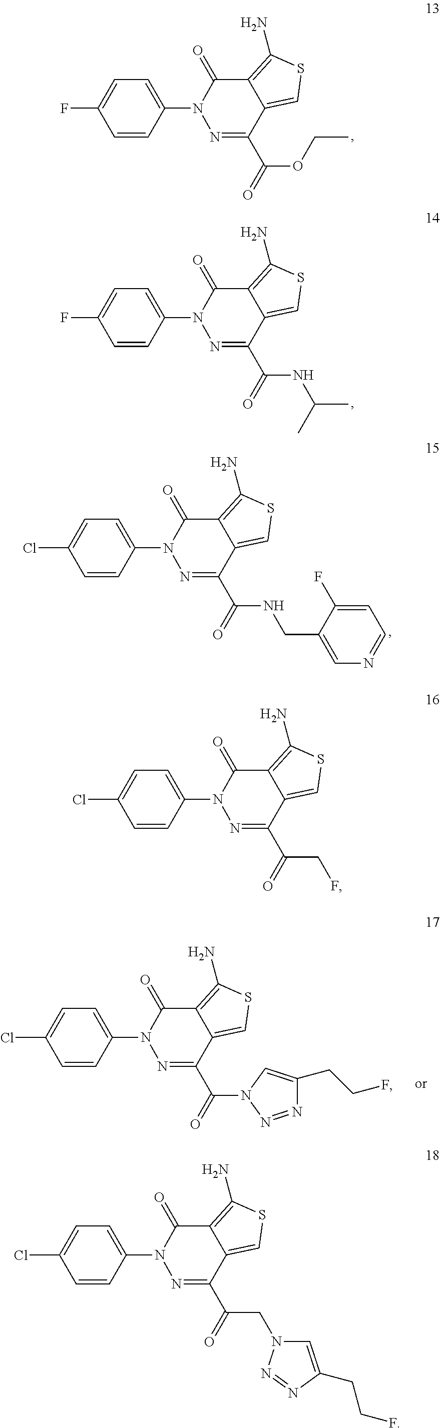

In one or more embodiments of the invention, the compound of Formula (I) is:

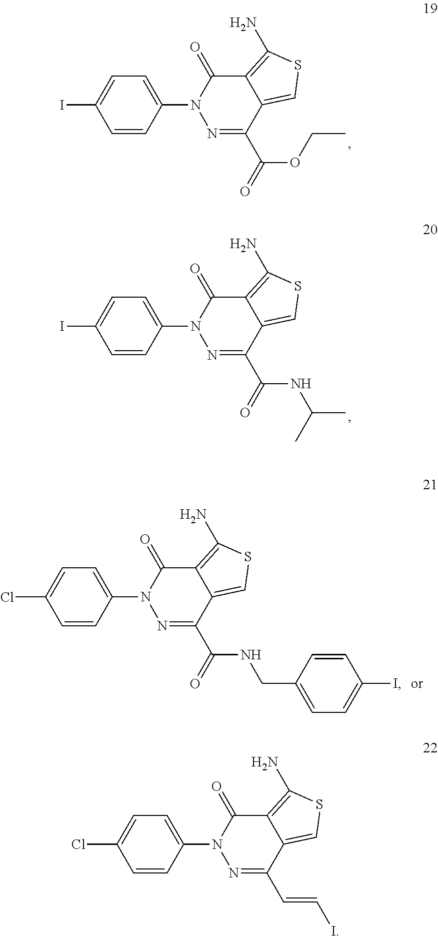 [SMILES: CCOC(=O)c1nn(-c2ccc(I)cc2)c(=O)c2c(N)scc12 ; CC(C)NC(=O)c1nn(-c2ccc(I)cc2)c(=O)c2c(N)scc12 ; Nc1scc2c(C(=O)NCc3ccc(I)cc3)nn(-c3ccc(Cl)cc3)c(=O)c12 ; Nc1scc2c(/C=C/I)nn(-c3ccc(Cl)cc3)c(=O)c12]

In one or more embodiments of the invention, the compound of Formula (I) is

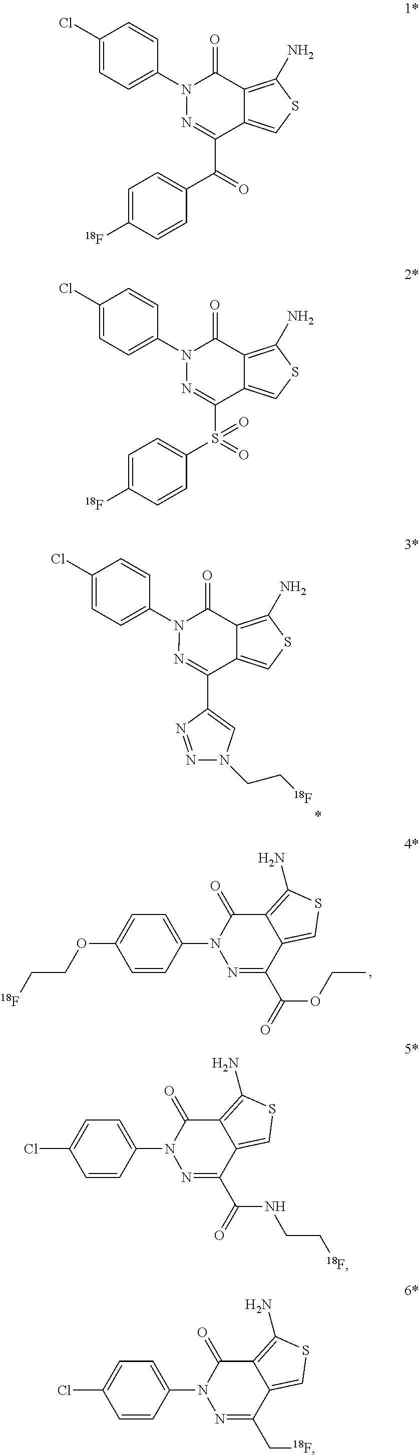

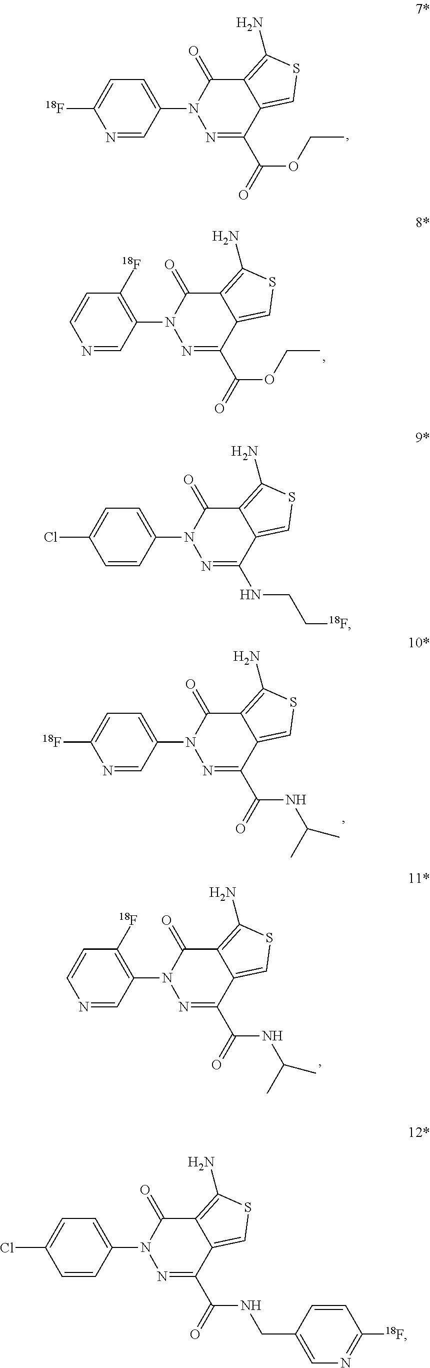

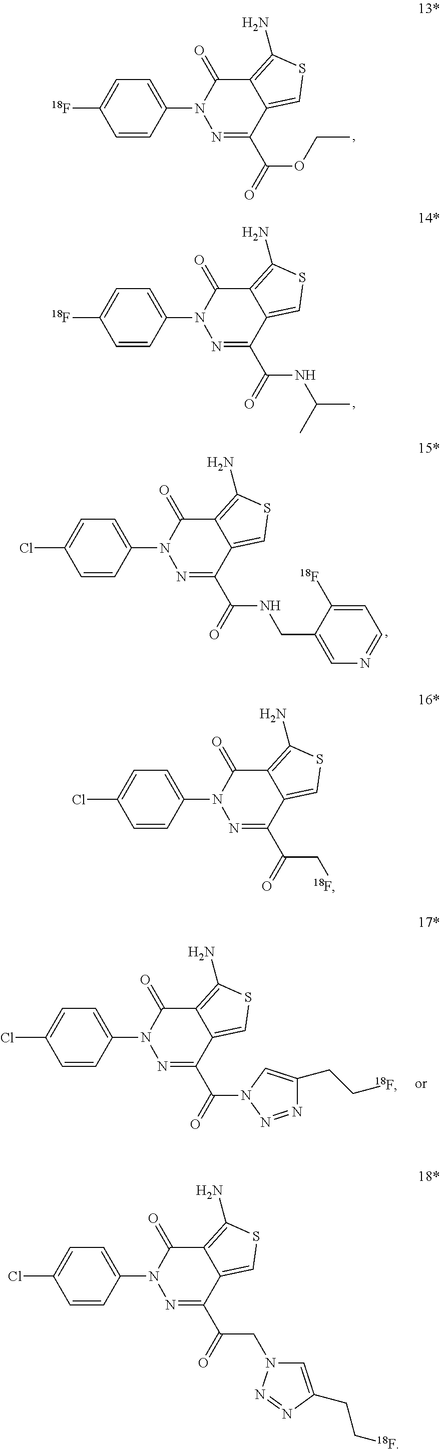

In one or more embodiments of the invention, the compound of Formula (I) is:

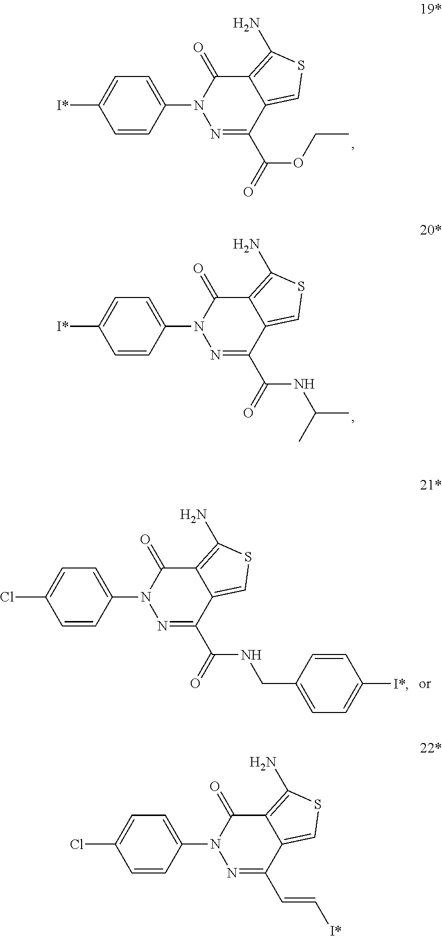

wherein I* is ¹²³I, ¹²⁴I, or ¹²⁵I; more preferably, ¹²³I or ¹²⁵I; more preferably, ¹²³I.

In one or more embodiments of the invention, the compound of Formula (I) is:

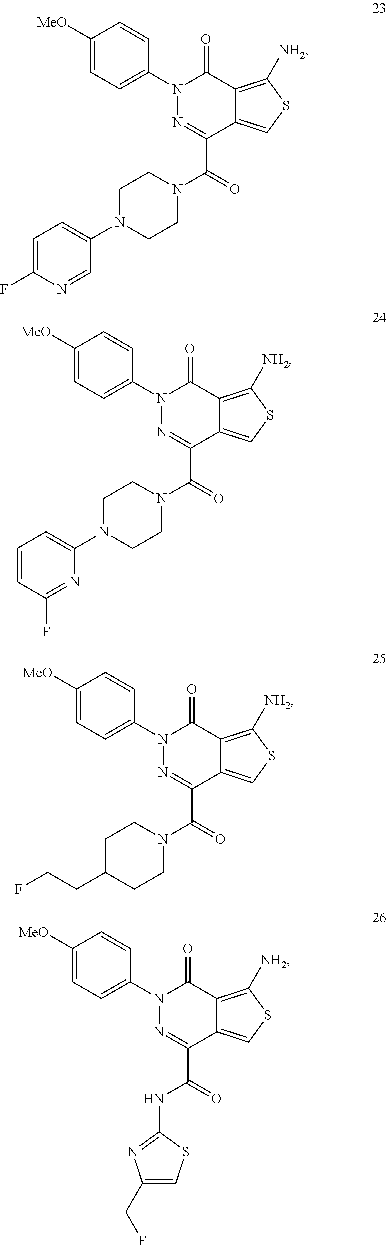

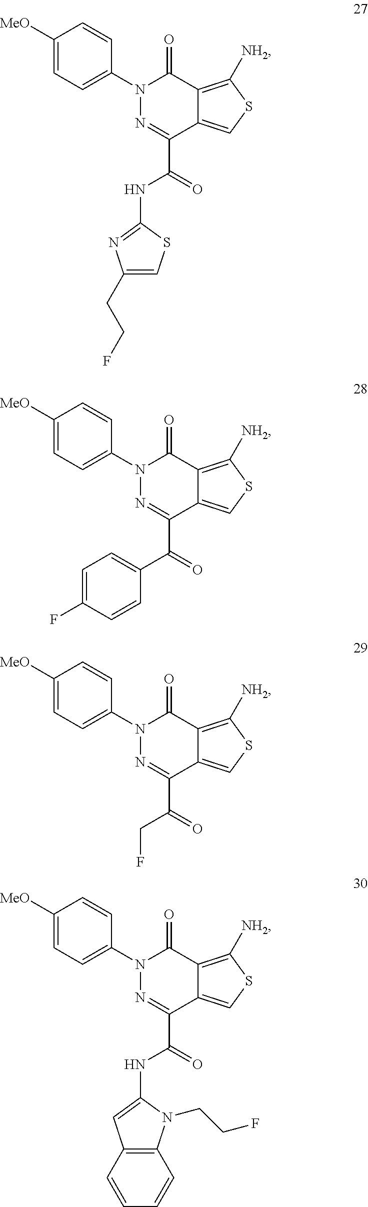 [SMILES: COc1ccc(-n2nc(C(=O)Nc3nc(CCF)cs3)c3csc(N)c3c2=O)cc1 ; COc1ccc(-n2nc(C(=O)c3ccc(F)cc3)c3csc(N)c3c2=O)cc1 ; COc1ccc(-n2nc(C(=O)CF)c3csc(N)c3c2=O)cc1 ; COc1ccc(-n2nc(C(=O)Nc3cc4ccccc4n3CCF)c3csc(N)c3c2=O)cc1]

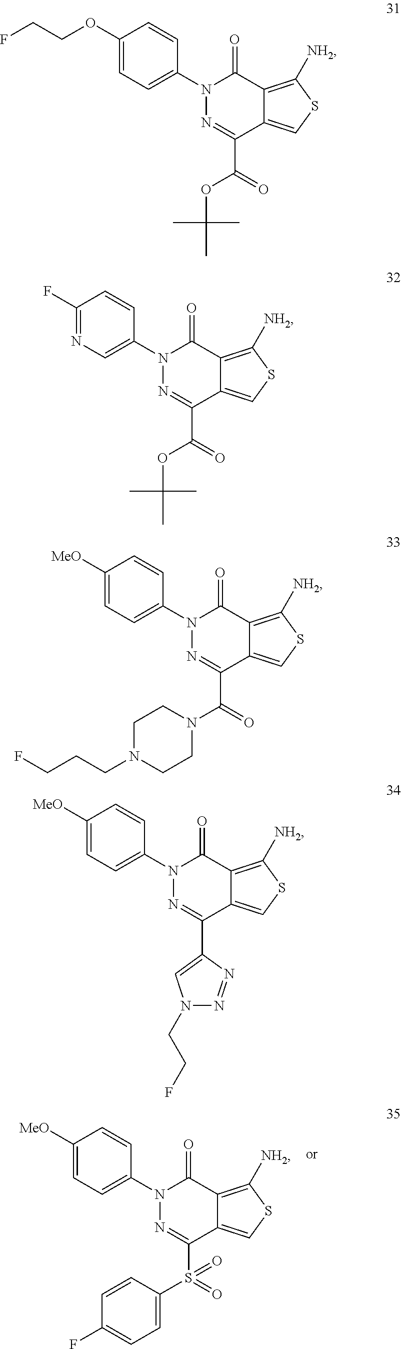

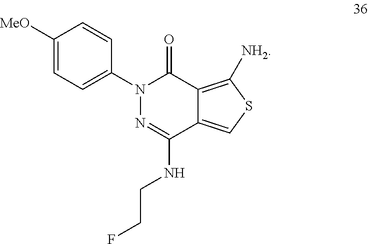 [SMILES: COc1ccc(-n2nc(NCCF)c3csc(N)c3c2=O)cc1]

In one or more embodiments of the invention, the compound of Formula (I) is:

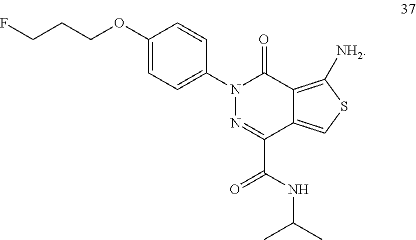 [SMILES: CC(C)NC(=O)c1nn(-c2ccc(OCCCF)cc2)c(=O)c2c(N)scc12]

In one or more embodiments of the invention, the compound of Formula (I) is:

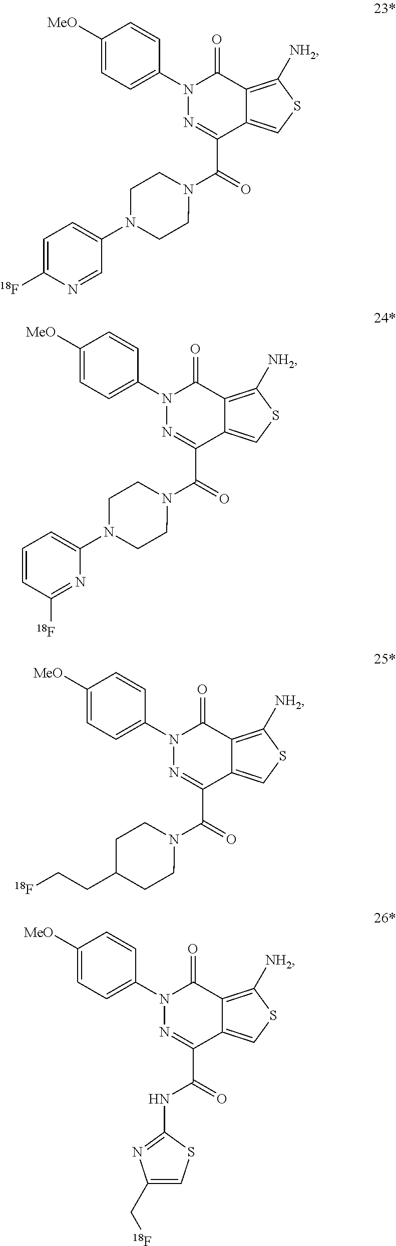

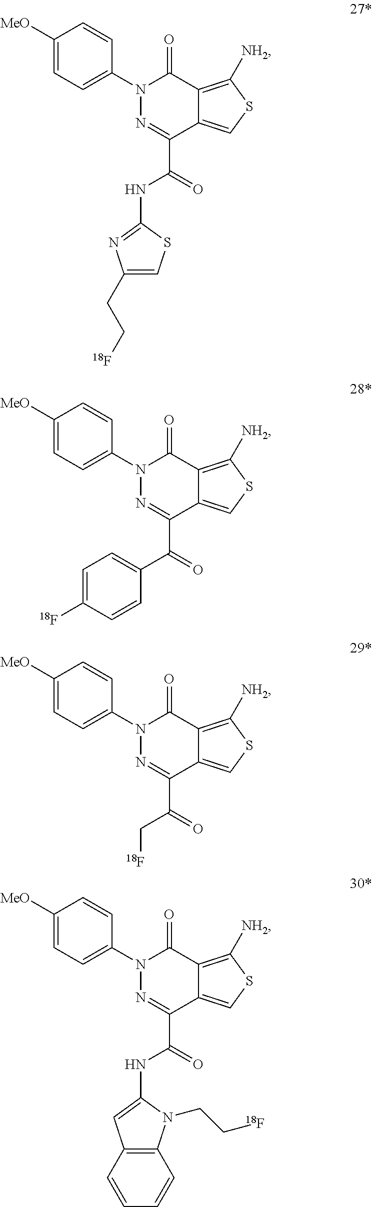 [SMILES: COc1ccc(-n2nc(C(=O)Nc3nc(CC[18F])cs3)c3csc(N)c3c2=O)cc1 ; COc1ccc(-n2nc(C(=O)c3ccc([18F])cc3)c3csc(N)c3c2=O)cc1 ; COc1ccc(-n2nc(C(=O)C[18F])c3csc(N)c3c2=O)cc1 ; COc1ccc(-n2nc(C(=O)Nc3cc4ccccc4n3CC[18F])c3csc(N)c3c2=O)cc1]

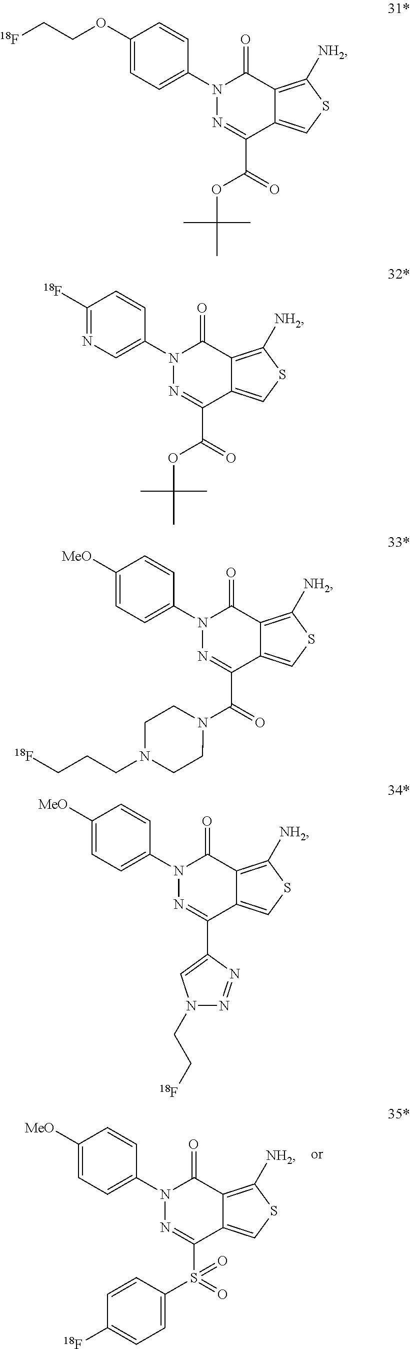

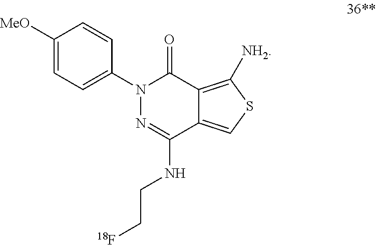 [SMILES: COc1ccc(-n2nc(NCC[18F])c3csc(N)c3c2=O)cc1]

In one or more embodiments of the invention, the compound of Formula (I) is:

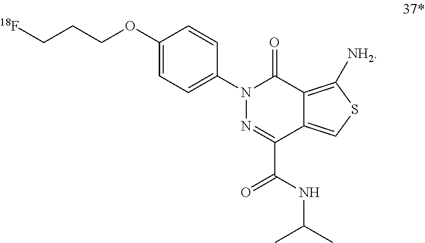 [SMILES: CC(C)NC(=O)c1nn(-c2ccc(OCCC[18F])cc2)c(=O)c2c(N)scc12]

In one or more embodiments of the invention, the compound of Formula (I) is:

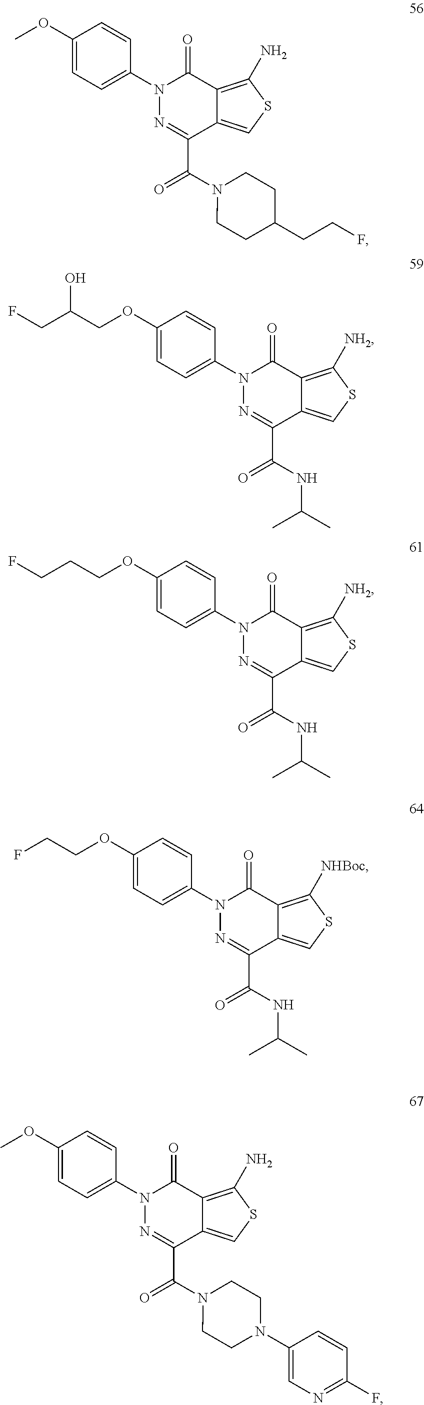 [SMILES: COc1ccc(-n2nc(C(=O)N3CCC(CCF)CC3)c3csc(N)c3c2=O)cc1 ; CC(C)NC(=O)c1nn(-c2ccc(OCC(O)CF)cc2)c(=O)c2c(N)scc12 ; CC(C)NC(=O)c1nn(-c2ccc(OCCCF)cc2)c(=O)c2c(N)scc12 ; CC(C)NC(=O)c1nn(-c2ccc(OCCF)cc2)c(=O)c2c(NC(=O)OC(C)(C)C)scc12 ; COc1ccc(-n2nc(C(=O)N3CCN(c4ccc(F)nc4)CC3)c3csc(N)c3c2=O)cc1]

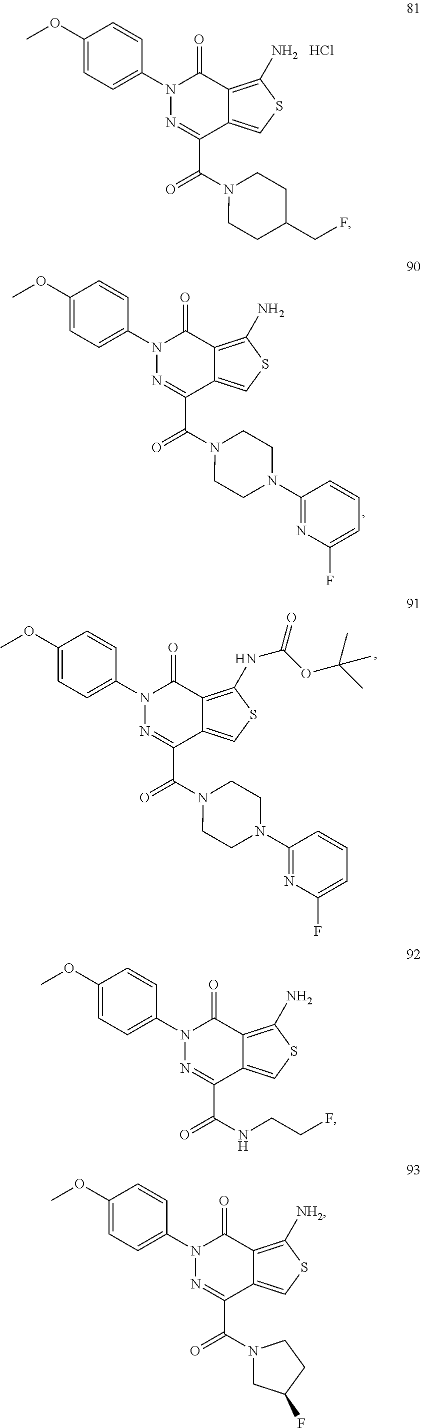

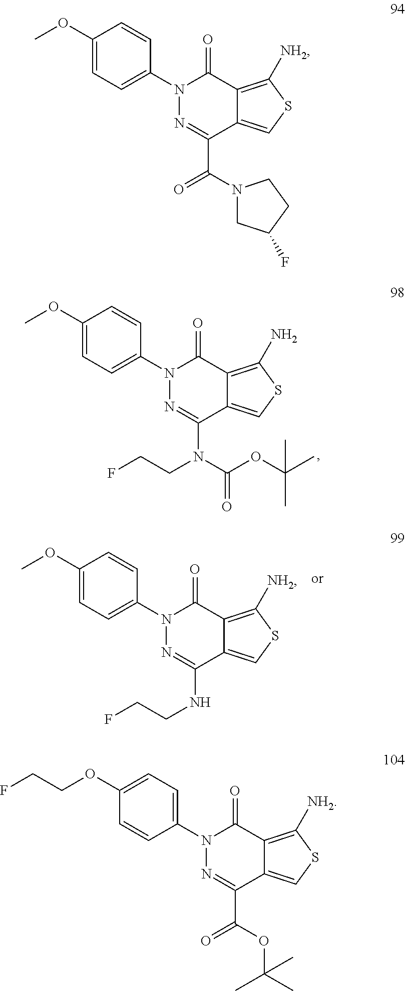

In one or more embodiments of the invention, the compound of Formula (I) is:

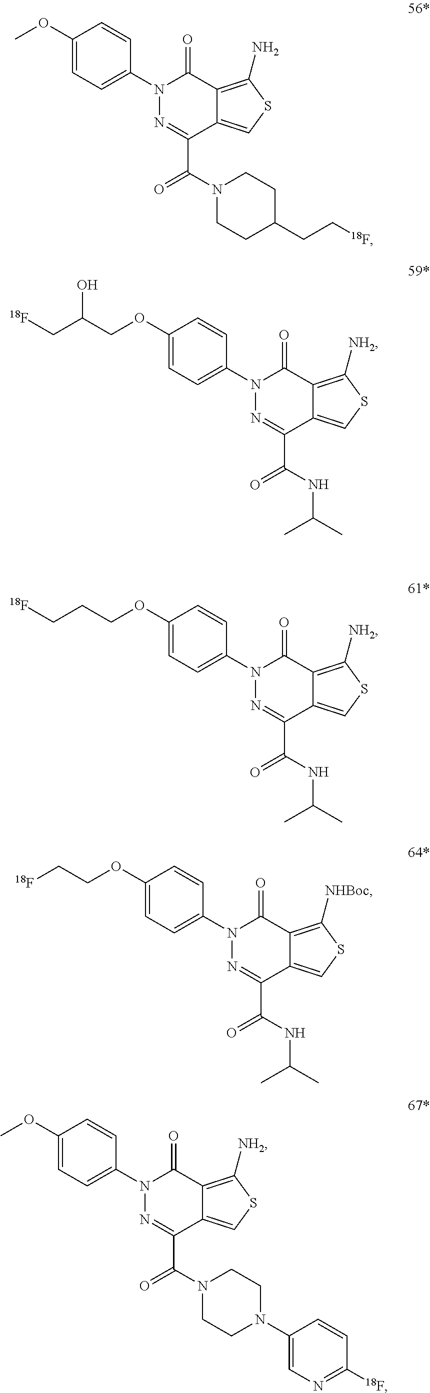

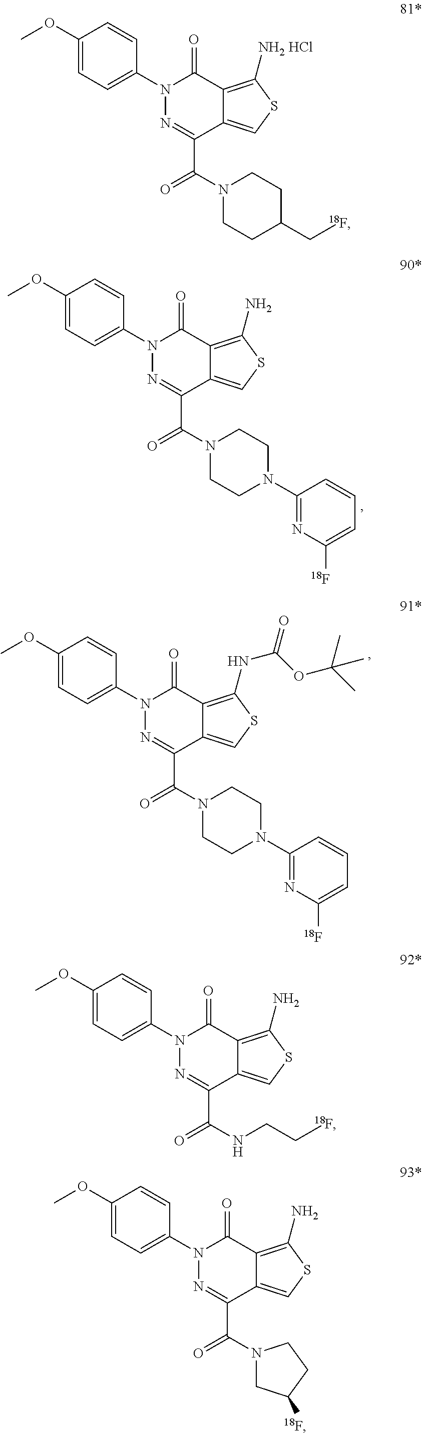

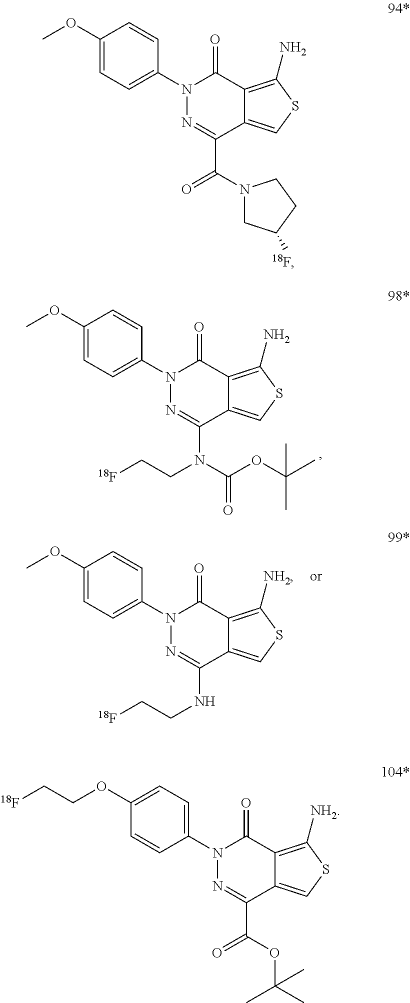

According to the present invention, for a compound of the invention described herein, a halogen is selected from F, Cl, Br, and I; preferably, F.

The invention provides a radiolabelled derivative of a compound of the invention as described herein. According to the present invention, a “radiolabelled derivative” of a compound of the invention or a “radiolabelled derivative thereof” is a compound of the invention, as described herein, that comprises a radionuclide (i.e., a compound of the invention that is radiolabelled with a radionuclide. By way of example, a radiolabelled derivative of a compound of Formula (I) is a compound of Formula (I) as described herein wherein at least one of R¹, R², R³, R⁴ and Ar comprises a radionuclide. The radionuclide shall mean any radioisotope known in the art. Preferably the radionuclide is a radioisotope suitable for imaging (e.g., PET, SPECT).

In one embodiment, the radionuclide is a radioisotope suitable for PET imaging. Even more preferably, the radionuclide is ¹¹C, ¹³N, ¹⁵O, ⁶⁸Ga, ⁶²Cu, ¹⁸F, ⁷⁶Br, ¹²⁴I, or ¹²⁵I; even more preferably, the radionuclide is ¹⁸F.

In one embodiment, the radionuclide is a radioisotope suitable for SPECT imaging. Even more preferably, the radionuclide is ^{99m}Tc, ¹¹¹In, ⁶⁷Ga, ²⁰¹Tl, ¹²³I, or ¹³³Xe; even more preferably, the radionuclide is ^{99m}Tc or ¹²³I.

### Intermediates:

The present invention provides pre-cursor or intermediate compounds of Formula II:

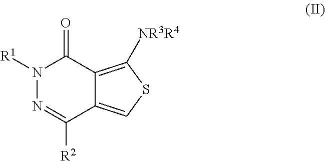

wherein:
R¹ is alkyl or Ar, optionally substituted with at least one alkyl, halogen, hydroxyl, alkoxy, haloalkoxy, acid, ester, amino, nitro, amide, alkoxyhalo or alkyoxyOPg;
R² is independently alkyl, alkynyl, ester, amino, amide, acid, aryl, heteroaryl, aminoalkyl, —C(═O)alkyl, —C(═O)aryl, —C(═O)heteroaryl, —C(═O)heterocycloalkyl, —C(═O)heterocycloalkylAr, —C(═O)(CH₂)ₚOPg, —C(═O)(CH₂)ₚhalo, —C(═O)(CH₂)ₚheterocyclyl, or —SO₂Ar, optionally substituted with an alkyl, alkylhalo, alkylOPg, halogen, nitro, aryl, heteroaryl, heteroaryl(CH₂)ₚhalo, or heteroaryl(CH₂)ₚOPg;
R³ and R⁴ are independently hydrogen, alkyl, alkenyl, alkynyl, aryl, or heteroaryl;
Ar is an aryl, heteroaryl, cycloalkyl, or heterocycloalkyl group;
p is an integer from 0-10; preferably, 0-5; more preferably, 0-3;
Pg is H, a protecting or leaving group.
The protecting or leaving group may be any protecting or leaving group known in the art. Examples of suitable protecting or leaving groups include, but are not limited to, tosylate (OTs), BOC, Fmoc, Cbz, acetyl (Ac) and paramthoxybenzyl (PMB).

Examples of a pre-cursor or intermediate compounds of the invention include:

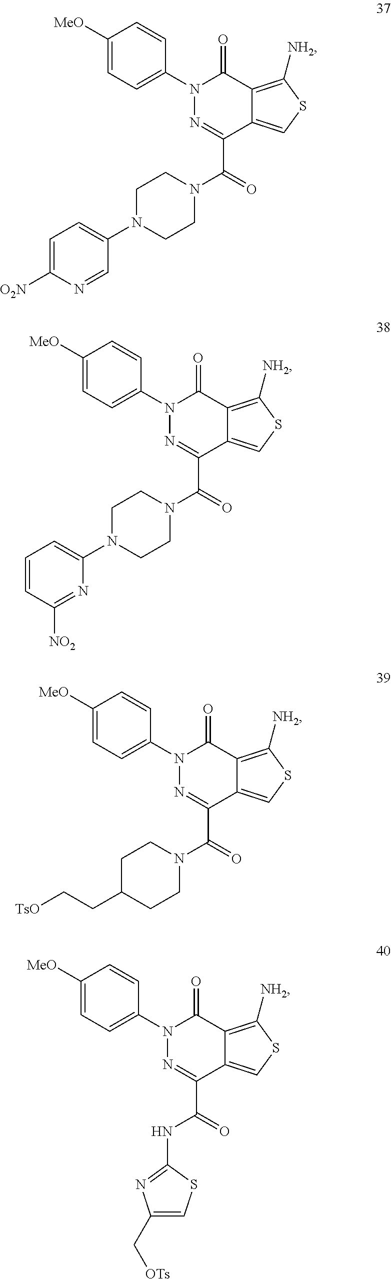 [SMILES: COc1ccc(-n2nc(C(=O)N3CCN(c4ccc([N+](=O)[O-])nc4)CC3)c3csc(N)c3c2=O)cc1 ; COc1ccc(-n2nc(C(=O)N3CCN(c4cccc([N+](=O)[O-])n4)CC3)c3csc(N)c3c2=O)cc1 ; COc1ccc(-n2nc(C(=O)N3CCC(CCOS(=O)(=O)c4ccc(C)cc4)CC3)c3csc(N)c3c2=O)cc1 ; COc1ccc(-n2nc(C(=O)Nc3nc(COS(=O)(=O)c4ccc(C)cc4)cs3)c3csc(N)c3c2=O)cc1]

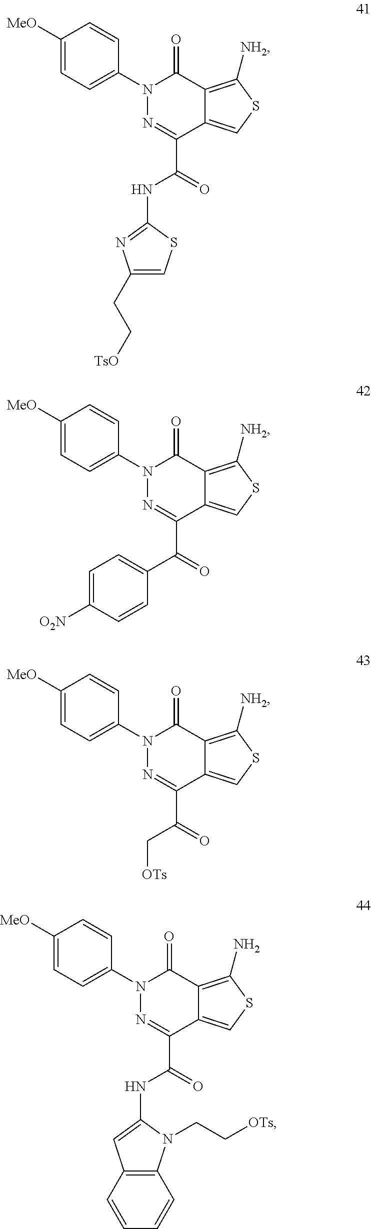 [SMILES: COc1ccc(-n2nc(C(=O)Nc3nc(CCOS(=O)(=O)c4ccc(C)cc4)cs3)c3csc(N)c3c2=O)cc1 ; COc1ccc(-n2nc(C(=O)c3ccc([N+](=O)[O-])cc3)c3csc(N)c3c2=O)cc1 ; COc1ccc(-n2nc(C(=O)COS(=O)(=O)c3ccc(C)cc3)c3csc(N)c3c2=O)cc1 ; COc1ccc(-n2nc(C(=O)Nc3cc4ccccc4n3CCOS(=O)(=O)c3ccc(C)cc3)c3csc(N)c3c2=O)cc1]

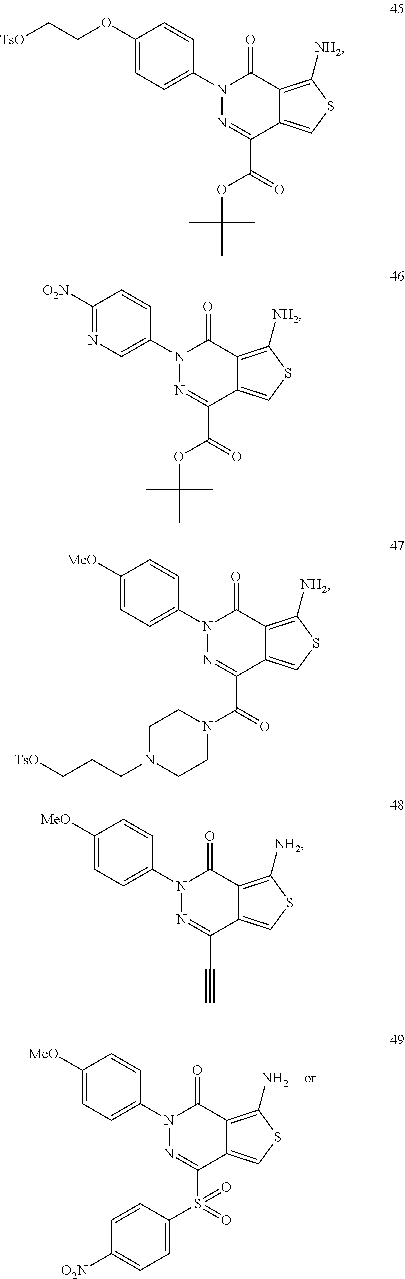

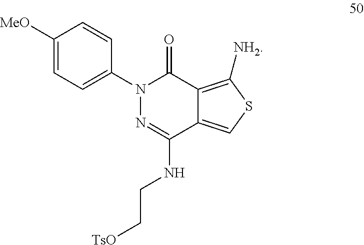 [SMILES: COc1ccc(-n2nc(NCCOS(=O)(=O)c3ccc(C)cc3)c3csc(N)c3c2=O)cc1]

The present invention further provides a pre-cursor or intermediate compound of the formula:

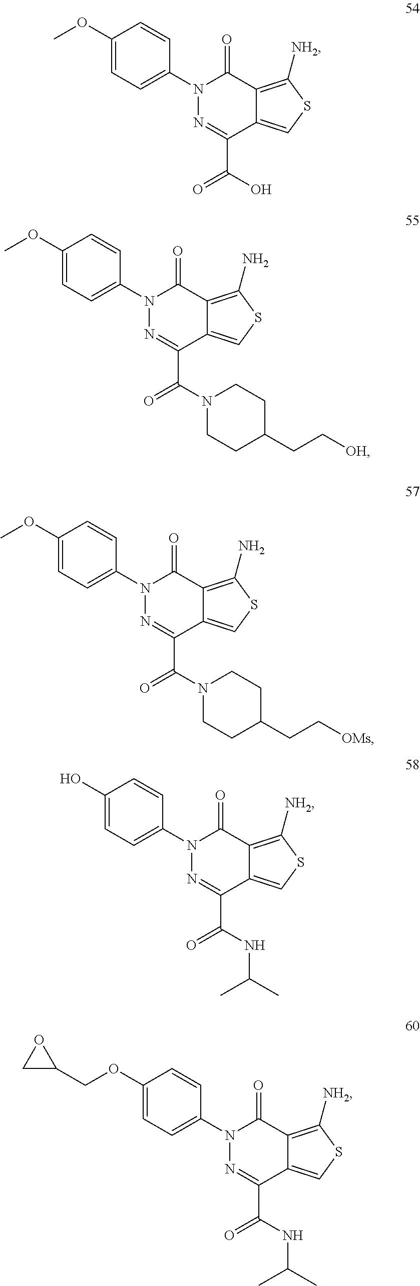 [SMILES: COc1ccc(-n2nc(C(=O)O)c3csc(N)c3c2=O)cc1 ; COc1ccc(-n2nc(C(=O)N3CCC(CCO)CC3)c3csc(N)c3c2=O)cc1 ; COc1ccc(-n2nc(C(=O)N3CCC(CCOS(C)(=O)=O)CC3)c3csc(N)c3c2=O)cc1 ; CC(C)NC(=O)c1nn(-c2ccc(O)cc2)c(=O)c2c(N)scc12 ; CC(C)NC(=O)c1nn(-c2ccc(OCC3CO3)cc2)c(=O)c2c(N)scc12]

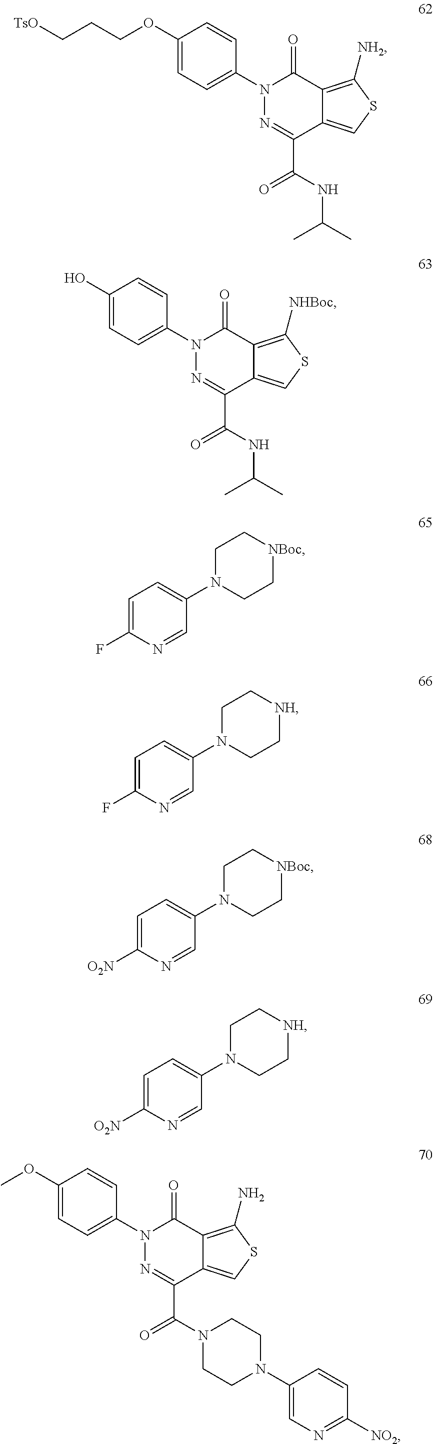

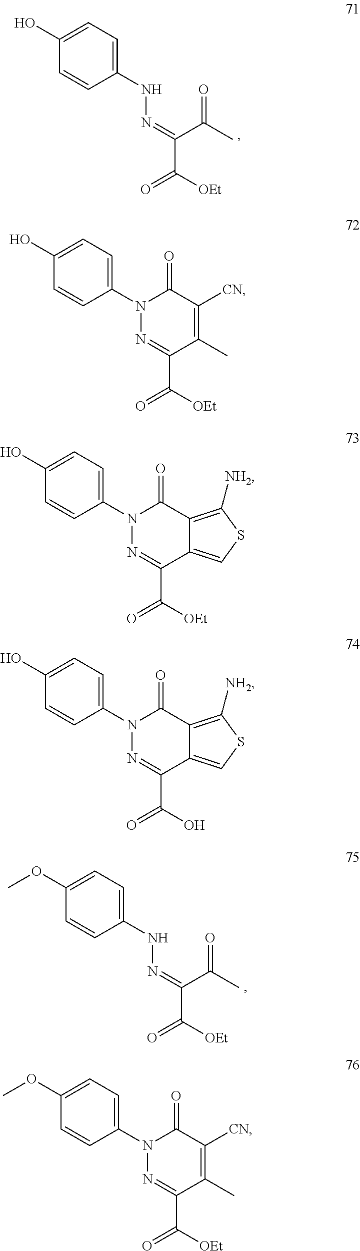

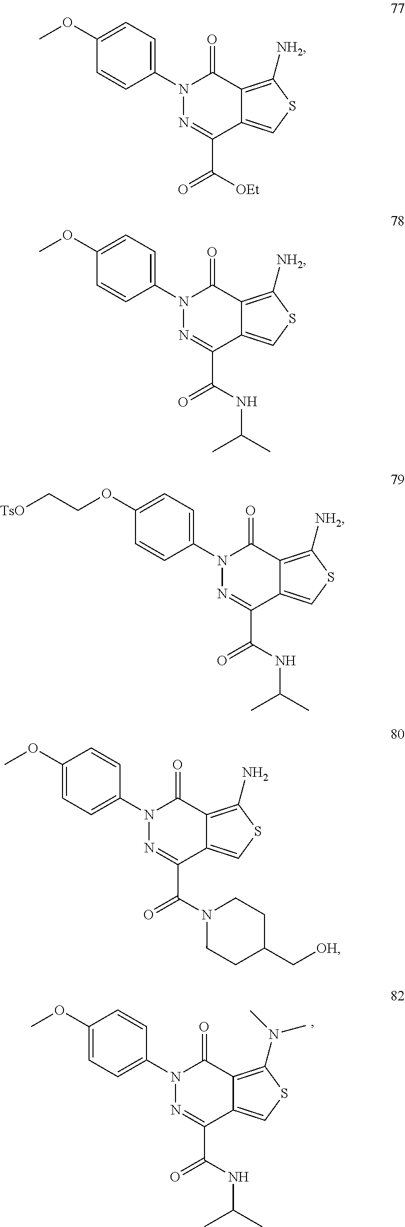

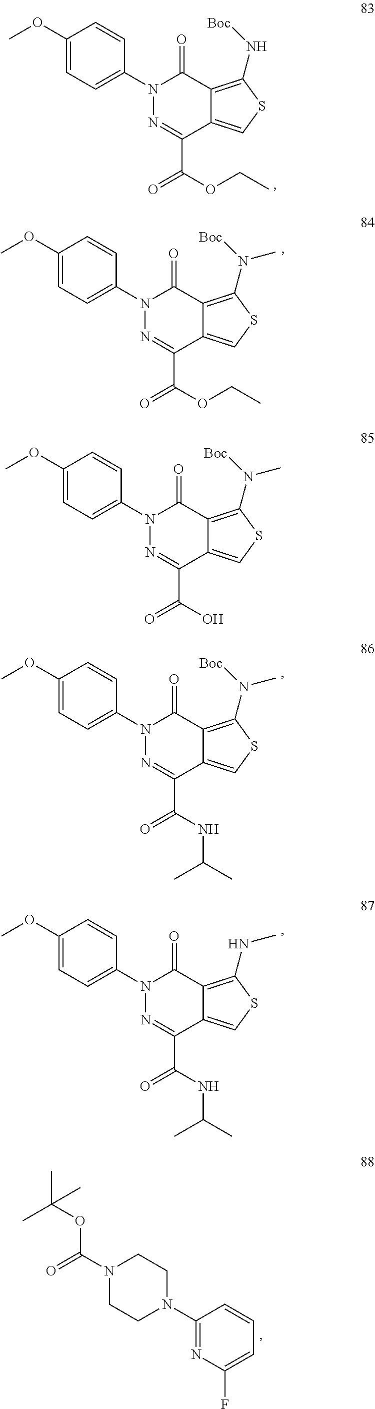

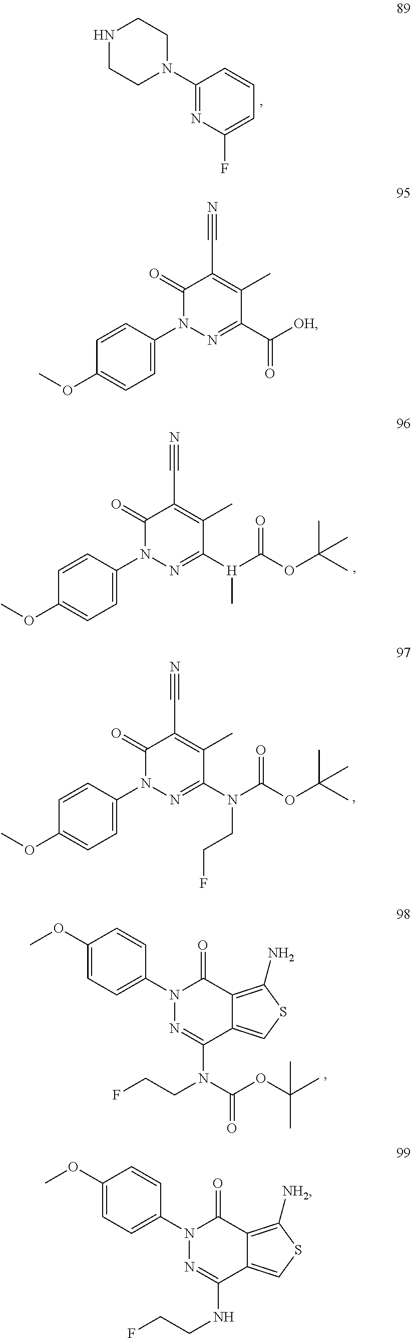

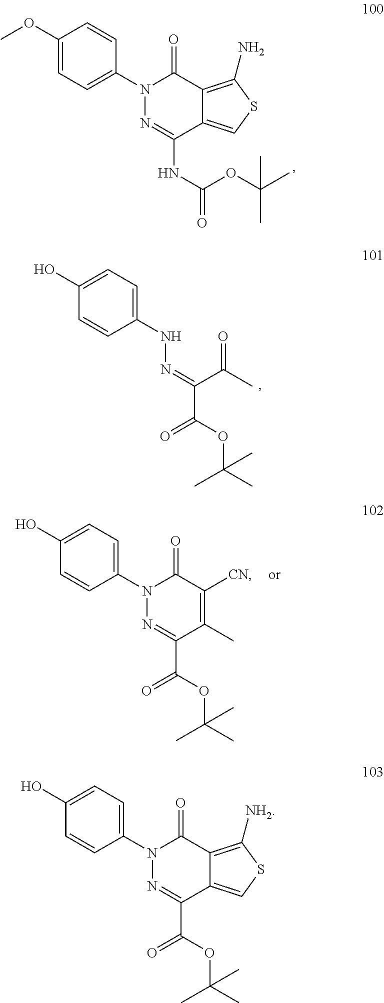 [SMILES: COc1ccc(-n2nc(NC(=O)OC(C)(C)C)c3csc(N)c3c2=O)cc1 ; CC(=O)/C(=N\Nc1ccc(O)cc1)C(=O)OC(C)(C)C ; Cc1c(C(=O)OC(C)(C)C)nn(-c2ccc(O)cc2)c(=O)c1C#N ; CC(C)(C)OC(=O)c1nn(-c2ccc(O)cc2)c(=O)c2c(N)scc12]

### Pharmaceutical or Radiopharmaceutical Composition

The present invention provides a pharmaceutical or radiopharmaceutical composition comprising a compound of the invention as described herein together with a pharmaceutically acceptable carrier, excipient, or biocompatible carrier. According to the invention when a compound of the invention is a radiolabelled derivative, the pharmaceutical composition is a radiopharmaceutical composition.

The present invention further provides a pharmaceutical or radiopharmaceutical composition comprising a compound of the invention as described herein together with a pharmaceutically acceptable carrier, excipient, or biocompatible carrier suitable for mammalian administration.

As would be understood by one of skill in the art, the pharmaceutically acceptable carrier or excipient can be any pharmaceutically acceptable carrier or excipient known in the art.

The “biocompatible carrier” can be any fluid, especially a liquid, in which a compound of the invention can be suspended or dissolved, such that the pharmaceutical composition is physiologically tolerable, e.g., can be administered to the mammalian body without toxicity or undue discomfort. The biocompatible carrier is suitably an injectable carrier liquid such as sterile, pyrogen-free water for injection; an aqueous solution such as saline (which may advantageously be balanced so that the final product for injection is either isotonic or not hypotonic); an aqueous solution of one or more tonicity-adjusting substances (e.g., salts of plasma cations with biocompatible counterions), sugars (e.g., glucose or sucrose), sugar alcohols (e.g., sorbitol or mannitol), glycols (e.g., glycerol), or other non-ionic polyol materials (e.g., polyethyleneglycols, propylene glycols and the like). The biocompatible carrier may also comprise biocompatible organic solvents such as ethanol. Such organic solvents are useful to solubilise more lipophilic compounds or formulations. Preferably the biocompatible carrier is pyrogen-free water for injection, isotonic saline or an aqueous ethanol solution. The pH of the biocompatible carrier for intravenous injection is suitably in the range 4.0 to 10.5.

The pharmaceutical or radiopharmaceutical composition may be administered parenterally, i.e., by injection, and is most preferably an aqueous solution. Such a composition may optionally contain further ingredients such as buffers; pharmaceutically acceptable solubilisers (e.g., cyclodextrins or surfactants such as Pluronic, Tween or phospholipids); pharmaceutically acceptable stabilisers or antioxidants (such as ascorbic acid, gentisic acid or para-aminobenzoic acid). Where a compound of the invention is provided as a radiopharmaceutical composition, the method for preparation of said compound may further comprise the steps required to obtain a radiopharmaceutical composition, e.g., removal of organic solvent, addition of a biocompatible buffer and any optional further ingredients. For parenteral administration, steps to ensure that the radiopharmaceutical composition is sterile and apyrogenic also need to be taken. Such steps are well-known to those of skill in the art.

### Preparation of a Compound of the Invention

A compound of the invention may be prepared by any means known in the art including, but not limited to, nucleophilic aromatic substitution, nucleophilic aliphatic substitution, and click chemistry.

In one embodiment of the invention, a compound of the invention may be halogenated or radiolabeled with a radionuclide by nucleophilic aromatic substitution or nucleophilic aliphatic substitution of an appropriate leaving group with the desired halogen or radionuclide. Examples of suitable leaving groups for nucleophilic aromatic substitution include, but are not limited to, Cl, Br, F, NO₂, ArI⁺ and ⁺N(R)₄. Examples of suitable leaving groups for nucleophilic aliphatic substitution include, but are not limited to, I, Br, Cl, OTs (tosylate), OTf (triflate), BsO(brosylate), OMs(Mesylate), and NsO (nosylate).

In one embodiment of the invention, a compound of the invention may be directly labelled with ¹⁸F via activated aromatic rings. This approach would require a protection of the essential amino group during radiolabelling.

In one embodiment, a compound of the invention may be prepared according to the following Scheme I:

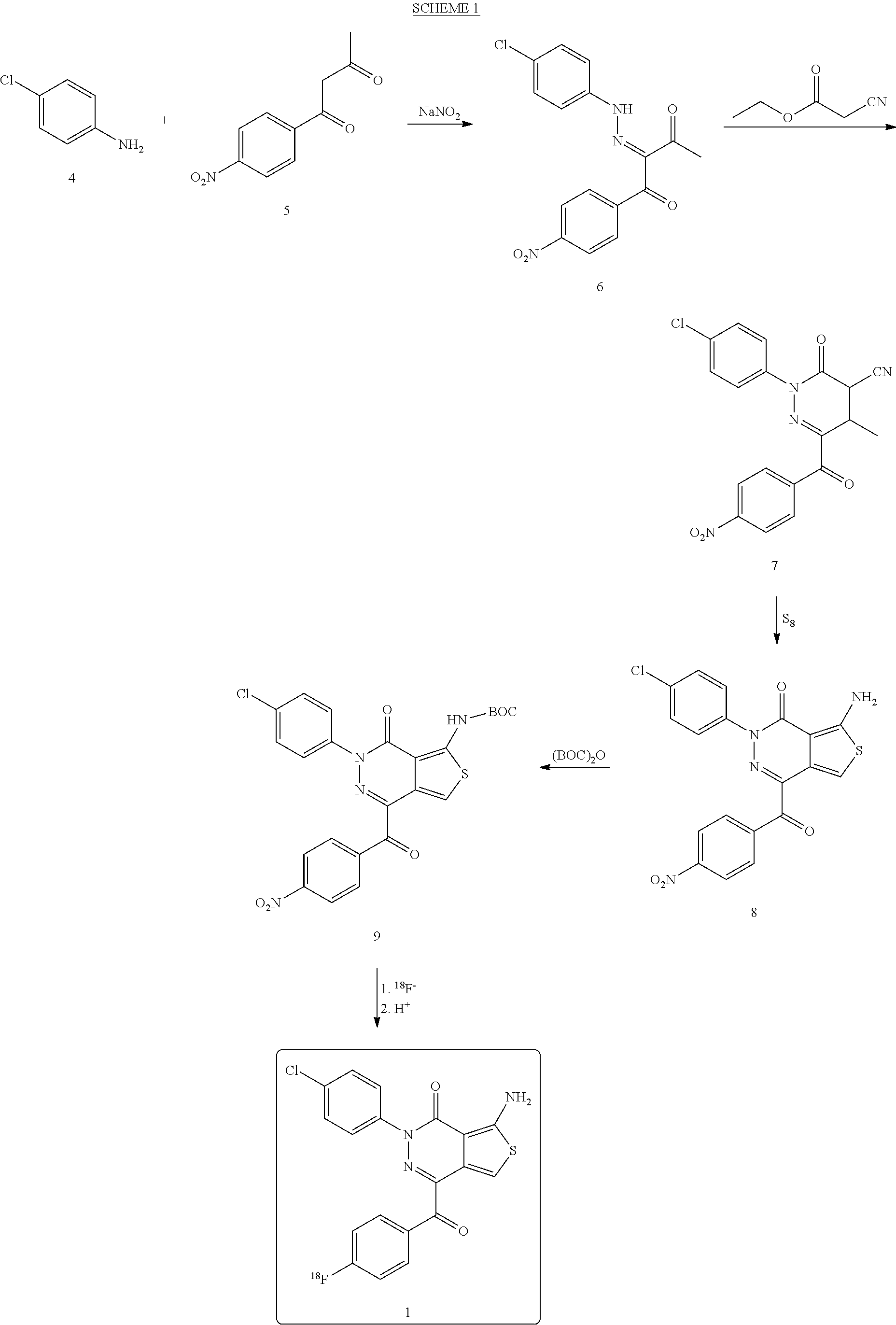

In one embodiment, a compound of the invention may be prepared according to the following Scheme II:

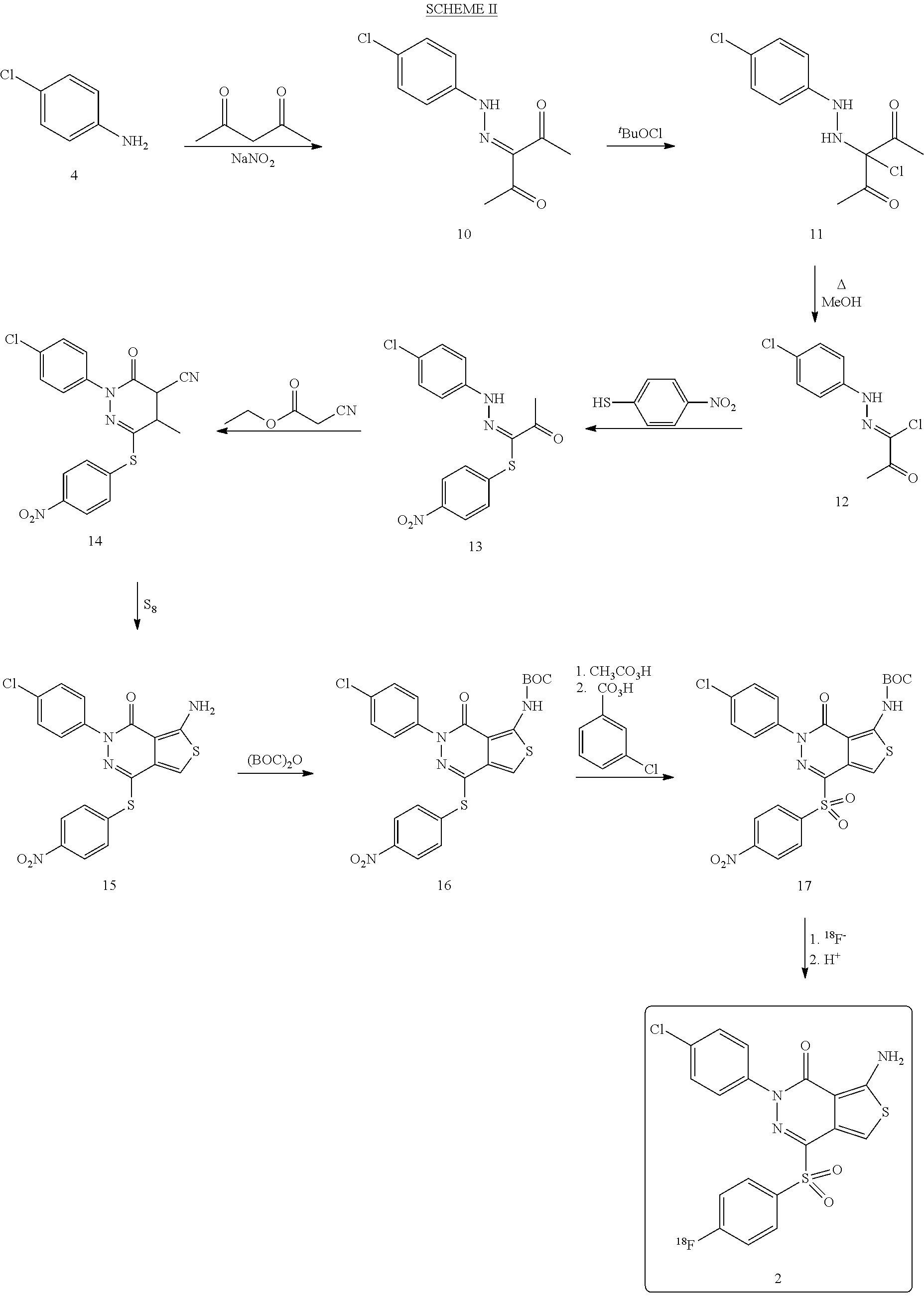

In one embodiment, a compound of the invention may be prepared according to the following Scheme III:

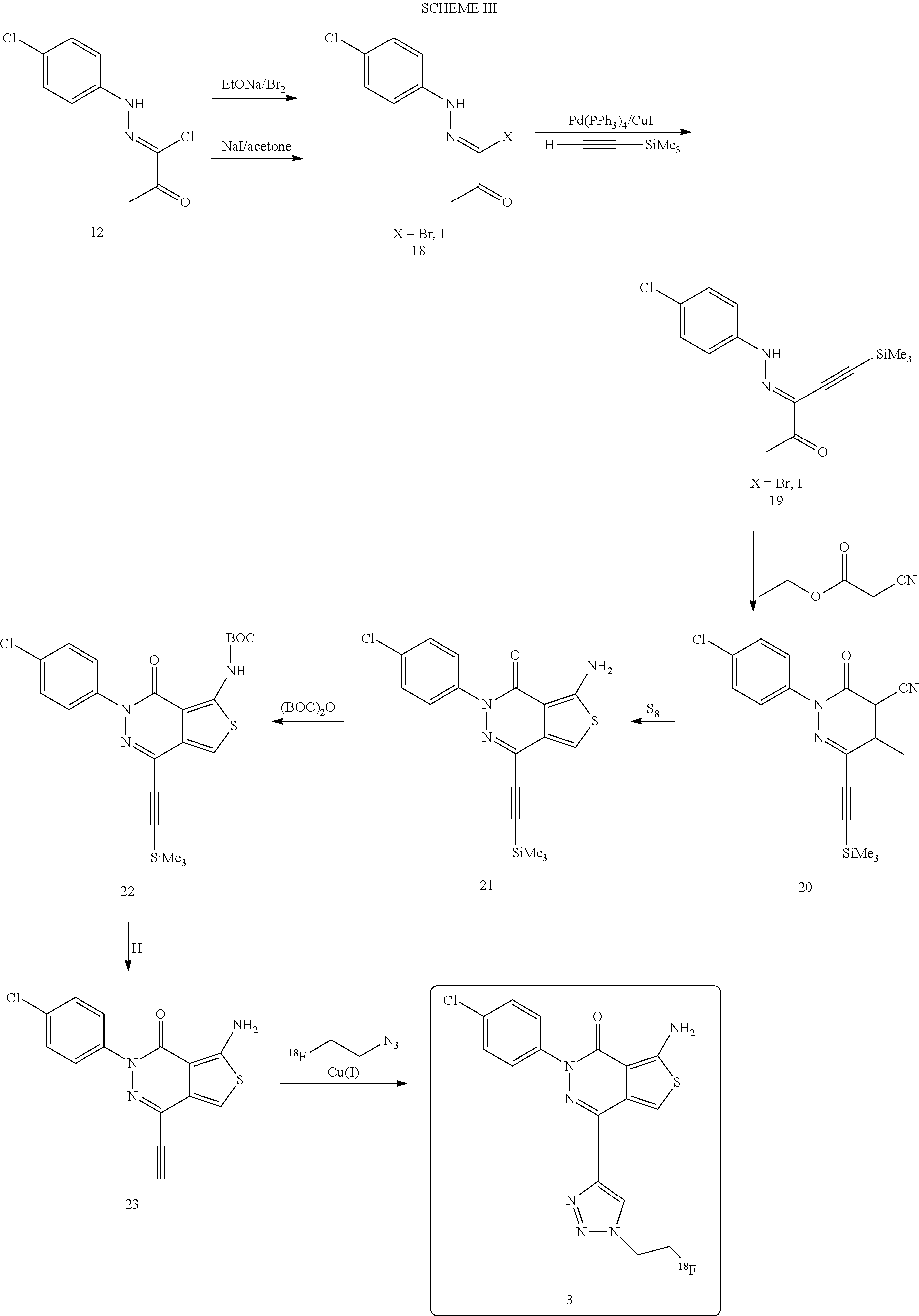

In one embodiment, a compound of the invention may be prepared according to the following Scheme IV:

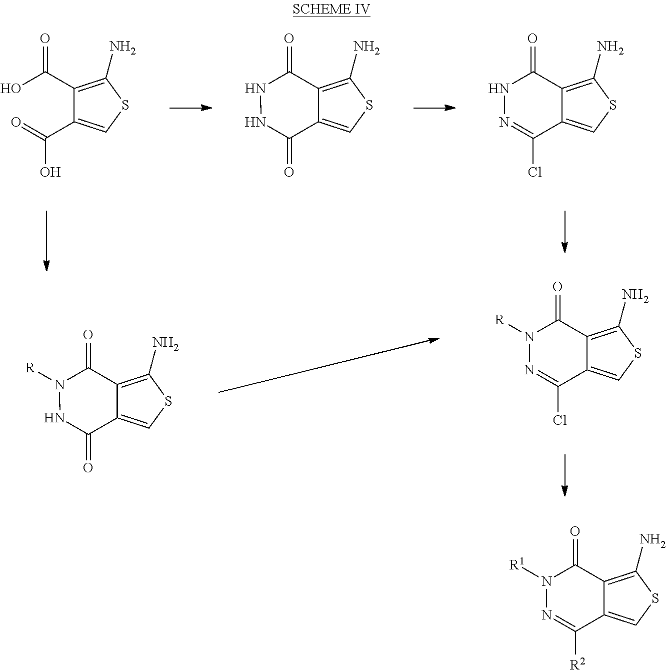

By way of example, the radioisotope [¹⁸F]-fluoride ion (¹⁸F⁻) is normally obtained as an aqueous solution from the nuclear reaction ¹⁸O(p,n)¹⁸F and is made reactive by the addition of a cationic counterion and the subsequent removal of water. Suitable cationic counterions should possess sufficient solubility within the anhydrous reaction solvent to maintain the solubility of 18F⁻. Therefore, counterions that have been used include large but soft metal ions such as rubidium or caesium, potassium complexed with a cryptand such as Kryptofix™, or tetraalkylammonium salts. A preferred counterion is potassium complexed with a cryptand such as Kryptofix™ because of its good solubility in anhydrous solvents and enhanced ¹⁸F⁻reactivity. ¹⁸F can also be introduced by nucleophilic displacement of a suitable leaving group such as a halogen or tosylate group. A more detailed discussion of well-known ¹⁸F labelling techniques can be found in Chapter 6 of the “Handbook of Radiopharmaceuticals” (2003; John Wiley and Sons: M. J. Welch and C. S. Redvanly, Eds.). Similar methods may be used to radiolabel a compound of the invention with other radioisotopes including the PET and SPECT radioisotopes described herein.

### Automated Synthesis

In one embodiment, the method to prepare a radiolabelled derivative of the invention, each as described herein, is automated. For example, [¹⁸F]-labeled compounds of the invention may be conveniently prepared in an automated fashion by means of an automated radiosynthesis apparatus. There are several commercially-available examples of such platform apparatus, including TRACERlab™ (e.g., TRACERlab™ MX) and FASTlab™ (both from GE Healthcare Ltd.). Such apparatus commonly comprises a “cassette”, often disposable, in which the radiochemistry is performed, which is fitted to the apparatus in order to perform a radiosynthesis. The cassette normally includes fluid pathways, a reaction vessel, and ports for receiving reagent vials as well as any solid-phase extraction cartridges used in post-radiosynthetic clean up steps. Optionally, in a further embodiment of the invention, the automated radiosynthesis apparatus can be linked to a high performance liquid chromatograph (HPLC). The present invention therefore provides a cassette for the automated synthesis of a compound of the invention.

### Imaging Method

The radiolabelled derivative of the invention, as described herein, may bind to NFTs or tau aggregates and aid in identifying the amount of NFTs/tau aggregates present which in turn may correlate with the stage of AD.

The present invention thus provides a method of imaging comprising the step of administering a radiolabelled derivative of the invention, as described herein, to a subject and detecting said radiolabelled derivative of the invention in said subject. The present invention further provides a method of detecting tau aggregates in vitro or in vivo using a radiolabelled derivative of the invention, as described herein. Hence the present invention provides better tools for early detection and diagnosis of Alzheimers disease. The present invention also provides better tools for monitoring the progression of Alzheimers disease and the effect of treatment.

As would be understood by one of skill in the art the type of imaging (e.g., PET, SPECT) will be determined by the nature of the radioisotope. For example, if the radiolabelled derivative of the invention contains ¹⁸F it will be suitable for PET imaging.

Thus the invention provides a method of detecting tau aggregates in vitro or in vivo comprising the steps of:
i) administering to a subject a radiolabelled derivative of the invention as defined herein;
ii) allowing said a radiolabelled derivative of the invention to bind to NFTs in said subject;
iii) detecting signals emitted by said radioisotope in said bound radiolabelled derivative of the invention;
iv) generating an image representative of the location and/or amount of said signals; and,
v) determining the distribution and extent of said tau aggregates in said subject.

The step of “administering” a radiolabelled derivative of the invention is preferably carried out parenterally, and most preferably intravenously. The intravenous route represents the most efficient way to deliver the compound throughout the body of the subject. Intravenous administration neither represents a substantial physical intervention nor a substantial health risk to the subject. The radiolabelled derivative of the invention is preferably administered as the radiopharmaceutical composition of the invention, as defined herein. The administration step is not required for a complete definition of the imaging method of the invention. As such, the imaging method of the invention can also be understood as comprising the above-defined steps (ii)-(v) carried out on a subject to whom a radiolabelled derivative of the invention has been pre-administered.

Following the administering step and preceding the detecting step, the radiolabelled derivative of the invention is allowed to bind to the tau aggregates. For example, when the subject is an intact mammal, the radiolabelled derivative of the invention will dynamically move through the mammal's body, coming into contact with various tissues therein. Once the radiolabelled derivative of the invention comes into contact with the tau aggregates it will bind to the tau aggregates.

The “detecting” step of the method of the invention involves detection of signals emitted by the radioisotope comprised in the radiolabelled derivative of the invention by means of a detector sensitive to said signals, e.g., a PET camera. This detection step can also be understood as the acquisition of signal data.

The “generating” step of the method of the invention is carried out by a computer which applies a reconstruction algorithm to the acquired signal data to yield a dataset. This dataset is then manipulated to generate images showing the location and/or amount of signals emitted by the radioisotope. The signals emitted directly correlate with the amount of enzyme or neoplastic tissue such that the “determining” step can be made by evaluating the generated image.

The “subject” of the invention can be any human or animal subject. Preferably the subject of the invention is a mammal. Most preferably, said subject is an intact mammalian body in vivo. In an especially preferred embodiment, the subject of the invention is a human.

The “disease state associated with the tau aggregates” can be MCI (mild cognitive impairment), dementia or Alzheimers disease.

### EXAMPLES

Unless set forth otherwise, all materials are commercially available. Abbreviations have the following meanings:
BINAP 1,2-di(naphthalen-2-yl)-1,1,2,2-tetraphenyldiphosphine
BOP (benzotriazol-1-yloxy)tris(dimethylamino) phosphonium hexafluoro phosphate
DCM Dichloromethane
DIPEA N,N-diisopropylethylamine
DMF Dimethylformamide
DMSO DimethylSulfoxide
HPLC High Performance Liquid Chromatography
Kryptofix 4,7,13,16,21,24-Hexaoxa-1,10-diazabicyclo[8.8.8]hexacosane
PBS Phosphate Buffered Saline
QC HPLC Quality Control High-performance Liquid Chromatography
TLC Thin Layer Chromatography
TFA Trifluoroacetic acid

### Example 1

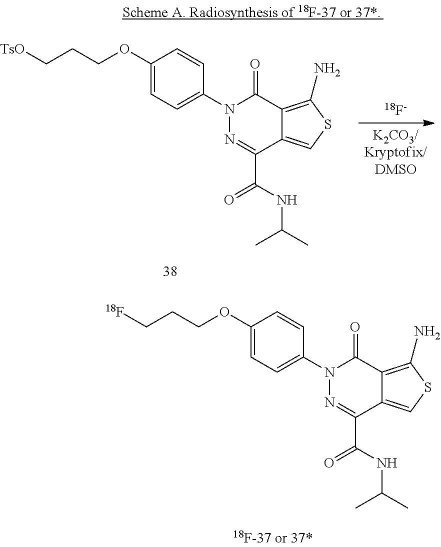

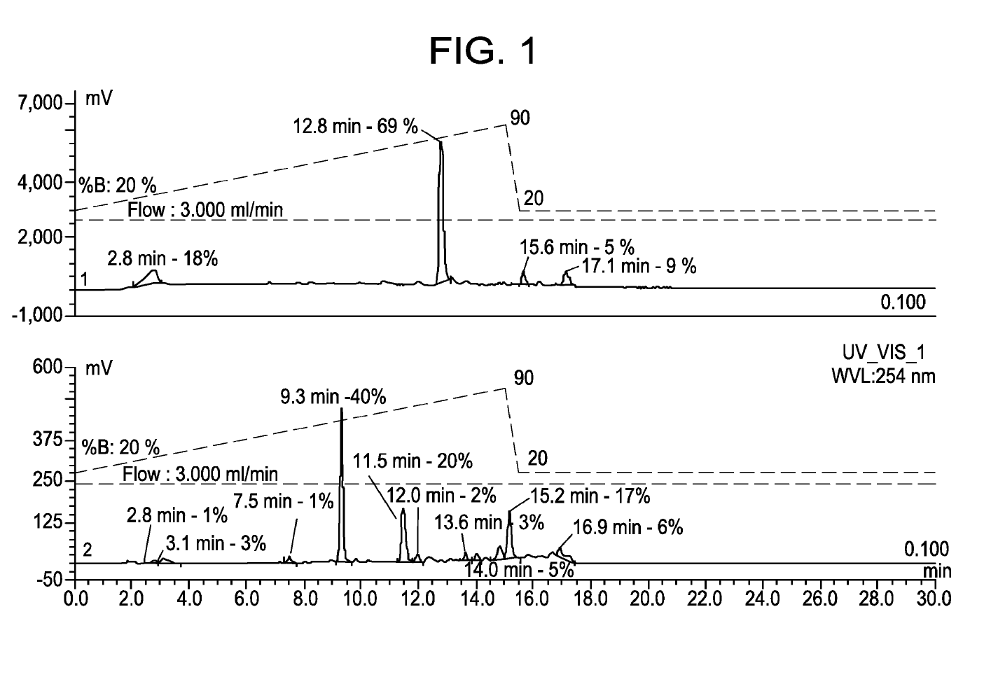

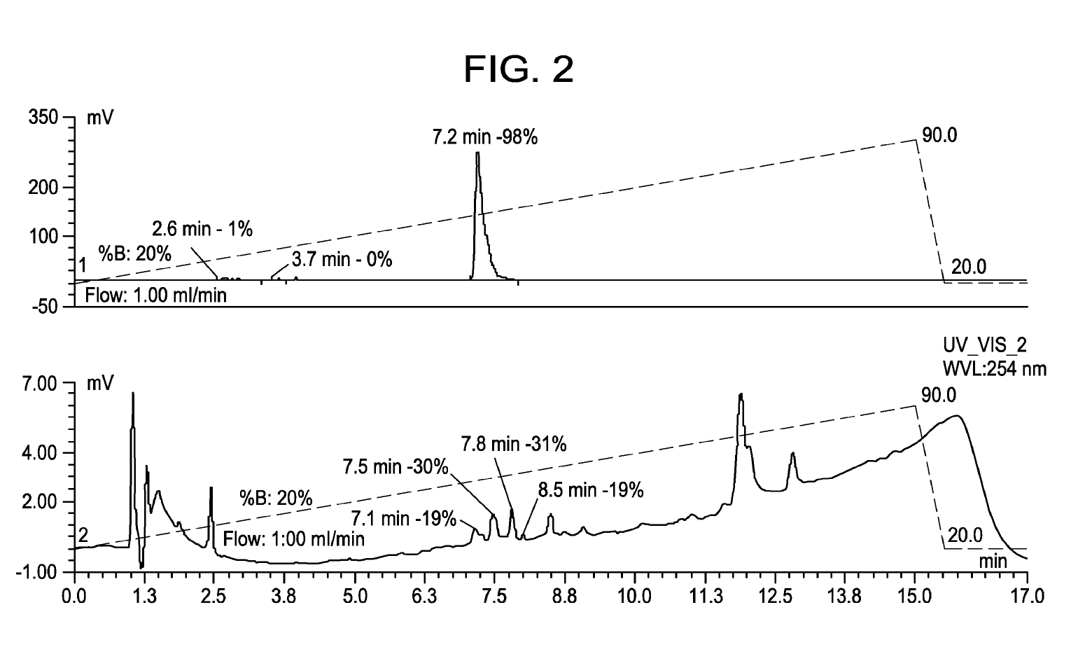

Fluorine-18 is produced in a cyclotron using the ¹⁸O(p,n)¹⁸F nuclear reaction via proton irradiation of a target containing enriched [¹⁸O]H₂O. A Wheaton vial (3 mL) is charged with Kryptofix (5 mg, 13.3 mmol), potassium carbonate (1 mg, 7.2 mmol), acetonitrile (1 mL), and ¹⁸F-containing water (100 μL, 335 MBq). The vial is heated to 100° C. and the solvent removed using a stream of nitrogen (100 mL/min) Acetonitrile (0.5 mL) is added and again evaporated to dryness using a stream of nitrogen. The procedure is repeated two times. The vial is cooled to room temperature and a solution of tosylate 38 (2.0 mg, 3.6 mmol) in anhydrous DMSO (0.2 mL) is added (Scheme A). The reaction mixture is heated for 15 minutes at 100° C. Purification by preparative HPLC (Luna C18 Phenomenex, 5μ, 50×4.6 mm, solvent A: H₂O/0.1% TFA, solvent B: MeCN/0.1% TFA, flow rate 3.0 mL/min, UV: 254 nm, gradient: 20 to 90% B in 15 min) The isolated product (FIG. 1, non-corrected radiochemical yield=19%) is diluted with water (3 mL) and passed through a tC18 SepPak Light cartridge (Waters) that had been activated by flushing with ethanol (5 mL) and water (10 mL). The cartridge is eluted with water (5 mL) and flushed with nitrogen (1 min @ 100 mL/min) Elution with ethanol into a solution of PBS affords ¹⁸F-37 or 37* (50 MBq) with 89% formulation recovery (corrected for decay). QC HPLC (Kinetex C18 Phenomenex, 2.6μ, 50×4.6 mm, solvent A: H₂O/0.1% TFA, solvent B: MeCN/0.1% TFA, flow rate 1.0 mL/min, UV: 254 nm, gradient: 20 to 90% B in 15 min) shows ¹⁸F-37 or 37* with a radiochemical purity of 98% (FIG. 2).

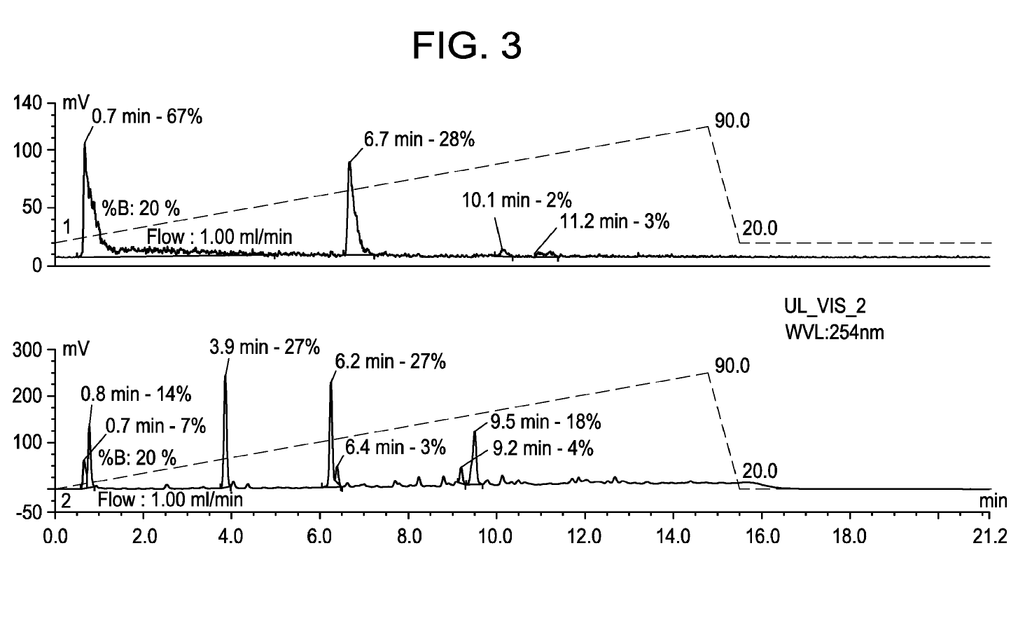

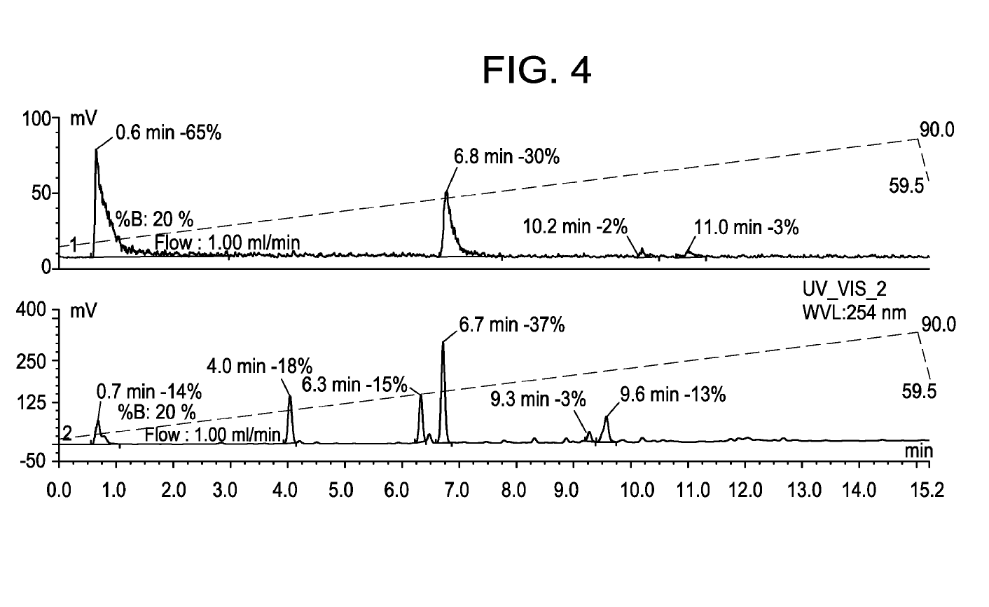

### Example 2

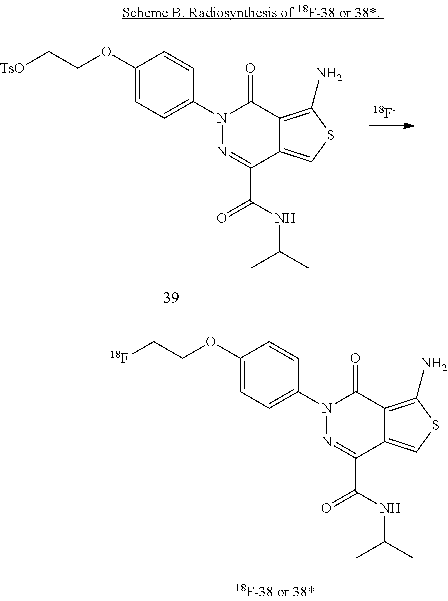

[¹⁸F]Fluoride is azeotropically dried in a Wheaton vial as described in Example 1. The vial is cooled to room temperature and a solution of tosylate 39 (2.0 mg, 3.7 mmol) in anhydrous DMSO (0.2 mL) is added (Scheme B). The reaction mixture is heated for 15 minutes at 100° C. Aliquots of the crude reaction mixture (10 mL) are quenched into HPLC mobile phase (100 mL, 35% solvent B) after 1 min, 5 min, and 15 min Analytical HPLC (Kinetex C18 Phenomenex, 2.6μ, 50×4.6 mm, solvent A: H₂O/0.1% TFA, solvent B: MeCN/0.1% TFA, flow rate 1.0 mL/min, UV: 254 nm, gradient: 20 to 90% B in 15 min) reveals formation of ¹⁸F-38 or 38* (FIG. 3). Injection of cold reference compound confirms the radioactivity signal as product ¹⁸F-38 or 38* (FIG. 4).

### Example 3

Fluorine-18 is produced and azeotropically dried in a Wheaton vial as described in Example 1. Alternative phase-transfer systems such as [¹⁸F]tetrabutylammoniumfluoride hydrogen carbonate (TBAF) and [¹⁸F]F⁻/KHCO₃/Kryptofix are applied with tosylate 39 (2.0 mg, 3.7 mmol) dissolved in anhydrous DMSO (0.2 mL). The reaction mixtures are either heated to 100° C. or irradiated by microwave (50 W, set temperature 90° C.). Table 1 summarizes a time-course study for the radiochemistry optimization.

TABLE 1
Comparison of analytical radiochemical yields of ¹⁸F-38 or 38* under
different reaction conditions.

| | Reaction | 1 min | 5 min | 15 min | 5 s | 10 s | 15 s |
|---|---|---|---|---|---|---|---|
| No. | conditions | 100° C. | 100° C. | 100° C. | MW | MW | MW |
| 1 | K₂CO₃/ | 31% | 26% | 28% | | | |
| | Kryptofix | | | | | | |
| 2 | TBAF | 4% | 10% | 10% | | | |
| 3 | TBAF | | | | 34% | 33% | 36% |
| 4 | KHCO₃/ | | | | 19% | 23% | 20% |
| | Kryptofix | | | | | | |

### Example 4

### Preparation of Compound 57

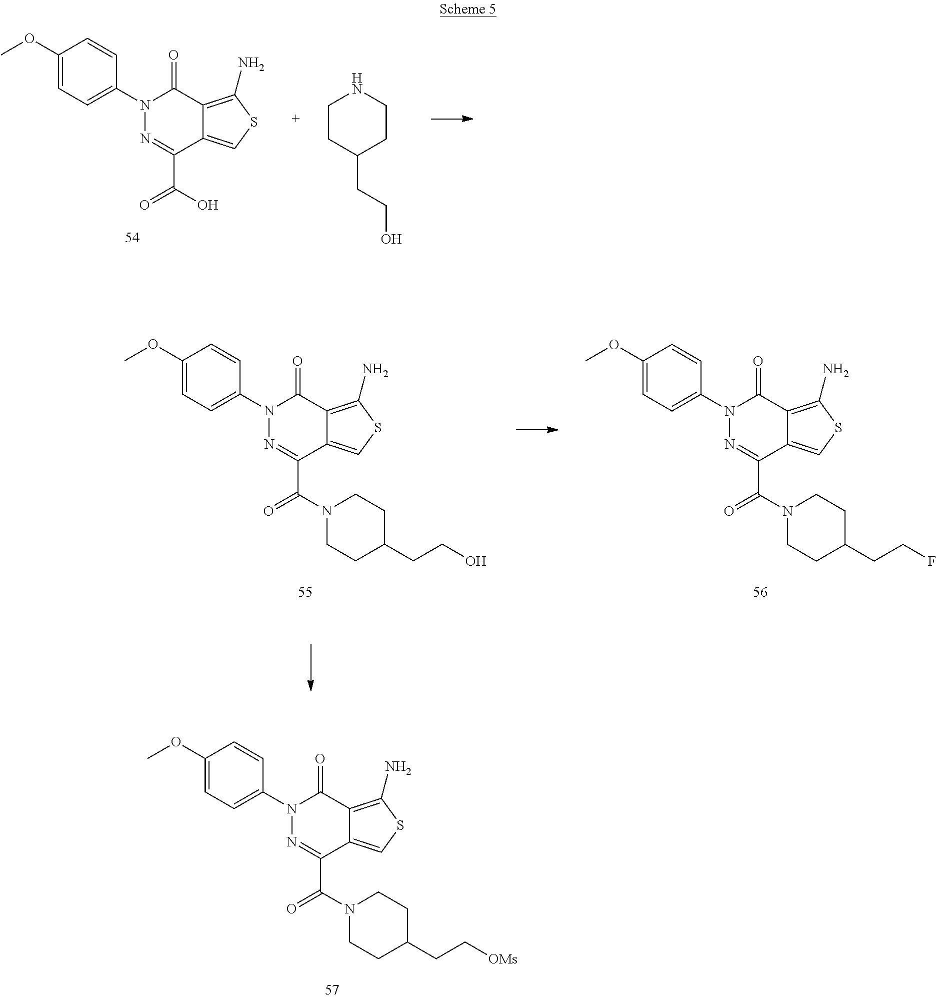 [SMILES: COc1ccc(-n2nc(C(=O)O)c3csc(N)c3c2=O)cc1 ; OCCC1CCNCC1 ; COc1ccc(-n2nc(C(=O)N3CCC(CCO)CC3)c3csc(N)c3c2=O)cc1 ; COc1ccc(-n2nc(C(=O)N3CCC(CCF)CC3)c3csc(N)c3c2=O)cc1 ; COc1ccc(-n2nc(C(=O)N3CCC(CCOS(C)(=O)=O)CC3)c3csc(N)c3c2=O)cc1]

### 4a. Preparation of Compound 55

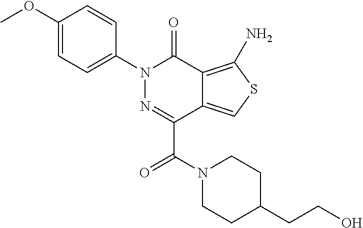 [SMILES: COc1ccc(-n2nc(C(=O)N3CCC(CCO)CC3)c3csc(N)c3c2=O)cc1]

A mixture of 54 (250 mg, 0.788 mmol) (prepared according to Example 13d below), 2-(piperidin-4-yl)ethanol (122 mg, 0.94 mmol), and benzotriazole-1-yl-oxy-tris-(dimethylamino)-phosphonium hexafluorophosphate reagent (523 mg, 1.18 mmol) was dissolved in anhydrous DMSO (10 mL) and DIPEA (204 mg, 1.576 mmol, 0.27 mL) was added to it. The reaction mixture was stirred at room temperature for 16 h. The reaction mixture diluted with water (100 mL) and the resulting mixture was extracted with ethyl acetate (2×100 mL). The organic layer was washed with brine (100 mL), dried (Na₂SO₄), filtered and evaporated under vacuum. The residue was stirred with diethyl ether overnight. The precipitate was filtered and allowed to dry to give 300 mg (85%) 55 as a yellow solid.

LC-MS: m/z calcd for C₂₁H₂₄N₄O₄S, 428, found 429.5 (M+H)⁺

¹H NMR (300 MHz, CDCl₃): δ_{H} 1.3 (2H, m, CH₂CH₂CH), 1.5 (2H, q, J=6 Hz, CHCH₂CH₂), 1.8 (3H, m, CH₂CHCH₂), 2.8 (1H, t, J=15 Hz, NCH₂CH₂), 3.1 (1H, t, J=15 HzNCH₂CH₂), 3.5 (1H, t, J=9 Hz, CH₂OH), 3.72 (2H, t, J=6 Hz, NCH₂CH₂OH), 3.86 (3H, s, ArOCH₃), 4.1 (1H, d, J=15 Hz, NCH₂CH₂), 4.7 (1H, d, J=15 Hz, NCH₂CH₂), 6.66 (1H, s, SCH), 6.98 (2H, d, J=9 Hz, ArCH) and 7.45 (2H, d, J=9 Hz, ArCH).

### 4b. Preparation of Compound 56

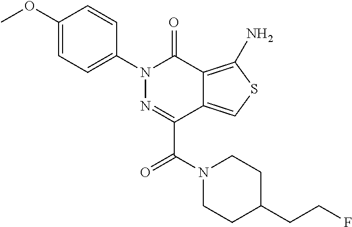 [SMILES: COc1ccc(-n2nc(C(=O)N3CCC(CCF)CC3)c3csc(N)c3c2=O)cc1]

55 (300 mg, 0.7 mmol) was dissolved in anhydrous Chloroform (20 mL) and diethylaminosulfur trifluoride (113 mg, 0.7 mmol) diluted with CHCl₃ (5 mL) was added dropwise at 0 C over 10 min. The reaction was monitored every 10 min by TLC. Thereafter reaction mixture was diluted with excess CHCl₃ (100 mL), washed with saturated NaHCO₃ (20 mL) and extracted with ethyl acetate (2×50 mL). The organic layer was filtered, dried (Na₂SO₄) and concentrated to yield the crude product. The crude product was purified by Semi-prep HPLC using acetonitrile:methanol (50:50) and 20% ammonium acetate (pH 4.3). 1% HCl solution (5 mL) was added to the pooled fractions before freeze-drying to yield 40 mg (13%) as a yellow solid.

LC-MS: m/z calcd for C₂₁H₂₃FN₄O₃S, 430.50, found 431.4 (M+H)⁺

¹H NMR (500 MHz, CDCl₃): δ_{H} 1.6 (7H, m, ringNCH₂CH₂CH₂CHCH₂CH₂F), 2.73 (1H, t, J=15 Hz, NCH₂CH₂), 3.05 (1H, t, J=15 Hz, NCH₂CH₂), 3.77 (3H, s, ArOCH₃), 4.0 (1H, d, J=15 Hz, NCH₂CH₂), 4.4 (1H, t, J=5 Hz, CH₂CH₂F), 4.5 (1H, t, J=5 Hz, CH₂CH₂F), 4.64 (1H, d, J=15 Hz, NCH₂CH₂), 6.58 (1H, s, SCH), 6.89 (2H, d, J=10 Hz, ArCH) and 7.35 (2H, d, J=10 Hz, ArCH).

### 4c. Preparation of Compound 57

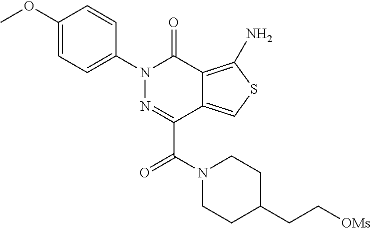 [SMILES: COc1ccc(-n2nc(C(=O)N3CCC(CCOS(C)(=O)=O)CC3)c3csc(N)c3c2=O)cc1]

55 (450 mg, 1.05 mmol) was dissolved in 1:1 mixture of DCM and Dioxane (20 mL) and N,N-Dimethylaminopyridine (256 mg, 2.1 mmol) was added. Methanesulphonyl chloride (120 mg, 1.05 mmol) diluted with dichloromethane (10 mL) was added over a period of 1 h. The reaction mixture was stirred at room temperature for 2 h. Thereafter the reaction mixture was diluted DCM (100 mL), washed with water (2×50 mL) and brine (50 mL). The organic layer was dried over sodium sulfate and concentrated under vacuum to give crude product. The crude product was purified by column chromatography to give 90 mg (17%) of desired product.

LC-MS: m/z calcd for C₂₂H₂₆N₄O₆S₂, 506.60, found 506.9 (M+H)⁺.

¹H NMR (500 MHz, CDCl₃): δ_{H} 1.3 (4H, m, ringNCH₂CH₂CHCH₂CH₂), 1.7 (3H, m, CHCH₂CH₂O Ms), 2.75 (1H, t, J=15 Hz, NCH₂CH₂), 3.0 (3H, s, SO₂CH₃), 3.1 (1H, t, J=15 Hz, NCH₂CH₂), 3.8 (3H, s, ArOCH₃), 4.1 (1H, d, J=15 Hz, NCH₂CH₂), 4.3 (2H, t, J=5 Hz, CH₂CH₂OH), 4.7 (1H, d, J=15 Hz, NCH₂CH₂), 6.6 (1H, s, SCH), 6.9 (2H, d, J=10 Hz, ArCH) and 7.4 (2H, d, J=10 Hz, ArCH).

### Example 5

### Preparation of Compound 59

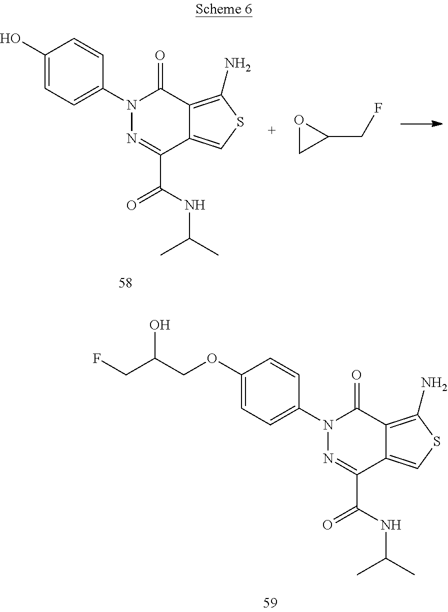 [SMILES: CC(C)NC(=O)c1nn(-c2ccc(O)cc2)c(=O)c2c(N)scc12 ; FCC1CO1 ; CC(C)NC(=O)c1nn(-c2ccc(OCC(O)CF)cc2)c(=O)c2c(N)scc12]

### 5a. Preparation of Compound 59

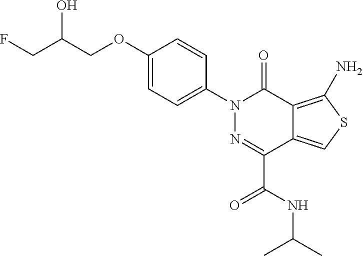 [SMILES: CC(C)NC(=O)c1nn(-c2ccc(OCC(O)CF)cc2)c(=O)c2c(N)scc12]

58 (100 mg, 0.29 mmol) (prepared according to Example 13f), was dissolved in DMF (10 mL), added 1N NaOH solution (17.4 mg, 0.43 mmol) and epifluorohydrin (26 mg, 0.348 mmol). The reaction mixture was stirred at 100 C for 3 h in microwave. Thereafter the reaction mixture was diluted with water and extracted with ethyl acetate (2×100 mL). The combined organic extract was washed with brine (50 mL). The organic layer was dried over sodium sulfate and concentrated under vacuum to give crude product. The crude product was purified by silica gel chromatography to give 32 mg (26%) of the desired product.

LC-MS: m/z calcd for C₁₉H₂₁FN₄O₄S, 420.46, found 420.9 (M+H)⁺.

¹H NMR (500 MHz, CDCl₃): δ_{H} 1.15 (6H, d, J=10 Hz, CH(CH₃)₂), 4.05 (4H, m, ArOCH₂CH & CH(CH₃)₂), 4.46 (1H, m, FCH₂CH), 4.56 (1H, m, FCH₂CH), 5.5 (1H, s, CHOH), 7.04 (2H, d, J=10 Hz, ArCH), 7.18 (1H, s, SCH), 7.5 (2H, d, J=10 Hz, ArCH), 7.55 (2H, s, CHNH₂), 7.91 (1H, d, J=5 Hz, CONH).

### Example 6

### Preparation of compound 60

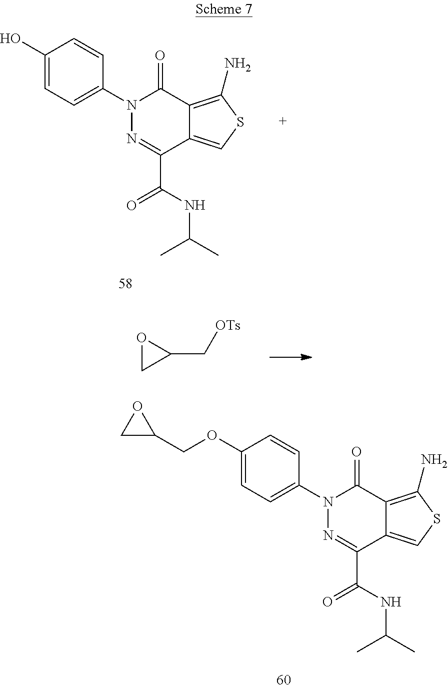

58 (600 mg, 1.74 mmol) (prepared according to Example 13f) was dissolved in DMF (15 mL), Cesium carbonate (848 mg, 2.61 mmol) and glycidyl tosylate (397.3 mg, 1.74 mmol) are added. The reaction mixture was stirred for 15 h at room temperature. Thereafter the reaction mixture was diluted with water and extracted with ethyl acetate (2×150 mL). The combined organic extract was washed with brine (50 mL). The organic layer was dried over sodium sulfate and concentrated under vacuum to give crude product. The crude product was purified by column chromatography to give 80 mg (11%) of the desired product.

LC-MS: m/z calcd for C₁₉H₂₀N₄O₄S, 400.12, found 401.2 (M+H)⁺.

¹H NMR (300 MHz, CDCl₃): δ_{H} 1.22 (6H, d, J=6 Hz, CH(CH₃)₂), 2.78 (1H, m, OCH₂CH), 2.93 (1H, t, J=6 Hz, OCH₂CH), 3.38 (1H, m, CH₂CHCH₂), 4.0 (1H, m, ArOCH₂), 4.2 (1H, m, CH(CH₃)₂), 4.3 (1H, dd, J1=9 Hz, J2=3 Hz, ArOCH₂), 6.15 (2H, s, CNH₂), 6.9 (1H, d, J=9 Hz, CHNH), 7.02 (2H, d, J=9 Hz, ArCH), 7.44 (2H, d, J=9 Hz, ArCH) and 7.58 (1H, s, SCH).

### Example 7

### Preparation of Compound 61

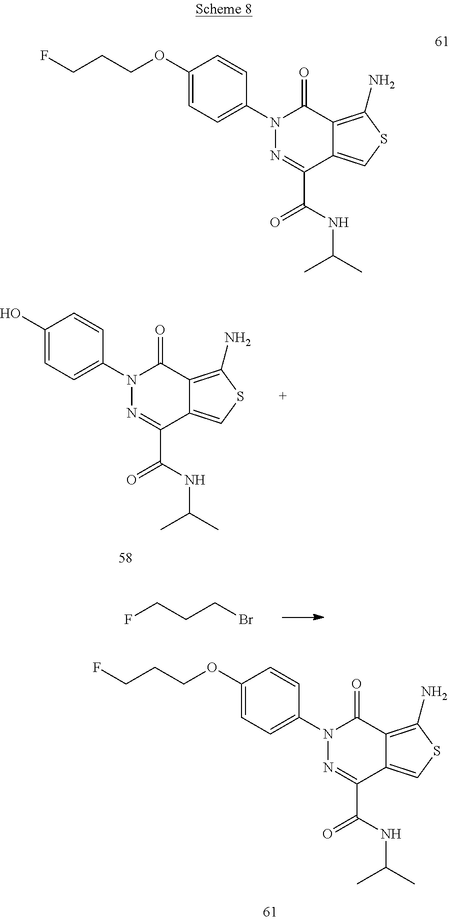 [SMILES: CC(C)NC(=O)c1nn(-c2ccc(OCCCF)cc2)c(=O)c2c(N)scc12 ; CC(C)NC(=O)c1nn(-c2ccc(O)cc2)c(=O)c2c(N)scc12 ; FCCCBr ; CC(C)NC(=O)c1nn(-c2ccc(OCCCF)cc2)c(=O)c2c(N)scc12]

58 (400 mg, 1.16 mmol) (prepared according to Example 130 was dissolved in DMF (15 mL), cesium carbonate (568 mg, 1.74 mmol) and bromofluoropropane (162 mg, 1.16 mmol) are added. The reaction mixture was stirred for 15 h. Thereafter the reaction mixture was diluted with water and extracted with ethyl acetate (2×150 mL). The combined organic extract was washed with brine (50 mL). The organic layer was dried over sodium sulfate and concentrated under vacuum to give crude product. The crude product was purified by column chromatography to give 85 mg (18%) of 98.3% of the desired product.

LC-MS: m/z calcd for C₁₉H₂₁FN₄O₃S, 404.46, found 405.2 (M+H)⁺.

¹H NMR (300 MHz, CDCl₃): δ_{H} 1.22 (6H, d, J=6 Hz, CH(CH₃)₂), 2.15 (1H, q, J=6 Hz, CH₂CH₂CH₂), 2.24 (1H, q, J=6 Hz, CH₂CH₂CH₂), 4.15 (3H, m, ArOCH₂& CH(CH₃)₂), 4.58 (1H, t, J=6 Hz, FCH₂), 4.74 (1H, t, J=6 Hz, FCH₂), 6.2 (2H, s, CNH₂), 6.9 (1H, d, J=9 Hz, CHNH), 7.0 (2H, d, J=9 Hz, ArCH), 7.42 (2H, d, J=9 Hz, ArCH) and 7.57 (1H, s, SCH).

### Example 8

### Preparation of Compound 62

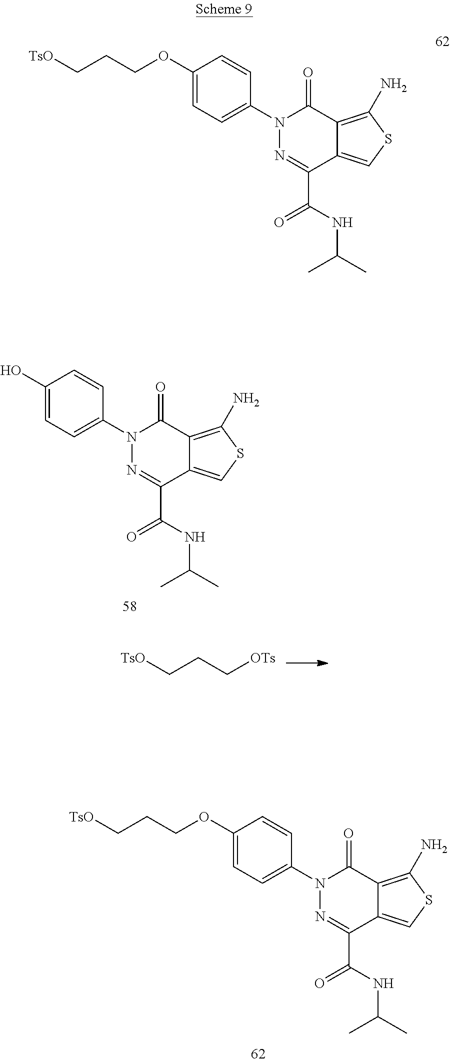 [SMILES: Cc1ccc(S(=O)(=O)OCCCOc2ccc(-n3nc(C(=O)NC(C)C)c4csc(N)c4c3=O)cc2)cc1 ; CC(C)NC(=O)c1nn(-c2ccc(O)cc2)c(=O)c2c(N)scc12 ; Cc1ccc(S(=O)(=O)OCCCOS(=O)(=O)c2ccc(C)cc2)cc1 ; Cc1ccc(S(=O)(=O)OCCCOc2ccc(-n3nc(C(=O)NC(C)C)c4csc(N)c4c3=O)cc2)cc1]

58 (300 mg, 0.87 mmol) (prepared according to Example 13f) was dissolved in DMF (10 mL), cesium carbonate (283 mg, 0.87 mmol and 1,3-Propanediol di-p-tosylate (335 mg, 0.87 mmol) are added. The reaction mixture was stirred for 15 h. Thereafter the reaction mixture was diluted with water and extracted with ethyl acetate (2×150 mL). The combined organic extract was washed with brine (50 mL). The organic layer was dried over sodium sulfate and concentrated under vacuum to give crude product. The crude product was purified by column chromatography to give 110 mg (23%) of the desired product.

LC-MS: m/z calcd for C₂₆H₂₈N₄O₆S₂, 556.65, found 557.2 (M+H)⁺.

¹H NMR (500 MHz, DMSO-d6): δ_{H} 1.15 (6H, d, J=5 Hz, CH(CH₃)₂), 2.06 (2H, q, J=5 Hz, CH₂CH₂CH₂), 2.38 (3H, s, ArCH₃), 3.96 (2H, t, J=5 Hz, SO₃CH₂), 4.06 (1H, m, CH(CH₃)₂), 4.22 (2H, t, J=5 Hz, ArOCH₂), 6.9 (2H, d, J=10 Hz, ArCH), 7.19 (1H, s, SCH), 7.42 (2H, d, J=10 Hz, ArCH), 7.48 (2H, d, J=10 Hz, TsCH), 7.55 (2H, s, NH₂), 7.78 (2H, d, J=10 Hz, TsCH) and 7.88 (1H, d, J=5 Hz, NHCH).

### Example 9

### Preparation of Compound 64

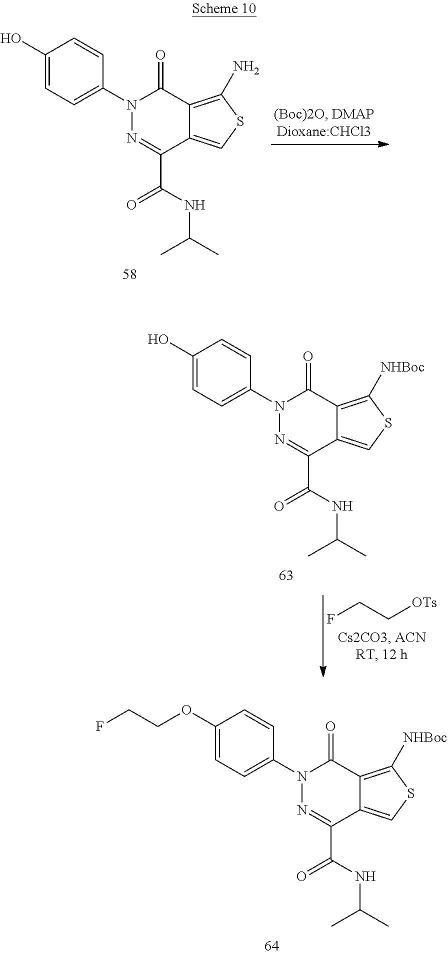 [SMILES: CC(C)NC(=O)c1nn(-c2ccc(O)cc2)c(=O)c2c(N)scc12 ; CC(C)NC(=O)c1nn(-c2ccc(O)cc2)c(=O)c2c(NC(=O)OC(C)(C)C)scc12 ; Cc1ccc(S(=O)(=O)OCCF)cc1 ; CC(C)NC(=O)c1nn(-c2ccc(OCCF)cc2)c(=O)c2c(NC(=O)OC(C)(C)C)scc12]

### 9a. Preparation of Compound 63

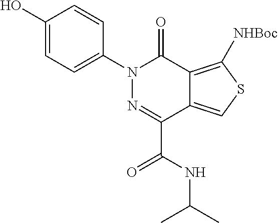 [SMILES: CC(C)NC(=O)c1nn(-c2ccc(O)cc2)c(=O)c2c(NC(=O)OC(C)(C)C)scc12]

58 (400 mg, 1.16 mmol) (prepared according to Example 13f) was dissolved in a 1:1 mixture of dioxane and chloroform (25 mL) and dimethyl amino pyridine (221 mg, 1.74 mmol), di-tert-butyl dicarbonate (253 mg, 1.16 mmol) were added. The reaction mixture was stirred for 2 h. Thereafter the reaction mixture was diluted with water and extracted with dichloromethane (2×150 mL). The combined organic extract was washed with brine (50 mL). The organic layer was dried over sodium sulfate and concentrated under vacuum to give crude product. The crude product was purified by column chromatography to give 300 mg (58%) of the desired product.

LC-MS: m/z calcd for C₂₁H₂₄N₄O₅S, 444.50, found 444.3 (M+)⁺.

¹H NMR (300 MHz, CDCl₃): δ_{H} 1.25 (6H, d, J=6 Hz, CH(CH₃)₂), 1.60 (9H, s, NH(CH₃)₃), 4.2 (1H, m, CH(CH₃)₂), 6.92 (1H, d, J=9 Hz, NHCH), 7.3 (2H, d, J=9 Hz, ArCH), 7.55 (2H, d, J=9 Hz, ArCH) and 7.61 (1H, s, SCH).

### 9b. Preparation of Compound 64

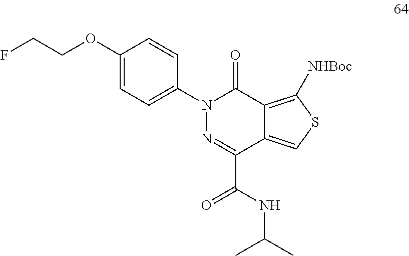 [SMILES: CC(C)NC(=O)c1nn(-c2ccc(OCCF)cc2)c(=O)c2c(NC(=O)OC(C)(C)C)scc12]

63 (30 mg, 0.067 mmol) was dissolved in Acetonitrile (15 mL),added cesium carbonate (33 mg, 0.101 mmol) and fluoroethyl tosylate (15 mg, 0.067 mmol) are added. The reaction mixture was stirred for 15 h. Thereafter the reaction mixture was diluted with water and extracted with ethyl acetate (2×150 mL). The combined organic extract was washed with brine (50 mL). The organic layer was dried over sodium sulfate and concentrated under vacuum to give crude product. The crude product was purified by column chromatography to give 10 mg of the desired product.

LC-MS: m/z calcd for C₂₃H₂₇FN₄O₅S, 490.55, found 491.0 (M+)⁺.

¹H NMR (500 MHz, CDCl₃): δ_{H} 1.2 (6H, d, J=5 Hz, CH(CH₃)₂), 1.45 (9H, s, O(CH₃)₃), 4.15 (1H, m, CH(CH₃)₂), 4.2 (1H, t, J=5 Hz, ArOCH₂), 4.24 (1H, t, J=5 Hz, ArOCH₂), 4.68 (1H, t, J=5 Hz, FCH₂), 4.78 (1H, t, J=5 Hz, FCH₂), 6.90 (1H, d, J=10 Hz, NHCH), 7.0 (2H, d, J=10 Hz, ArCH), 7.40 (2H, d, J=10 Hz, ArCH), 8.06 (1H, s, SCH) and 10.0 (1H, s, NHBoc).

### Example 10

### Preparation of Compound 67

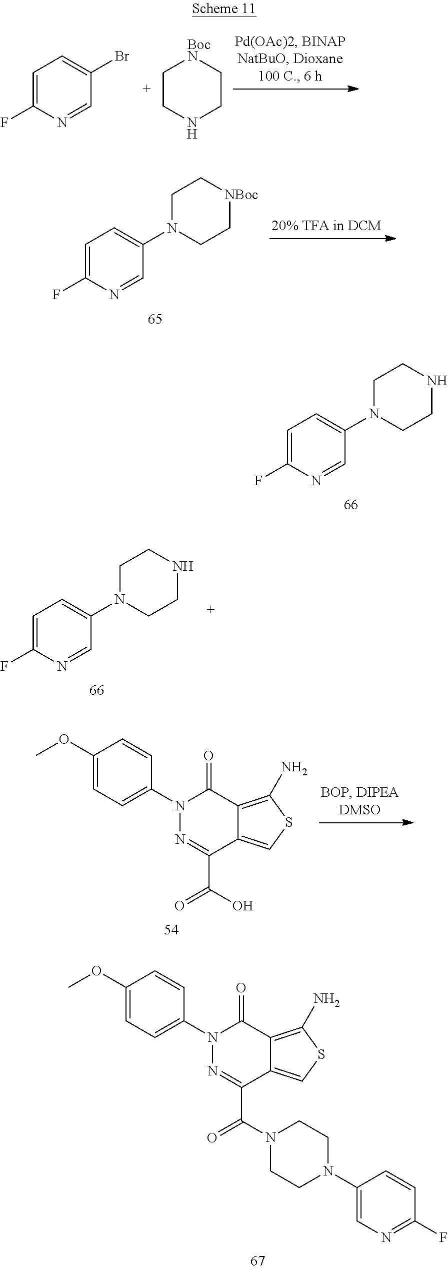

### 10a. Preparation of Compound 65

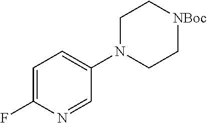 [SMILES: CC(C)(C)OC(=O)N1CCN(c2ccc(F)nc2)CC1]

5-bromo-2-fluoropyridine (1.5 g, 8.52 mmol) and sodium-tert-butoxide (1.22 g, 12.79 mmol) were dissolved in 1,4-Dioxane (30 mL), was added tert-butyl piperazine-1-carboxylate (1.58 g, 8.52 mmol), nitrogen gas was purged through the reaction mixture for 5 min, was added BINAP (0.318 g, 0.511 mmol) followed by Palladium(II)acetate (0.038 g, 0.17 mmol). The reaction mixture was stirred under reflux for 6 h. Thereafter the reaction mixture was diluted with water and extracted with ethyl acetate (2×200 mL). The combined organic extract was washed with brine (50 mL). The organic layer was dried over sodium sulfate and concentrated under vacuum to give crude product. The crude product was purified by column chromatography to give 1.0 g of the desired product.

LC-MS: m/z calcd for C₁₄H₂₀FN₃O₂, 281.33, found 281.9 (M+H)⁺.

¹H NMR (500 MHz, CDCl₃): δ_{H} 1.4 (9H, s, O(CH₃)₃), 3.0 (4H, t, J=5 Hz, NCH₂CH₂N), 3.5 (4H, t, J=5 Hz, NCH₂CH₂N), 6.80 (1H, m, ArCH), 7.3 (1H, m, ArCH) and 7.7 (1H, s, ArCH).

### 10b. Preparation of Compound 66

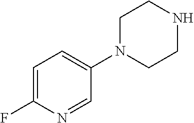 [SMILES: Fc1ccc(N2CCNCC2)cn1]

65 (650 mg, 2.31 mmol) was dissolved in dichloromethane (10 mL) and cooled to 5 C. 5 mL solution of 20% TFA in DCM was added dropwise to the reaction mass. The reaction mixture was stirred for 3 h. Thereafter the reaction mixture was diluted with excess DCM (100 mL) and washed with water wash (2×50 mL) followed brine (50 mL). The organic layer was dried over sodium sulfate and concentrated under vacuum to give 500 mg of the desired product.

LC-MS: m/z calcd for C₉H₁₂FN₃, 181.21, found 181.7 (M+H)⁺.

¹H NMR (500 MHz, DMSO-d₆): δ_{H} 3.26 (4H, m, NCH₂CH₂N), 3.38 (4H, m, NCH₂CH₂N), 7.1 (1H, m, ArCH), 7.7 (1H, m, ArCH), 7.9 (1H, s, ArCH) and 9.0 (1H, s, NH)

### 10c. Preparation of Compound 67

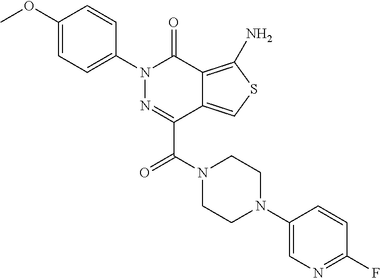 [SMILES: COc1ccc(-n2nc(C(=O)N3CCN(c4ccc(F)nc4)CC3)c3csc(N)c3c2=O)cc1]

A mixture of 54 (300 mg, 0.945 mmol), 66(171 mg, 0.945 mmol) and benzotriazole-1-yl-oxy-tris-(dimethylamino)-phosphonium hexafluorophosphate reagent (627 mg, 1.418 mmol) in anhydrous DMSO (10 mL) was added DIPEA (244 mg, 1.891 mmol). The reaction mixture was stirred at room temperature for 16 h. The progress of the reaction mass was monitored by LCMS. The reaction mixture diluted with water (100 mL) and the resulting mixture was extracted with ethyl acetate (2×100 mL). The organic layer was washed with brine (100 ml), dried (Na₂SO₄), filtered, and evaporated under vacuum. The residue was stirred with diethyl ether overnight. The precipitate was filtered and allowed to dry to give 60 mg (13%) of 67 as a yellow solid.

LC-MS: m/z calcd for C₂₃H₂₁FN₆O₃S, 480.51, found 481.0 (M+H)⁺.

¹H NMR (500 MHz, DMSO-d₆): δ_{H} 3.1 (2H, s, NCH₂CH₂N), 3.35 (2H, m, NCH₂CH₂N), 3.7 (2H, s, NCH₂CH₂N), 3.8 (5H, s, NCH₂CH₂N & ArOCH₃), 6.6 (1H, s, SCHCH), 6.98 (2H, d, J=10 Hz, ArCH), 7.05 (1H, d, J=5 Hz, ArCH), 7.4 (2H, d, J=5 Hz, ArCH), 7.55 (2H, s, NH₂), 7.60 (1H, s, ArCH) and 7.85 (1H, s, ArCH).

### Example 11

### Preparation of Compound 70

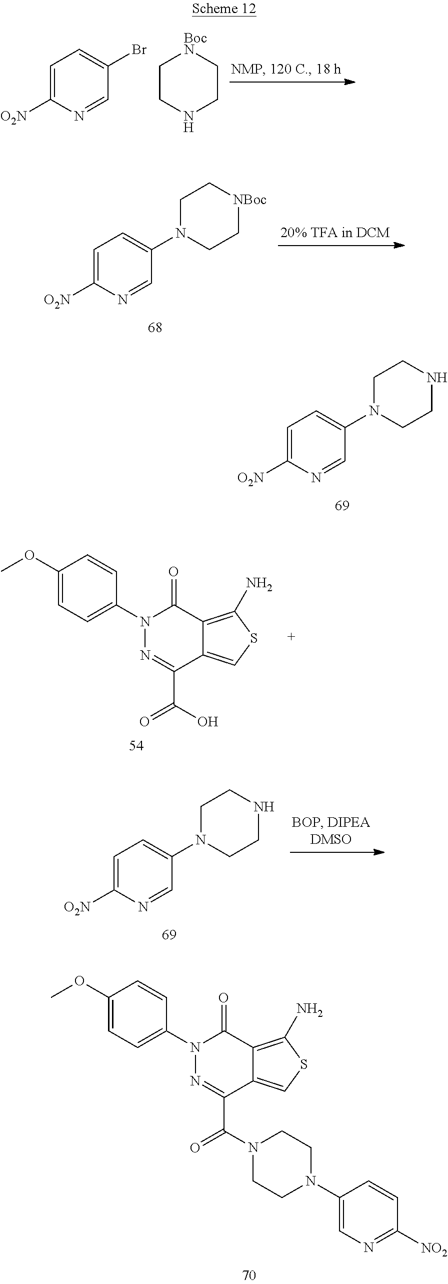

### 11a. Preparation of Compound 68

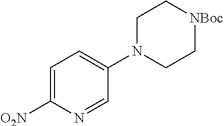 [SMILES: CC(C)(C)OC(=O)N1CCN(c2ccc([N+](=O)[O-])nc2)CC1]

Dissolved 5-bromo-2-nitropyridine (1.0 g, 4.92 mmol) and tert-butyl piperazine-1-carboxylate (1.1 g, 5.91 mmol) in N-methylpyrrolidine and stirred at 120 C for 18 h. Thereafter the reaction mixture was cooled to 30 C and diluted with water and extracted with ethyl acetate (2×200 mL). The combined organic extract was washed with brine (50 mL). The organic layer was dried over sodium sulfate and concentrated under vacuum to give crude product. The crude product was purified by column chromatography to give 400 mg of the desired product.

LC-MS: m/z calcd for C₁₄H₂₀N₄O₄, 308.33, no ionization

¹H NMR (500 MHz, CDCl₃): δ_{H} 1.4 (9H, s, O(CH₃)₃), 3.38 (4H, t, J=5 Hz, NCH₂CH₂N), 3.58 (4H, t, J=5 Hz, NCH₂CH₂N), 7.14 (1H, dd, J1=5 Hz, J2=10 HZ, ArCH), 8.06 (1H, d, J=5 Hz, ArCH) and 8.11 (1H, d, J=10 Hz, ArCH).

### 11b. Preparation of Compound 69

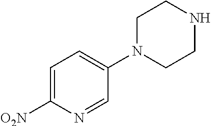 [SMILES: O=[N+]([O-])c1ccc(N2CCNCC2)cn1]

68 (400 mg, 1.29 mmol) was dissolved in dichloromethane (10 mL), cooled to 5 C. 5 mL solution of 20% TFA in DCM was added dropwise. The reaction mixture was stirred for 3 h. Thereafter the reaction mixture was diluted with excess DCM (100 mL) and washed with water (2×50 mL) followed brine (50 mL). The organic layer was dried over sodium sulfate and concentrated under vacuum to give 200 mg (74%) of the desired product.

LC-MS: m/z calcd for C9H12N4O2, 208.22, found 208.7 (M+H)⁺

### 11c. Preparation of Compound 70

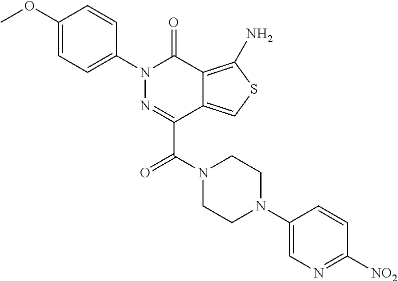 [SMILES: COc1ccc(-n2nc(C(=O)N3CCN(c4ccc([N+](=O)[O-])nc4)CC3)c3csc(N)c3c2=O)cc1]

A mixture of 54 (305 mg, 0.96 mmol), 69 (200 mg, 0.96 mmol) and benzotriazole-1-yl-oxy-tris-(dimethylamino)-phosphonium hexafluorophosphate reagent (637 mg, 1.44 mmol) in anhydrous DMSO (10 mL) was added DIPEA (0.67 mL, 3.84 mmol). The reaction mixture was stirred at room temperature for 12 h. The progress of the reaction mass was monitored by LCMS. The reaction mixture diluted with water (100 mL) and the resulting mixture was extracted with ethyl acetate (2×100 mL). The organic layer was washed with brine (100 ml), dried (Na₂SO₄), filtered, and evaporated under vacuum. The crude product was purified by column chromatography to give 40 mg of the desired product.

LC-MS: m/z calcd for C₂₃H₂₁N₇O₅S, 507.52, found: 508.0 (M+H)⁺.

¹H NMR (500 MHz, DMSO-d₆): δ_{H} 3.55 (2H, s, NCH₂CH₂N), 3.7 (2H, s, NCH₂CH₂N), 3.75 (2H, s, NCH₂CH₂N), 3.8 (5H, s, NCH₂CH₂N & ArOCH₃), 6.74 (1H, s, SCHCH), 6.98 (2H, d, J=10 Hz, ArCH), 7.4 (2H, d, J=10 Hz, ArCH), 7.5 (1H, d, J=10 Hz, ArCH), 7.56 (2H, s, NH₂), 8.18 (1H, d, J=10 Hz, ArCH) and 8.26 (1H, d, J=5 Hz, ArCH).

### Example 12

### Preparation of Compound 74

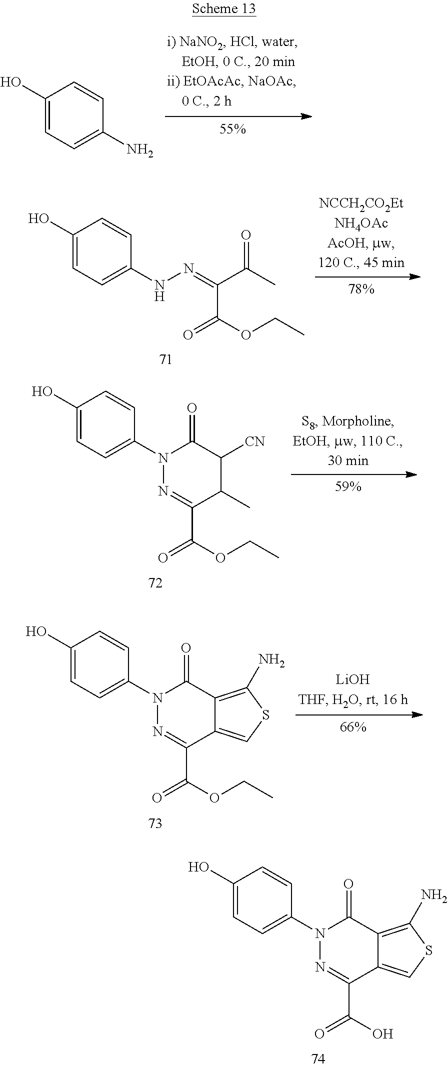 [SMILES: Nc1ccc(O)cc1 ; CCOC(=O)/C(=N\Nc1ccc(O)cc1)C(C)=O ; CCOC(=O)C1=NN(c2ccc(O)cc2)C(=O)C(C#N)C1C ; CCOC(=O)c1nn(-c2ccc(O)cc2)c(=O)c2c(N)scc12 ; Nc1scc2c(C(=O)O)nn(-c3ccc(O)cc3)c(=O)c12]

### 12a. Preparation of Compound 71

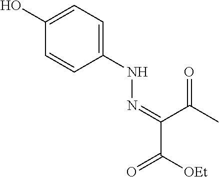 [SMILES: CCOC(=O)/C(=N/Nc1ccc(O)cc1)C(C)=O]

4-Aminophenol (5 g, 45.87 mmol) was dissolved in a mixture of 37% hydrochloric acid (7 mL), ethanol (15 mL) and water (20 mL). The reaction mixture was cooled to 0 C in an ice-water bath before a solution of sodium nitrite (3.21 g, 45.87 mmol) in water (10 mL) was added dropwise. The resulting mixture was stirred at 0 C for 20 min Sodium acetate (24.95 g, 183.49 mmol) in water (50 mL) and ethyl acetoacetate (5.96 g, 45.87 mmol, 5.84 mL) were added and the reaction mixture was stirred at 0 C for 2 h. The precipitated solid was filtered, washed with water, and dried under high vacuum to provide 6 g (52%) of the desired product as brown solid.

LC-MS: m/z calcd for C₁₂H₁₄N₂O₄, 250.1, found 250.6 (M+H)⁺.

¹H NMR (500 MHz, MeOD): δ_{H} 1.35-1.41 (3H, m, OCH₃), 2.47 (3H, s, CH₂CH₃), 4.30-4.39 (2H, m, O—CH₂), 6.82-6.88 (2H, m, phenyl-3H and 5H) and 7.30-7.39 (2H, m, phenyl-2H and 6H)

### 12b. Preparation of Compound 72

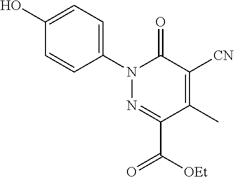 [SMILES: CCOC(=O)c1nn(-c2ccc(O)cc2)c(=O)c(C#N)c1C]

A mixture of 71 (2.0 g, 8 mmol), ethyl cyanoacetate (1.81 g, 16 mmol, 1.70 mL) and ammonium acetate (2.46 g, 31.91 mmol) in acetic acid (6 mL) was heated in microwave at 120 C for 45 min. The resulting mixture was diluted with water (50 mL) and extracted with ethyl acetate (3×100 mL). The combined organic extract was washed with water (30 mL), brine (30 mL), dried over sodium sulfate and evaporated under vacuum. The crude compound was washed with hexane (3×50 mL) and filtered to obtain 1.8 g (78%) as brown solid.

LC-MS: m/z calcd for C₁₅H₁₃N₃O₄ 299.0, found 298.5 (M−H)⁻.

¹H NMR (500 MHz, CD3CN): δ_{H} 1.26 (3H, t, J=5 Hz, CH₂CH₃), 2.59 (3H, s, CH₃), 4.29 (2H, q, J=10 Hz, CH₂CH₃), 6.87 (2H, d, J=5 Hz, phenyl-2H and 6H) and 7.30 (2H, d, J=5 Hz, phenyl-3H and 5H).

### 12c. Preparation of Compound 73

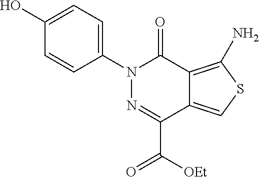 [SMILES: CCOC(=O)c1nn(-c2ccc(O)cc2)c(=O)c2c(N)scc12]

A mixture of 71 (2 g, 6.38 mmol), sulfur (0.30 g, 9.24 mmol), and morpholine (1.1 g, 12.67 mmol, 1.1 mL) in ethanol (6 mL) was heated in microwave to 120 C for 30 min After the mixture was cooled, the precipitate formed was filtered. Recrystallization from hot ethanol yielded 1.3 g (59%) as a pale brown solid.

LC-MS: m/z calcd for C₁₅H₁₃N₃O₄S, 331.0, found 331.9 (M+H)⁺.

¹H NMR (500 MHz, DMSO): δ_{H} 1.29 (3H, t, J=5 Hz, CH₂CH₃), 4.31 (2H, q, J=10 Hz, CH₂CH₃), 6.83 (2H, d, J=5 Hz, phenyl-2H and 6H), 7.08 (1H, s, SCH), 7.26 (2H, d, J=5 Hz, phenyl-3H and 5H) and 7.59 (2H, s, NH₂).

### 12d. Preparation of Compound 74

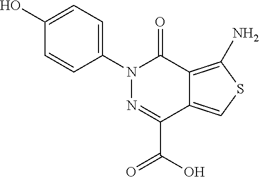 [SMILES: Nc1scc2c(C(=O)O)nn(-c3ccc(O)cc3)c(=O)c12]

Lithium hydroxide monohydrate (0.1 g, 4.33 mmol) was added to a solution of 73 (2 g, 6.03 mmol) in tetrahydrofuran (20 mL) and water (20 mL). The reaction mixture was stirred at room temperature for 16 h. The progress of the reaction was monitored by LCMS. Thereafter the pH of the reaction mass was adjusted to 6 using 1 N HCl and the aqueous layer was extracted with ethyl acetate (3×50 mL). The combined organic layers were separated, dried over sodium sulfate and evaporated to yield 1.2 g (66%) of desired product as a yellow solid.

LC-MS: m/z calcd for C₁₃H₉N₃O₄S 303.3, found 303.9 (M+H)⁺.

¹H NMR (500 MHz, DMSO): δ_{H} 3.16 (1H, s, OH), 6.80 (2H, d, J=5 Hz, phenyl-2H and 6H), 7.14 (1H, s, SCH), 7.22 (2H, d, J=5 Hz, phenyl-3H and 5H) and 7.35 (2H, s, NH₂).

### Example 13

### Preparation of Compound 79

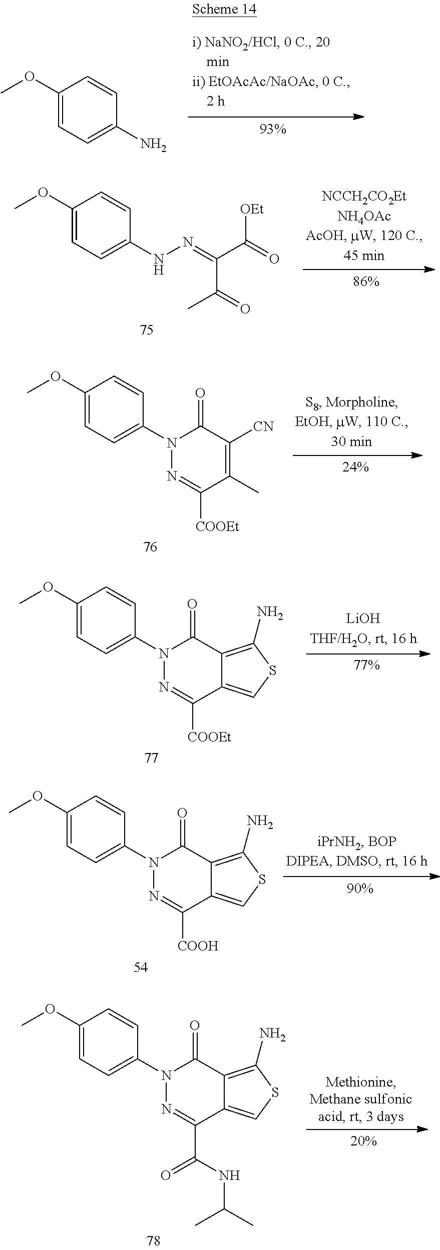

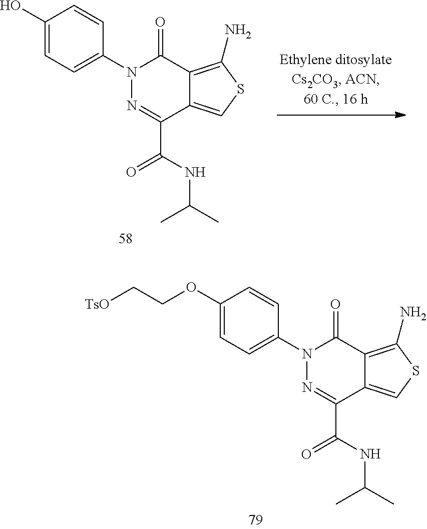 [SMILES: CC(C)NC(=O)c1nn(-c2ccc(O)cc2)c(=O)c2c(N)scc12 ; Cc1ccc(S(=O)(=O)OCCOc2ccc(-n3nc(C(=O)NC(C)C)c4csc(N)c4c3=O)cc2)cc1]

### 13a. Preparation of Compound 75

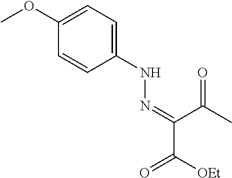 [SMILES: CCOC(=O)/C(=N/Nc1ccc(OC)cc1)C(C)=O]

p-Anisidine (2 g, 16.23 mmol) was dissolved in a mixture of 37% hydrochloric acid (3 mL), ethanol (5 mL) and water (3 mL). The reaction mixture was cooled to 0 C in an ice-water bath before a solution of sodium nitrite (1.12 g, 16.23 mmol) in water (7 mL) was added in dropwise. The resulting mixture was stirred at 0 C for 20 min. Sodium acetate (8.61 g, 63.27 mmol) in water (20 mL) and ethyl acetoacetate (2.1 g, 16.13 mmol, 2 mL) were added and the reaction mixture was stirred at 0 C for 2 h. Then, the precipitated solid was filtered, washed with water and dried under high vacuum to provide 4 g (93%) as yellow solid.

LC-MS: m/z calcd for C₁₃H₁₆N₂O₄ 264.1, found 265.1 (M+H)⁺.

¹H NMR (500 MHz, CDCl₃): δ_{H} 1.32 (3H, t, J=5 Hz, CH₂CH₃), 2.51 (2H, s, OCH₃), 3.75 (3H, s, phenyl-OCH₃), 4.22-4.32 (2H, m, CH₂CH₃), 6.84-6.88 (2H, m, phenyl-3H and 5H), 7.20-7.25 (1H, m, phenyl-2H) and 7.29-7.32 (1H, m, phenyl-5H).

### 13b. Preparation of Compound 76

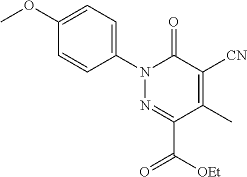 [SMILES: CCOC(=O)c1nn(-c2ccc(OC)cc2)c(=O)c(C#N)c1C]

A mixture of 75 (2.0 g, 7.57 mmol), ethyl cyanoacetate (1.71 g, 15.11 mmol, 1.61 mL) and ammonium acetate (2.33 g, 30.22 mmol) in acetic acid (5 mL) was irradiated in microwave at 120 C for 45 min. The resulting mixture was diluted with water (100 mL) and extracted with ethyl acetate (3×100 mL). The combined organic extract was washed with water (30 mL), brine (30 mL), dried (Na₂SO₄) and evaporated under vacuum. The crude compound was heated in ethanol and filtered hot to yield 2 g (86%) as a dark yellow solid.

LC-MS: m/z calcd for C₁₆H₁₅N₃O₄ 313.3, found 312.5 (M−H)⁺.

¹H NMR (500 MHz, CDCl₃): δ_{H} 1.39 (3H, t, J=5 Hz, CH₂CH₃), 2.74 (3H, s, CNCCCH₃), 3.85 (3H, s, OCH₃), 4.41 (2H, q, J=10 Hz, CH₂CH₃), 6.99 (2H, d, J=5 Hz, phenyl-3H and 5H) and 7.55 (2H, d, J=5 Hz, phenyl-2H and 4H).

### 13c. Preparation of Compound 77

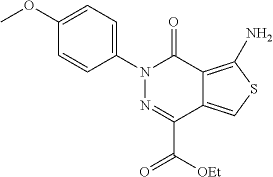 [SMILES: CCOC(=O)c1nn(-c2ccc(OC)cc2)c(=O)c2c(N)scc12]

A mixture of 76 (2 g, 6.38 mmol), sulfur (0.30 g, 9.24 mmol), and morpholine (1.1 g, 12.67 mmol, 1.1 mL) in ethanol (7 mL) was heated to 130 C in microwave for 25 min. The progress of the reaction mass was monitored by HPLC. Thereafter the mixture was cooled and the precipitate formed was filtered. The crude product was recrystallized from ethanol to give 0.530 g (24%) of product as a pale brown solid.

LC-MS: m/z calcd for C₁₆H₁₅N₃O₄S, 345.0, found 346.0 (M+H)⁺.

¹H NMR (500 MHz, CDCl₃): δ_{H} 1.44 (3H, t, J=5 Hz, CH₂CH₃), 3.87 (3H, s, OCH₃), 4.46 (2H, q, J=10 Hz, CH₂CH₃), 6.21 (1H, s, NH₂), 7.01 (2H, d, J=5 Hz, phenyl-3H and 5H), 7.30 (1H, s, SCH) and 7.51 (2H, d, J=5 Hz, phenyl-2H and 4H).

### 13d. Preparation of Compound 54

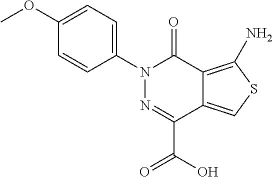 [SMILES: COc1ccc(-n2nc(C(=O)O)c3csc(N)c3c2=O)cc1]

Lithium hydroxide monohydrate (0.1 g, 4.33 mmol) was added to a solution of 77 (0.50 g, 1.44 mmol) in tetrahydrofuran (10 mL) and water (10 mL). The reaction mixture was stirred at room temperature for 16 h. The progress of the reaction mass was monitored by HPLC. Thereafter pH of the reaction mass was adjusted to 6 using 1 N HCl and was extracted with ethyl acetate (3×50 mL). The combined organic layers were separated, dried (Na₂SO₄) and evaporated to yield 0.35 g (77%) of desired product as a yellow solid.

LC-MS: m/z calcd for C₁₄H₁₁N₃O₄S, 317.3, found 318.6 (M+H)⁺.

¹H NMR (500 MHz, MeOD): δ_{H} 3.86 (3H, s, OCH₃), 6.80 (2H, s, NH₂), 7.02 (2H, d, J=5 Hz, phenyl-3H and 5H), 7.20 (1H, s, SCH) and 7.46 (2H, d, J=5 Hz, phenyl-2H and 4H).

### 13e. Preparation of Compound 78

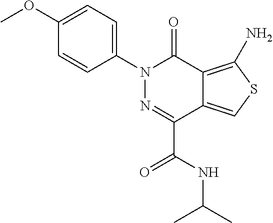 [SMILES: COc1ccc(-n2nc(C(=O)NC(C)C)c3csc(N)c3c2=O)cc1]

A mixture of 54 (0.35 g, 1.10 mmol), iso-propylamine (0.13 g, 2.19 mmol, 0.18 mL), and benzotriazole-1-yl-oxy-tris-(dimethylamino)-phosphonium hexafluorophosphate reagent (0.73 g, 1.65 mmol) in anhydrous DMSO (5 mL) was added DIPEA (0.28 g, 2.16 mmol, 0.38 mL). The reaction mixture was stirred at room temperature for 16 h. The progress of the reaction mass was monitored by LCMS. The reaction mixture diluted with water (25 mL) and the resulting mixture was extracted with dichlormethane (3×75 mL). The organic layer was washed with brine (20 mL), dried (Na₂SO₄), filtered, and evaporated under vacuum. The residue was stirred with diethyl ether overnight. The precipitate was filtered and allowed to dry to yield 0.30 g (90%) as a brown solid.

LC-MS: m/z calcd for C₁₇H₁₈N₄O₃S, 358.1, found 359.1. (M+H)⁺.

¹H NMR (500 MHz, CDCl₃): δ_{H} 1.23 (6H, d, J=5 Hz, (CH₃)₂CH3.84 (3H, s, OCH₃), 4.16-4.23 (1H, m, (CH₃)₂CH), 6.91-7.08 (5H, m, phenyl-3H and 5H, SCH, NH₂) and 7.40-7.46 (2H, m, phenyl-2H and 4H).

### 13f. Preparation of Compound 58

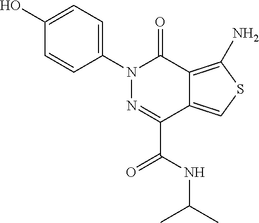 [SMILES: CC(C)NC(=O)c1nn(-c2ccc(O)cc2)c(=O)c2c(N)scc12]

To 78 (0.22 g, 0.61 mmol), methane sulfonic acid (4 mL) and methionine (0.27 g, 1.81 mmol) were added and the reaction mixture was stirred for 3 days. The progress of the reaction mass was monitored by LCMS. Thereafter the reaction mass was poured into ice and the precipitated solid was recovered by centrifugation. The product was dissolved in ethyl acetate and was washed with aqueous bicarbonate solution (50 mL). The organic layer was separated, dried (Na₂SO₄), filtered and evaporated under vacuum to yield 160 mg (80%) as a brown solid.

LC-MS: m/z calcd for C₁₆H₁₆N₄O₃S, 344.0, found 345.0 (M+H)⁺.

¹H NMR (500 MHz, CDCl₃): δ_{H} 1.14 (6H, d, J=5 Hz, 2×(CH₃)₂CH), 4.05 (1H, q, J=10 Hz (CH₃)₂CH), 6.82 (2H, d, J=5 Hz, phenyl-3H and 5H), 7.17 (1H, s, SCH), 7.34 (2H, d, J=5 Hz, phenyl-2H and 4H), 7.52 (2H, s, NH₂) and 7.87 (1H, d, J=5 Hz, NH)

### 13 g. Preparation of Compound 79

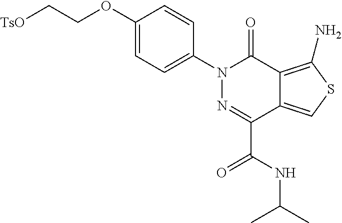 [SMILES: Cc1ccc(S(=O)(=O)OCCOc2ccc(-n3nc(C(=O)NC(C)C)c4csc(N)c4c3=O)cc2)cc1]

To 58 (0.30 g, 0.08 mmol) in anhydrous acetonitrile (20 mL), cesium carbonate (0.42 g, 1.29 mmol) and ethylene ditosylate (0.39 g, 1.02 mmol) were added. The reaction mixture was heated to 60 C for 16 h. The reaction mixture was diluted with water (25 mL) and the resulting mixture was extracted with dichlormethane (2×100 mL). The organic layer was washed with brine (20 mL), dried (Na₂SO₄), filtered, and evaporated under vacuum. The crude compound was purified using semi-prep with water and ammonium acetate as gradient solvents. The fractions were freeze-dried to yield 77 mg (16%) as yellow solid.

LC-MS: m/z calcd for C₂₅H₂₆N₅O₆S₂, 542.1, found 542.9 (M+H)⁺.

¹H NMR (500 MHz, CDCl₃): δ_{H} 1.22 (6H, d, J=5 Hz, NHCH(CH₂)₃), 2.46 (3H, s, CH₃), 4.16-4.23 (3H, m, NHCH(CH₂)₃ and SO₂OCH₂CH₂), 4.37-4.43 (2H, m, SO₂OCH₂CH₂), 6.13 (2H, s, NH2), 6.89 (2H, d, J=5 Hz, phenyl-3H and 5H), 7.28-7.49 (2H, m, tosylphenyl-3H and 5H), 7.58 (1H, s, SCH), 7.73 (2H, d, J=5 Hz, phenyl-2H and 6H) and 7.83 (2H, d, J=5 Hz, tosyl phenyl-2H and 6H).

### Example 14

### Preparation of Compound 81

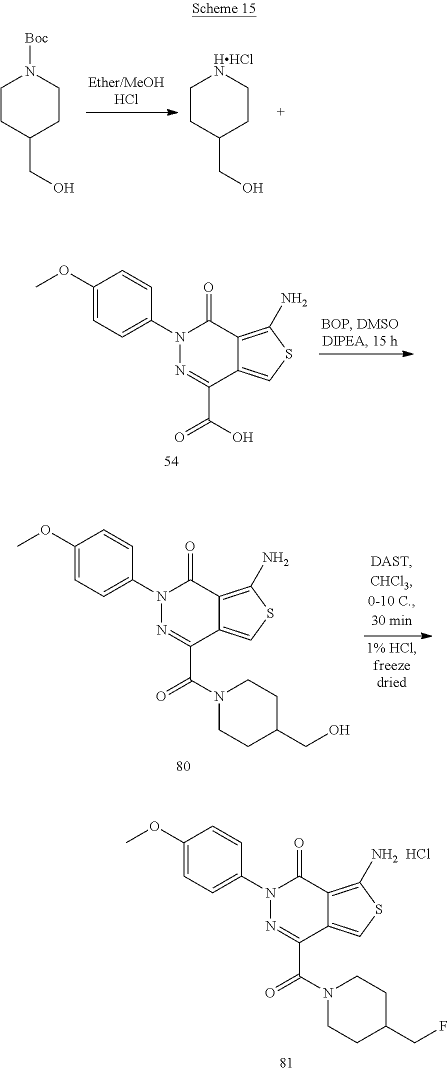

### 14a. Preparation of Compound 80

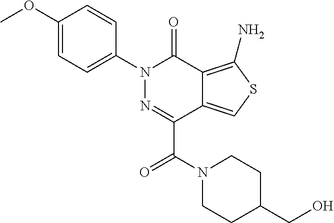 [SMILES: COc1ccc(-n2nc(C(=O)N3CCC(CO)CC3)c3csc(N)c3c2=O)cc1]

tert-Butyl 4-(hydroxymethyl)piperidine-1-carboxylate (50 mg, 0.23 mmol) was taken in a 50:50 mixture of ether and methanol (10 ml) and conc. HCl (1 mL) added to it dropwise over a period of 10 min. The reaction mixture was stirred for 1 h. The solvents were evaporated. Water was removed as an azeotrope with anhydrous acetonitrile (3×20 mL) to give the free amine as a hydrochloride salt. 54 (50 mg, 0.15 mmol) (prepared according to Example 13d) was dissolved in DMSO (2 mL) and piperidin-4-ylmethanol hydrochloride (36 mg, 0.23 mmol), DIPEA (40.7 mg, 0.31 mmol, 0.05 ml) and ((1H-benzo[d][1,2,3]triazol-1-yl)oxy)tris(dimethylamino)phosphonium hexafluorophosphate(V) (105 mg, 0.23 mmol) were added. The reaction mixture was stirred for 16 h at room temperature. The progress of the reaction was monitored by LCMS. The reaction mixture was diluted with water (50 mL) and extracted with ethyl acetate (2×15 mL). The organic layer was filtered, dried (Na₂SO₄) and concentrated to yield 35 mg (46%) of desired product as brown solid.

LC-MS: m/z calcd for C₂₀H₂₂N₄O₄S 414.1, found 415.1 (M+H)⁺.

### 14b. Preparation of Compound 81

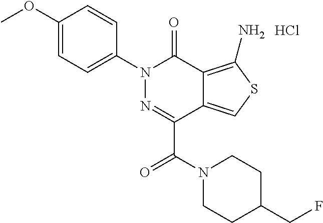 [SMILES: COc1ccc(-n2nc(C(=O)N3CCC(CF)CC3)c3csc(N)c3c2=O)cc1 ; Cl]

80 (350 mg, 0.84 mmol) was dissolved in anhydrous chloroform (20 ml) and diethylaminosulfur trifluoride (0.11 mL, 0.84 mmol) diluted with CHCl₃ (5 ml) was added at 0 C in drops over 10 min. The reaction was monitored every 10 min by TLC. Thereafter reaction mixture was washed with saturated NaHCO3 (20 mL) and extracted with ethyl acetate (2×50 mL). The organic layer was filtered, dried (Na₂SO₄) and concentrated to yield the crude product. The crude product was purified by Semi-prep HPLC using acetonitrile:methanol(50:50) and 20% ammonium acetate (pH 4.3). 1% HCl solution (5 mL) was added to the pooled fractions before freeze-drying to yield 50 mg (13%) of required compound as yellow solid.

LC-MS: m/z calcd for C₂₀H₂₁FN₄O₃S 416.1, found 417.1 (M+H)⁺.

¹H NMR (300 MHz, DMSO): δ_{H} 1.07-1.28 (2H, m, FCH₂CHCH₂CH₂), 1.61-1.83 (2H, m, FCH₂CHCH₂CH₂), 1.88-2.07 (1H, m, CH), 2.74-2.91 (2H, m, ONCH₂CH₂), 3.03-3.17 (ONCH₂CH₂), 3.79 (3H, s, OCH₃), 4.30 (2H, dd, J=3 Hz and 15 Hz, CH₂F), 6.62 (2H, s, NH₂), 6.99 (2H, d, J=3 Hz, phenyl-2H and 6H), 7.38 (2H, d, J=3 Hz, phenyl-3H and 5H) and 7.54 (1H, s, SCH).

### Example 15

### Preparation of Compound 82

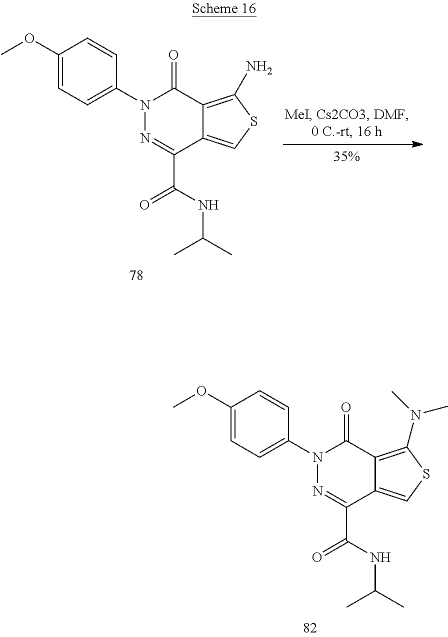

78 (50 mg, 0.14 mmol) was dissolved in 5 mL anhydrous dimethylformamide and cesium carbonate (90 mg, 0.28 mmol) added to it. The reaction mixture was maintained at 0 C and methyl iodide (39 mg, 0.28 mmol, 0.017 mL) dissolved in DMF (3 mL) and added slowly in drops over 10 min. The reaction mixture was allowed to stir at room temperature for 16 h. The reaction mixture was diluted with water (30 mL) and extracted with ethyl acetate (3×20 mL). The combined organic layers were dried (Na₂SO₄) and evaporated under vacuum. Purification was carried over neutral alumina eluting with hexane (A): ethyl acetate (B) (0-30%) (B), 8 g, 12 mL/min to give desired product 19 mg (35%) as a pale yellow solid.

LC-MS: m/z calcd for C₁₉H₂₂N₄O₃S, 386.1, found 386.9 (M+H)⁺.

¹H NMR (500 MHz, CDCl₃): δ_{H} 1.22 (6H, d, J=5 Hz, NHCH(CH₃)₂), 3.14 (6H, s, N(CH₃)₂), 3.85 (3H, s, OCH₃), 4.15-4.27 (1H, m, NHCH(CH₃)₂), 6.94-7.04 (3H, m, SCH and phenyl-3H and 5H), 7.43 (2H, d, J=5 Hz, phenyl-2H and 6H) and 8.02 (1H, s, CONH).

### Example 16

### Preparation of Compound 87

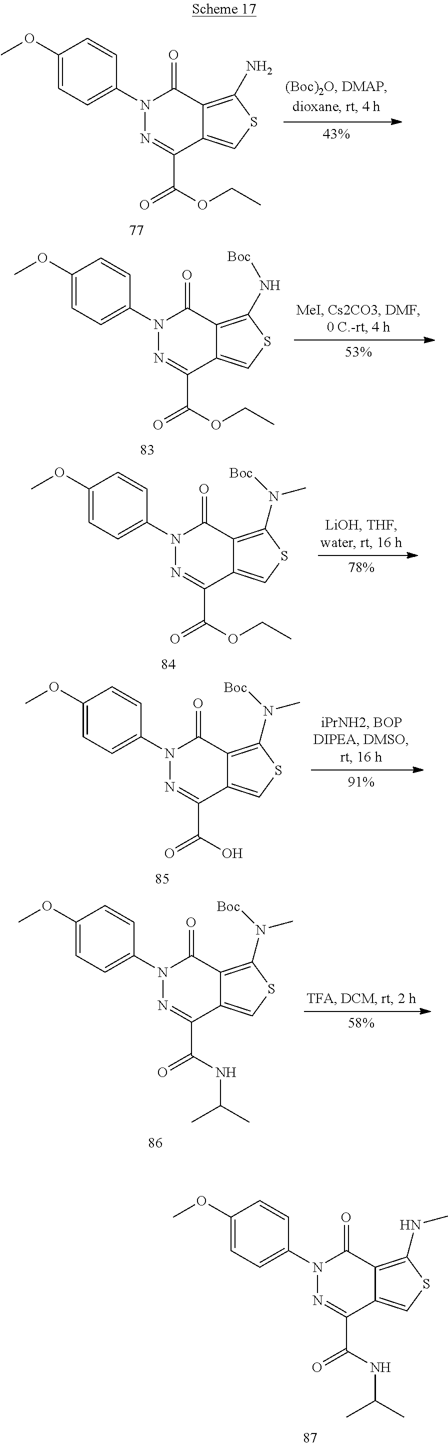

### 16a. Preparation of Compound 83

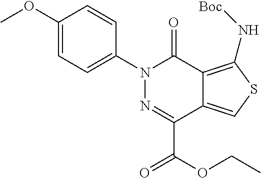 [SMILES: CCOC(=O)c1nn(-c2ccc(OC)cc2)c(=O)c2c(NC(=O)OC(C)(C)C)scc12]

77 (100 mg, 0.28 mmol) was dissolved in dioxane (7 mL) and 4-dimethylaminopyridine (0.35 mg, 0.02 mmol) was added to it. Boc anhydride (69 mg, 0.32 mmol) dissolved in dioxane (3 mL) was added dropwise to the reaction mixture at room temperature and allowed to stir for 4 h. The progress of the reaction mass was monitored by HPLC, dioxane was distilled off and the crude reaction mixture was diluted with water (15 mL) and extracted with ethyl acetate (3×15 mL). The combined organic extracts were dried (Na₂SO₄) and distilled under vacuum to obtain the crude compound. Purification was carried over neutral alumina eluting with hexane (A): ethyl acetate (B) (0-15%) (B), 8 g, 12 mL/min to give desired product 55 mg (43%) as a pale yellow solid.

LC-MS: m/z calcd for C₂₁H₂₃N₃O₆S 445.3, found 445.9 (M+H)⁺.

¹H NMR (500 MHz, CDCl₃): δ_{H} 1.43 (3H, t, J=5 Hz, CH₂CH₃), 1.53 (9H, s, O(CH₃)₃), 3.85 (3H, s, OCH₃), 4.46 (2H, q, J=10 Hz, CH₂CH₃), 6.97-7.01 (2H, m, phenyl-3H and 5H), 7.47-7.51 (2H, m, phenyl-2H and 6H), 7.78 (1H, s, SCH) and 10.17 (1H, s, NH).

### 16b. Preparation of Compound 84

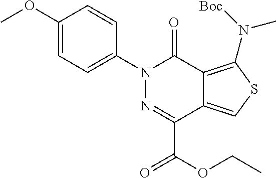

83 (55 mg, 0.12 mmol) was dissolved in 2 mL in anhydrous dimethylformamide and cesium carbonate (48 mg, 0.15 mmol) added to it. Methyl iodide (19 mg, 0.13 mmol, 0.008 mL) dissolved in 1 mL of DMF was added to the reaction mixture dropwise at 0 C. The reaction mixture was stirred at room temperature for 4 h. The reaction mixture was diluted with water (3×20 mL) and extracted with ethyl acetate (3×50 mL). The combined organic extracts were dried (Na₂SO₄) and distilled under vacuum to obtain the crude compound. Purification was carried over neutral alumina eluting with hexane (A): ethyl acetate (B) (0-25%) (B), 8 g, 12 min/min to give desired product 30 mg (53%) as a pale yellow solid.

LC-MS: m/z calcd for C₂₂H₂₅N₃O₆S 459.1, found 459.9 (M+H)⁺.

¹H NMR (500 MHz, CDCl₃): δ_{H} 1.32-1.40 (12H, m, CH₂CH₃ and O(CH₃)₃), 3.26 (3H, s, NCH₃), 3.77 (3H, s, OCH₃), 4.39 (2H, q, J=10 Hz, CH₂CH₃), 6.89-6.93 (2H, m, phenyl-3H and 5H), 7.39-7.43 (2H, m, phenyl-2H and 6H) and 8.26 (1H, s, CH).

### 16c. Preparation of Compound 85

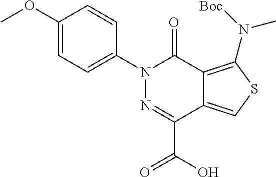

84 (30 mg, 0.06 mmol) was dissolved in a mixture of water and tetrahydrofuran (3 mL, (1:1)) and lithium hydroxide (4.7 mg, 0.19 mmol) was added to it. The reaction mixture was allowed to stir at room temperature for 16 h. The progress of the reaction was monitored by HPLC. Tetrahydrofuran was distilled off and 1N HCl was added to it till pH 6 was reached. The aqueous layer was extracted with ethyl acetate (2×50 mL). The combined organic layers were dried (Na₂SO₄) and distilled to obtain the desired 22 mg (78%) product as a pale yellow solid.

LC-MS: m/z calcd for C₂₀H₂₁N₃O₆S 431.1, found 431.9 (M+H)⁺.

¹H NMR (500 MHz, CDCl₃): δ_{H} 1.44 (9H, s, O(CH₃)₃), 3.33 (3H, s, NCH₃), 3.83 (3H, s, OCH₃), 6.96 (2H, d, J=5 Hz, phenyl-3H and 5H), 7.41 (2H, d, J=5 Hz, phenyl-2H and 6H) and 8.49 (1H, s, SCH).

### 16d. Preparation of Compound 86

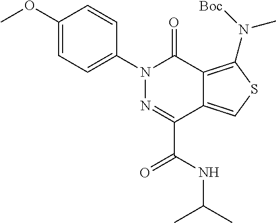

85 (22 mg, 0.05 mmol), iso-propylamine (4.5 mg, 0.07 mmol, 0.006 mL), and benzotriazole-1-yl-oxy-tris-(dimethylamino)-phosphonium hexafluorophosphate reagent (45 mg, 0.10 mmol) were suspended in anhydrous DMSO (2 mL) and DIPEA (13 mg, 0.10 mmol, 0.017 mL) was added to it. The reaction mixture was stirred at room temperature for 16 h. The progress of the reaction was monitored by LCMS. The reaction mixture diluted with water (10 mL) and the resulting mixture was extracted with ethyl acetate (2×50 mL). The organic layer was washed with brine (10 mL), dried (Na₂SO₄), filtered, and evaporated under vacuum. The residue was stirred with diethyl ether overnight. The crude compound 19 mg (91%) was taken directly for the next reaction.

LC-MS: m/z calcd for C₂₃H₂₈N₄O₅S 472.1, found 472.9 (M+H)⁺.

¹H NMR (500 MHz, CDCl₃): δ_{H} 1.24 (9H, s, O(CH₃)₃), 1.43-1.49 (6H, m, NHCH(CH₃)₂), 3.31 (3H, s, NCH₃), 3.85 (3H, s, OCH₃), 4.18-4.26 (1H, m, NHCH(CH₃)₂), 6.99-7.02 (2H, m, phenyl-3H and 5H), 7.42-7.45 (2H, m, phenyl-2H and 6H) and 8.68 (1H, s, SCH).

### 16e. Preparation of Compound 87

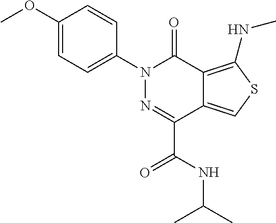

86 (19 mg, 0.04 mmol) was dissolved in 10 mL anhydrous dichloromethane and 1 mL trifluoroacetic acid added to it. The reaction was allowed to stir at room temperature for 4 h. The reaction mixture was quenched with water (10 mL) and extracted with dichloromethane (5 mL). The aqueous layer was neutralized with saturated sodium bicarbonate solution and extracted with dichloromethane (2×45 mL). The combined organic layers were dried (Na₂SO₄) and distilled to obtain 12 mg of crude compound. This was re-crystallized using ethyl acetate and hexane to give 9 mg (58%) of desired product.

LC-MS: m/z calcd for C₁₈H₂₀N₄O₃S 372.1, found 372.9 (M+H)⁺.

¹H NMR (500 MHz, CDCl₃): δ_{H} 1.22 (6H, d, J=5 Hz, NHCH(CH₂)₃), 3.04 (3H, d, J=5 Hz, NHCH₃), 3.86 (3H, s, OCH₃), 4.14-4.27 (1H, m, NHCH(CH₂)₃), 6.93 (1H, s, SCH), 6.99 (2H, d, J=5 Hz, phenyl-3H and 5H), 7.43 (2H, d, J=5 Hz, phenyl-2H and 6H) and 7.52 (1H, s, CONH).

### Example 17

### Preparation of Compound 91

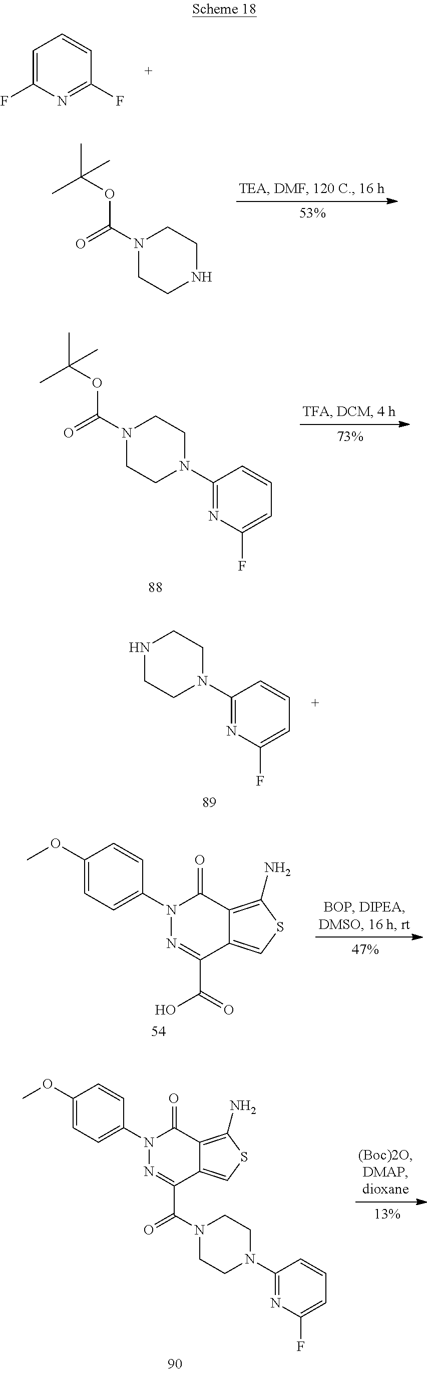 [SMILES: Fc1cccc(F)n1 ; CC(C)(C)OC(=O)N1CCNCC1 ; CC(C)(C)OC(=O)N1CCN(c2cccc(F)n2)CC1 ; Fc1cccc(N2CCNCC2)n1 ; COc1ccc(-n2nc(C(=O)O)c3csc(N)c3c2=O)cc1 ; COc1ccc(-n2nc(C(=O)N3CCN(c4cccc(F)n4)CC3)c3csc(N)c3c2=O)cc1]

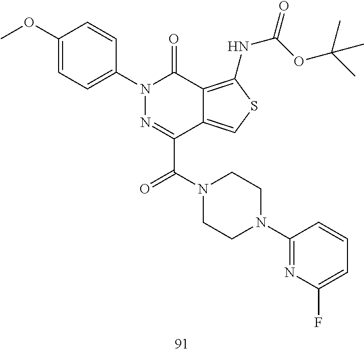 [SMILES: COc1ccc(-n2nc(C(=O)N3CCN(c4cccc(F)n4)CC3)c3csc(NC(=O)OC(C)(C)C)c3c2=O)cc1]

### 17a. Preparation of Compound 88

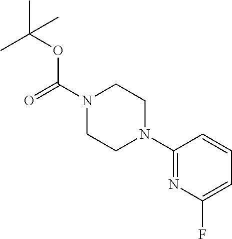 [SMILES: CC(C)(C)OC(=O)N1CCN(c2cccc(F)n2)CC1]

1-Boc-piperazine (1 g, 5.37 mmol) and 2,6-difluoropyridine (0.61 g, 5.37 mmol) were dissolved in dry DMF (20 mL) and triethylamine (0.81 g, 8.05 mmol, 1.12 mL) was added. The mixture was heated at reflux for 16 h. On cooling, the reaction was quenched with saturated sodium bicarbonate solution (15 mL). After 10 min this was diluted with water (60 mL) and the mixture extracted with ethyl acetate (3×60 mL). The combined organic layer were washed with water (2×50 mL), brine (50 mL), dried (Na2SO4), filtered and evaporated. The dark oil was put under high vacuum overnight to remove residual DMF prior to column chromatography on silica gel eluting with hexane (A): EtOAc (B) (0-15% (B), 12 g, 12 mL/min) to give the desired product 0.8 g (53%) as a viscous yellow oil.

LC-MS: m/z calcd for C₁₄H₂₀FN₃O₂, 281.2; found, 282.1 (M+H)⁺.

### 17b. Preparation of Compound 89

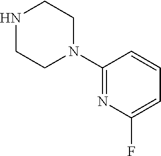 [SMILES: Fc1cccc(N2CCNCC2)n1]

88 (700 mg, 2.49 mmol) was dissolved in anhydrous dichloromethane (30 mL) and trifluoroacetic acid (10 mL) added to it. The reaction mixture was allowed to stir at room temperature for 4 h. The reaction was quenched with water, neutralized with saturated sodium bicarbonate solution. The aqueous layer was extracted with DCM (2×50 mL). The combined organic layers were dried (Na₂SO₄), filtered and evaporated to obtain the desired product 330 mg (73%) as a yellow oil.

LC-MS: m/z calcd for C₉H₁₂FN₃, 181.1; found, 181.9 (M+H)⁺.

### 17c. Preparation of Compound 90

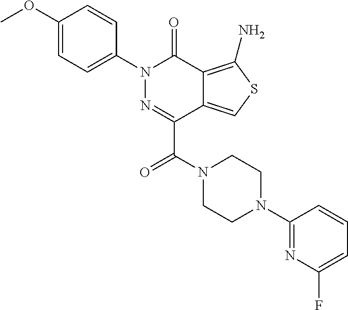 [SMILES: COc1ccc(-n2nc(C(=O)N3CCN(c4cccc(F)n4)CC3)c3csc(N)c3c2=O)cc1]

To a mixture of 54 (0.39 g, 1.23 mmol) (prepared according to Example 13d), 89 (0.33 g, 1.84 mmol), and benzotriazole-1-yl-oxy-tris-(dimethylamino)-phosphonium hexafluorophosphate reagent (0.81 g, 1.84 mmol) in anhydrous DMSO (10 mL), DIPEA (0.32 g, 2.45 mmol, 0.43 mL). was added and the reaction mixture was stirred at room temperature for 16 h. The progress of the reaction was monitored by LCMS. The reaction mixture diluted with water (25 mL) and the resulting mixture was extracted with dichloromethane (2×50 mL). The organic layer was washed with brine (20 mL), dried (Na₂SO₄), filtered and evaporated under vacuum. Purification was carried over neutral alumina eluting with hexane (A): ethyl acetate (B) (0-40%) (B), 8 g, 12 mL/min, to give desired product 0.29 g (47%) as yellow solid.

LC-MS: m/z calcd for C₂₃H₂₁FN₆O₃S, 480.1, found 480.9 (M+H)⁺.

¹H NMR (500 MHz, DMSO): δ_{H} 3.48-3.77 (8H, N(CH₂)₂(CH₂)₂N), 3.80 (3H, s, OCH₃), 6.31 (1H, s, SCH), 6.72 (2H, s, NH₂), 7.00 (2H, d, J=5 Hz, fluoropyridyl-3H and 5H), 7.42 (2H, d, J=5 Hz, phenyl-3H and 5H), 7.56 (2H, s, phenyl-2H and 6H) and 7.70 (1H, d, J=5 Hz, fluoropyridyl-4H).

### 17d. Preparation of Compound 91

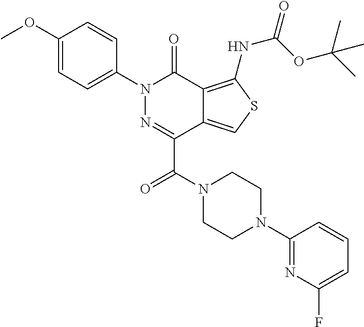 [SMILES: COc1ccc(-n2nc(C(=O)N3CCN(c4cccc(F)n4)CC3)c3csc(NC(=O)OC(C)(C)C)c3c2=O)cc1]

90 (60 mg, 0.12 mmol) was dissolved in dioxane (5 mL) and 4-dimethylaminopyridine (1.2 mg, 0.012 mmol) was added to it. Boc anhydride (30 mg, 0.13 mmol) dissolved in dioxane (3 mL) was added dropwise to the reaction mixture and heated at 50 C for 2 h. Dioxane was distilled off and the crude reaction mixture was dissolved in dichloromethane (50 mL) and washed with water (15 mL), brine (5 mL). The organic layer was dried (Na₂SO₄) and evaporated under vacuum to obtain the crude compound. Purification was carried over neutral alumina eluting with hexane (A): ethyl acetate (B) (0-20%) (B), 8 g, 12 mL/min to give desired product 10 mg (13%) as a pale yellow solid.

LC-MS: m/z calcd for C₂₈H₂₉ FN₆O₅S 580.1, found 580.9 (M+H)⁺.

¹H NMR (300 MHz, CDCl3): δ_{H} 1.54 (9H, s, O(CH₃)₃), 3.58-3.69 (4H, m, ON(CH₂)₂(CH₂)₂N), 3.77-3.82 (2H, m, ON(CH₂)₂CH₂CH₂N), 3.86 (3H, s, OCH₃), 3.89-3.95 (2H, m, ON(CH₂)₂CH₂CH₂N), 6.22 (1H, dd, J=3 Hz and 6 Hz, fluoropyridyl-3H), 6.42 (1H, dd, J=3 Hz and 6 Hz, fluoropyridyl-5H), 6.94 (2H, d, J=3 Hz, phenyl-3H and 5H), 7.31 (1H, s, SCH), 7.43 (2H, d, J=3 Hz, phenyl-2H and 6H), 7.56 (1H, q, J=6 Hz, fluoropyridyl-4H) and 10.13 (1H, s, NH).

### Example 18

### Preparation of 5-amino-N-(2-fluoroethyl)-3-(4-methoxyphenyl)-4-oxo-3,4-dihydrothieno[3,4-d]pyridazine-1-carboxamide (92)

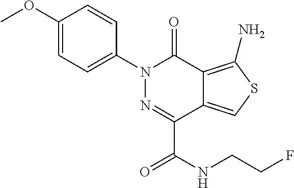 [SMILES: COc1ccc(-n2nc(C(=O)NCCF)c3csc(N)c3c2=O)cc1]

5-amino-3-(4-methoxyphenyl)-4-oxo-3,4-dihydrothieno[3,4-d]pyridazine-1-carboxylic acid (75 mg, 0.24 mmol), BOP (157 mg, 0.36 mmol) and 2-fluoroethanaminium chloride (47.1 mg, 0.47 mmol) were dissolved in anhydrous DMSO (1.5 mL) and DIPEA (0.17 ml, 0.95 mmol) added. The solution was stirred at 20° C. for 24 h. Added water (20 mL) and extracted with DCM (3×10 mL). Washed combined DCM with brine (10 mL) dried over anhydrous sodium sulfate filtered and evaporated. The residue was purified by chromatography on silica gel eluting with dichloromethane (A): methanol (B) (0.5-10% B, 10 g, 25 CV, 30 mL/min) to give the product as a yellow solid (50 mg, 58%).

LC-MS: calcd for C₁₆H₁₅FN₄O₃S, 362.1; found, 363.3 (M+H)⁺.

¹H NMR (301 MHz, CHLOROFORM-D) δ_{H} 7.54 (s, 1H, S—CH), 7.48-7.38 (m, 2H, Ar—H), 7.04-6.95 (m, 2H, Ar—H), 6.14 (br s, 2H, N—H₂), 4.56 (dt, J=48 Hz & 4.8 Hz, 2H, F—CH₂), 3.85 (s, 3H, OCH₃), 3.80-3.59 (m, 2H, NCH₂) and 3.49 (d, J=5.1 Hz, NH). ¹³C NMR (76 MHz, CHLOROFORM-D) δ_{C} 163.2 (C—NH2), 161.1 (NHC═O), 159.8 (C—OMe), 159.1 (C═O), 134.4, 133.5, 127.4 (2×Ar—CH), 126.8, 114.2 (2×Ar—CH), 107.4, 106.0 (SCH), 82.6 (d, J=168.1 Hz, C—F), 55.7 (OCH₃) and 39.8 (d, J=20.3 Hz, NHCH₂).

### Example 19

### Preparation of (R)-7-amino-4-(3-fluoropyrrolidine-1-carbonyl)-2-(4-methoxyphenyl)thieno[3,4-d]pyridazin-1(2H)-one (93)

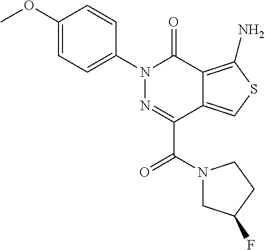 [SMILES: COc1ccc(-n2nc(C(=O)N3CC[C@@H](F)C3)c3csc(N)c3c2=O)cc1]

5-amino-3-(4-methoxyphenyl)-4-oxo-3,4-dihydrothieno[3,4-d]pyridazine-1-carboxylic acid (75 mg, 0.24 mmol), BOP (157 mg, 0.36 mmol) and (R)-3-fluoropyrrolidin-1-ium chloride (29.7 mg, 0.24 mmol) were dissolved in anhydrous DMSO (1.5 ml) and DIPEA (0.17 ml, 0.95 mmol) added. The solution was stirred at 20° C. for 24 h. Added water (20 mL) and extracted with DCM (3×10 mL). Washed combined DCM extracts with brine (10 mL) dried over anhydrous sodium sulfate filtered and evaporated. The residue was purified by chromatography on silica gel eluting with dichloromethane (A): methanol (B) (0.5-10% B, 25 g, 25 CV, 40 mL/min) to give the product as a yellow solid (60 mg, 65%).

LC-MS: calcd for C₁₈H₁₇FN₄O₃S, 388.1; found, 389.2 (M+H)⁺.

### Example 20

### Preparation of (S)-7-amino-4-(3-fluoropyrrolidine-1-carbonyl)-2-(4-methoxyphenyl)thieno[3,4-d]pyridazin-1(2H)-one (94)

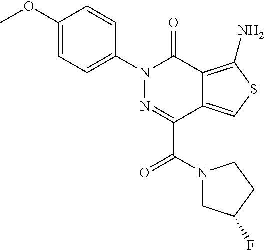 [SMILES: COc1ccc(-n2nc(C(=O)N3CC[C@H](F)C3)c3csc(N)c3c2=O)cc1]

5-amino-3-(4-methoxyphenyl)-4-oxo-3,4-dihydrothieno[3,4-d]pyridazine-1-carboxylic acid (75 mg, 0.24 mmol), BOP (157 mg, 0.36 mmol) and (S)-3-fluoropyrrolidin-1-ium chloride (29.7 mg, 0.24 mmol) were dissolved in anhydrous DMSO (1.5 ml) and DIPEA (0.17 ml, 0.95 mmol) added. The solution was stirred at 20° C. for 24 h. Added water (20 mL) and extracted with DCM (3×10 mL). Washed combined DCM with brine (10 mL) dried over anhydrous sodium sulfate filtered and evaporated. The residue was purified by chromatography on silica gel eluting with dichloromethane (A): methanol (B) (0.5-10% B, 25 g, 25 CV, 40 mL/min) to give the product as a yellow solid (50 mg, 55%).

LC-MS: calcd for C₁₈H₁₇FN₄O₃S, 388.1; found, 389.2 (M+H)⁺.

### Example 21

### Preparation of Compound 99

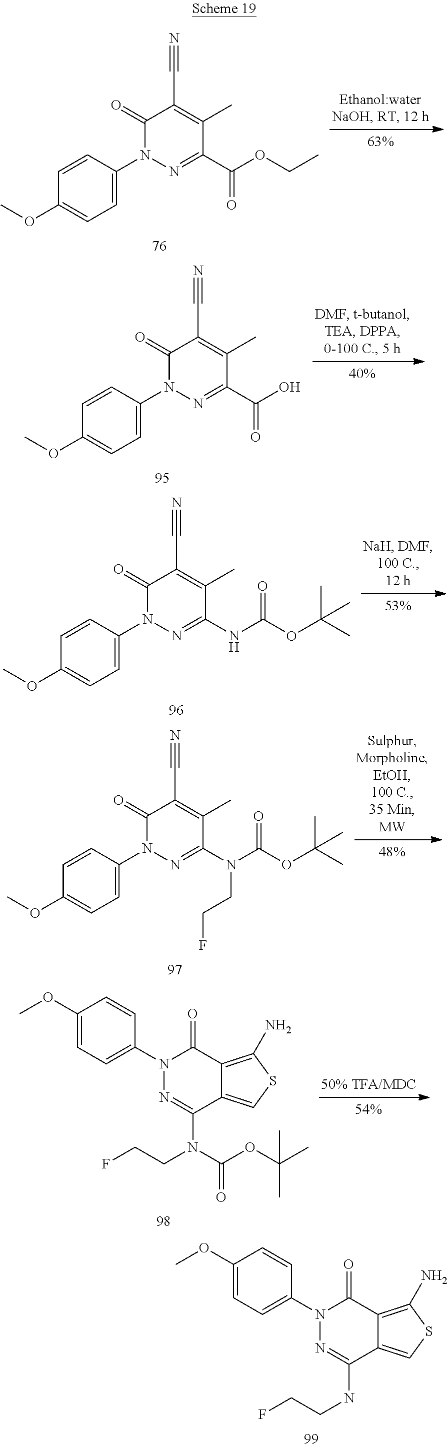

### 21a. Preparation of Compound 95

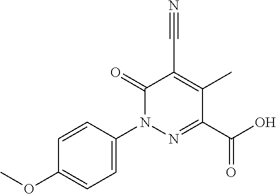 [SMILES: COc1ccc(-n2nc(C(=O)O)c(C)c(C#N)c2=O)cc1]

76 (4 g, 12.7 mmol) (prepared according to Example 13b) was suspended in a mixture of ethanol (66 mL) and water (25 mL). 0.511 g (12.7 mmol) of the sodium hydroxide was added to the reaction mass. The reaction was stirred at room temperature for 16 h. The reaction mass was concentrated under vacuum to remove the ethanol. The residue was dissolved in water (100 mL) and washed with ethyl acetate (100 mL) to remove the impurities. The pH of the aqueous reaction mass was adjusted the pH 2 by adding 1N HCl. The precipitate obtained was filtered and kept under the oven at 60 C to give 2.4 g (63%) of the desired product.

LC-MS: m/z calcd for C₁₄H₁₁N₃O₄, 285.1; found, 285.8 (M+H)⁺

¹H NMR (500 MHz, DMSOD₆): δ_{H} 2.65 (3H, s, CH₃CCCN), 3.83 (3H, s, O—CH₃), 7.09 (2H, d, J=5 Hz, Ar-3-CH and Ar-5-CH), 7.51 (2H, d, J=10 Hz, Ar-2-CH and Ar-6-CH)

### 21b. Preparation of Compound 96

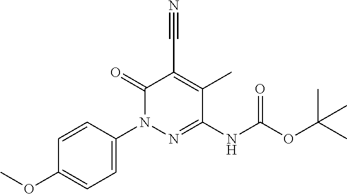 [SMILES: COc1ccc(-n2nc(NC(=O)OC(C)(C)C)c(C)c(C#N)c2=O)cc1]

95 (2 g, 7.01 mmol) was suspended in a mixture of t-butanol:DMF (40 mL, (1:1)). To this reaction mass, triethylamine (1.06 g, 10.52 mmol, 1.458 mL) was added. The reaction mass was cooled and added triphenylphosphoryl azide (2.31 g, 8.41 mmol). The reaction mass was stirred at a 0 C from another 10 min and started heating at 100 C for another 5 h. The reaction mass was quenched with water (30 mL) and extracted with ethyl acetate (5×30 mL). The organic layer was washed with water (3×20 mL) and dried over anhydrous Na₂SO₄ (15 g).The organic layer was evaporated and purified through chromatography on alumina column eluting with hexane (A): ethyl acetate (B), (0-40% (B), 8 g, 12 mL/min) to give the pure product 1.0 g (40%) as yellow solid.

LC-MS: m/z calcd for C₁₈H₂₀N₄O₄, 356.1; found, 357.15 (M+H)⁺

¹H NMR (500 MHz, CDCl₃): δ_{H} 1.53 (9H, s, OC(CH₃)₃), 2.55 (3H, s, CNCCCH₃), 3.87 (3H, s, O—CH₃), 6.60 (1H, bs, NHCOOC(CH₃)₃), 6.98 (2H, d, J=10 Hz, Ar-3-CH and Ar-5-CH) and7.53 (2H, d, J=10 Hz, Ar-2-CH and Ar-6-CH)

### 21c. Preparation of Compound 97

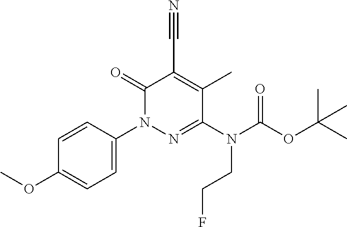 [SMILES: COc1ccc(-n2nc(N(CCF)C(=O)OC(C)(C)C)c(C)c(C#N)c2=O)cc1]

96 (0.50 g, 1.4 mmol) was taken in dry di methyl formamide (10 mL), added sodium hydride (0.04 g, 1.54 mmol) followed by the addition of fluoro ethyl tosylate (0.46 g, 2.11 mmol). The reaction mass was heated to 95 C for 12 h. Thereafter the reaction mass was quenched with water (10 mL) and extracted with ethyl acetate (4×20 mL).The organic layer was washed with water (3×20 mL) and dried over anhydrous Na₂SO₄ (15 g) and evaporated under vacuum. The crude material was purified by chromatography on alumina column eluting with hexane (A): ethyl acetate (B), (0-50% (B), 8 g, 12 mL/min) to give the pure product 0.30 g (53%) as brown liquid.

LC-MS: m/z calcd for C₂₀H₂₃FN₄O₄, 402.1; found, 402.9 (M+H)⁺

¹H NMR (500 MHz, CDCl₃): δ_{H} 1.49 (9H, bs, OC(CH₃)₃), 2.50 (3H, s, CNCCCH₃), 3.89 (3H, s, O—CH₃), 3.95-4.25 (2H, bs, NCH₂CH₂F), 4.46 (2H, m, NCH₂CH₂F),7.01 (2H, m, Ar-3-CH and Ar-5-CH) and 7.55 (2H, m, Ar-2-CH and Ar-6-CH)

### 21d. Preparation of Compound 98

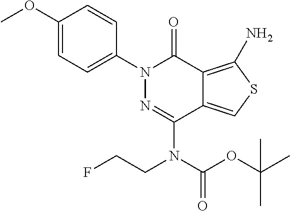 [SMILES: COc1ccc(-n2nc(N(CCF)C(=O)OC(C)(C)C)c3csc(N)c3c2=O)cc1]

97 (0.29 g, 0.716 mmol) was suspended in ethanol (5 mL), sulphur (0.03 g, 1.07 mmol) and morpholine (0.14 g, 1.43 mmol, 0.14 mL) was added it. The reaction mass was then heated at 100 C in microwave for 35 min. The ethanol was evaporated from the reaction mass and then partitioned between ethyl acetate (3×10 mL) and water (3×10 mL). The combined organic layer then dried over anhydrous Na₂SO₄ (10 g) and evaporated to dryness. The crude material the purified by chromatography on alumina column eluting with hexane (A): ethyl acetate (B), (0-60% (B), 8 g, 12 mL/min) to give the pure product 0.15 g (48%) as brownish liquid.

LC-MS: m/z calcd for C₂₀H₂₃FN₄O₄S, 434.14; found, 434.9 (M+H)⁺

¹H NMR (500 MHz, CDCl₃): δ_{H} 1.45 (9H, s, OC(CH₃)₃), 3.86 (3H, s, O—CH₃), 3.98 (1H, t, J=5 Hz, NCHₐH_{b}CH₂F), 4.03 (1H, t, J=5 Hz, NCHₐH_{b}CH₂F), 4.59 (1H, t, J=5 Hz, NCH₂CHₐH_{b}F), 4.68 (1H, t, J=5 Hz, NCH₂CHₐH_{b}F), 6.14 (2H, bs, SCNH₂), 6.44 (1H, s, CCHS), 6.99 (2H, m, Ar-3-CH and Ar-5-CH) and 7.48 (2H, m, Ar-2-CH and Ar-6-CH).

### 21e. Preparation of Compound 99

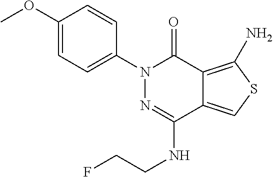 [SMILES: COc1ccc(-n2nc(NCCF)c3csc(N)c3c2=O)cc1]

98 (0.150 g, 0.345 mmol), was dissolved in dry dichloromethane (1.5 mL), cooled to 0 C using ice- salt mixture. To the reaction mass, trifluoroacetic acid: dichloromethane (5 mL, 1:1) was added. The reaction mass was stirred at RT for 12 h. Quenched with water (5 mL) and basified with saturated solution of NaHCO₃ (5 mL). The organic layer was extracted with DCM (4×5 mL) and dried over anhydrous Na₂SO₄ (5 g) and evaporated to dryness. The crude material then purified by chromatography on alumina column eluting with hexane (A): ethyl acetate (B), (0-60% (B), 8 g, 12 mL/min) to give the pure product 0.06 g (54%) as off-white solid.

LC-MS: m/z calcd for C₁₅H₁₅FN₄O₂S, 334.1; found, 334.8 (M+H)⁺

¹H NMR (500 MHz, CDCl₃): δ_{H} 3.66 (1H, m, NCHₐH_{b}CH₂F), 3.72 (1H, m, NCHₐ H_{b}CH₂F), 3.86 (3H, s, OCH₃), 4.42 (1H, bs, FCH₂CH₂NH), 4.62 (1H, t, J=5 Hz, NCH₂CHₐH_{b}F), 4.72 (1H, t, J=5 Hz NCH₂CHₐH_{b}F), 6.20 (2H, bs, SCNH₂), 6.37 (1H, s, CCHS), 6.97 (2H, d, J=10 Hz, Ar-3-CH and Ar-5-CH) and 7.55 (2H, d, J=10 Hz, Ar-2-CH and Ar-6-CH).

### Example 22

### Preparation of Compound 100

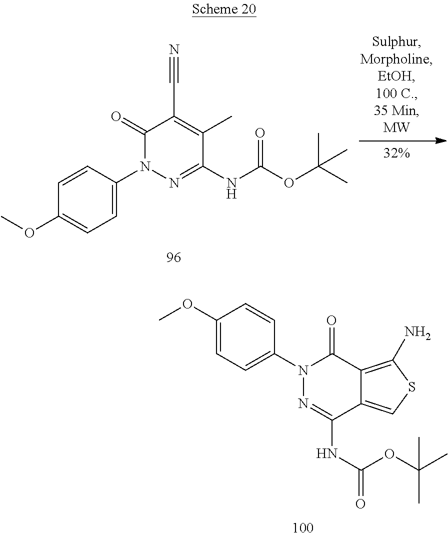

96 (0.2 g, 0.561 mmol) was taken in ethanol (2 mL), added sulfur (0.027 g, 0.84 mmol) and morpholine (0.098 g, 1.12 mmol, 0.098 mL). The reaction mass was then heated at 100 C in microwave for 35 min. The ethanol was evaporated from the reaction mass and then partitioned between ethyl acetate (3×15 mL) and water (3×15 mL). The combined organic layer then dried over anhydrous Na₂SO₄ (10 g) and evaporated to dryness. The crude material the purified by chromatography on alumina column eluting with hexane (A): ethyl acetate (B), (0-60% (B), 8 g, 12 mL/min) to give the pure product 0.07 g (32%) as brown solid.

LC-MS: m/z calcd for C₁₈H₂₀N₄O₄S, 388.1; found, 388.9 (M+H)⁺

¹H NMR (500 MHz, CDCl₃): δ_{H} 1.53 (9H, s, OC(CH₃)₃), 3.85 (3H, s, OCH₃), 6.65 (1H, bs, NHCOOC(CH₃)₃), 6.76 (1H, s, CCHS), 6.98 (2H, d, J=10 Hz, Ar-3-CH and Ar-5-CH) and 7.48 (2H, d, J=10 Hz, Ar-2-CH and Ar-6-CH).

### Example 23

### Preparation of Compound 104

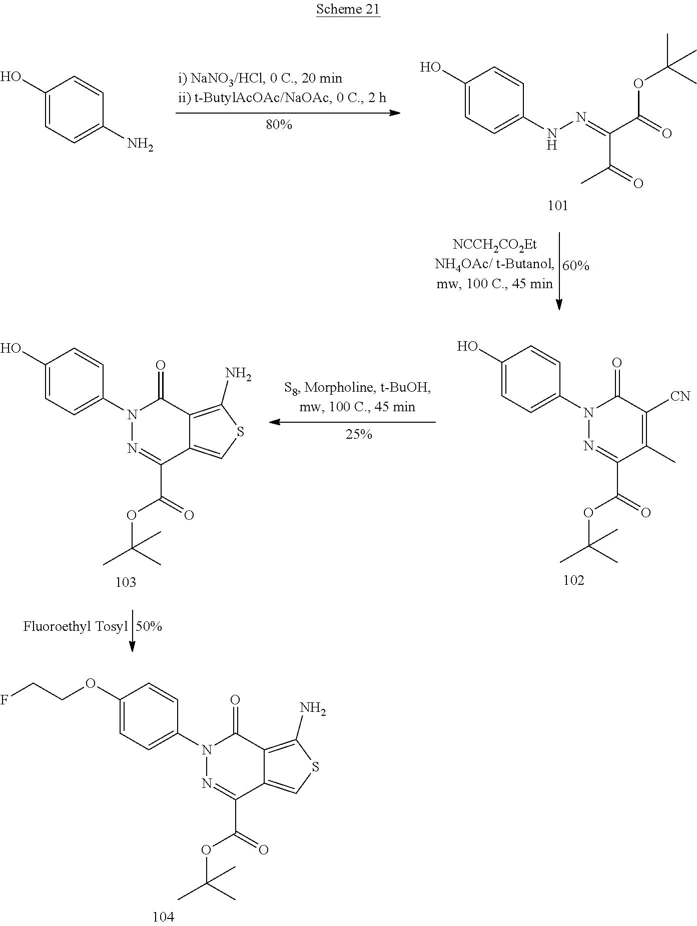 [SMILES: Nc1ccc(O)cc1 ; CC(=O)/C(=N\Nc1ccc(O)cc1)C(=O)OC(C)(C)C ; CC(C)(C)OC(=O)c1nn(-c2ccc(O)cc2)c(=O)c2c(N)scc12 ; Cc1c(C(=O)OC(C)(C)C)nn(-c2ccc(O)cc2)c(=O)c1C#N ; CC(C)(C)OC(=O)c1nn(-c2ccc(OCCF)cc2)c(=O)c2c(N)scc12]

### 23a. Preparation of Compound 101

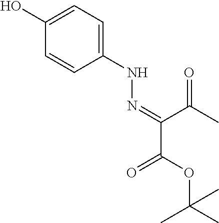 [SMILES: CC(=O)/C(=N\Nc1ccc(O)cc1)C(=O)OC(C)(C)C]

p-Anisidine (2 g, 16.23 mmol) was dissolved in a mixture of 37% hydrochloric acid (3 mL), ethanol (5 mL) and water (3 mL). The reaction mixture was cooled to 0 C, and a solution of sodium nitrite (1.12 g, 16.23 mmol) in water (7 mL) was added dropwise. The resulting mixture was stirred at 0 C for 20 min. Sodium acetate (8.61 g, 63.27 mmol) in water (20 mL) and t-Butyl acetoacetate (2.56 g, 16.23 mmol, 2 mL) were added and the reaction mixture was stirred at 0 C for 2 h. The precipitate formed was filtered, washed with water, and dried under high vacuum to give 4.08 g (80%) as brown solid.

LC-MS: m/z calcd for C₁₃H₁₆N₂O₄ 278.2, found 277 (M).

¹H NMR (500 MHz, CDCl3): δ 1.6 (5H, s, C(CH3)3), 1.62 (4H, s, C(CH3)3), 2.48 (1.67H, s, COCH3), 2.56 (1.27H, s, COCH3), 6.9 (2H, m, phenyl-CH), 7.26 (1H, d, j=10 hz, phenyl-CH) and 7.33 (1H, d, J=10 hz, phenyl-CH).

### 23b. Preparation of Compound 102

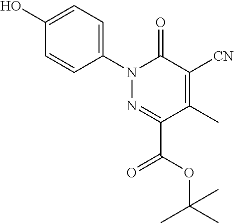 [SMILES: Cc1c(C(=O)OC(C)(C)C)nn(-c2ccc(O)cc2)c(=O)c1C#N]

A mixture of 101 (4.0 g, 14.39 mmol), ethyl cyanoacetate (3.25 g, 28.78 mmol, 1.61 mL) and ammonium acetate (4.43 g, 57.56 mmol) in t-Butanol (5 mL) was irradiated in microwave at 100 C for 45 min. The resulting mixture was distilled to remove t-Butanol and then diluted with water (100 mL) and extracted with ethyl acetate (3×100 mL). The combined organic extract was washed with water (30 mL), brine (30 mL), dried over sodium sulfate and evaporated under vacuum. The crude compound was heated in ethanol and filtered hot to yield 2.8 g (60%) as a dark yellow solid.

LC-MS: m/z calcd for C₁₆H₁₅N₃O₄ 327.12, found 328.1 (M+H)⁺

¹H NMR (500 MHz, CDCl3): δ 1.62 (9H, s, C(CH3)3), 2.72 (3H, s, CNCCCH3), 6.9 (2H, d, J=10 hz, ArCH), 7.4 (2H, d, J=10 Hz ArCH3)

### 23c. Preparation of Compound 103

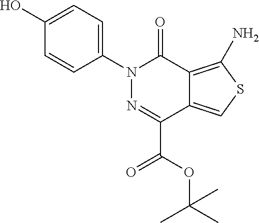 [SMILES: CC(C)(C)OC(=O)c1nn(-c2ccc(O)cc2)c(=O)c2c(N)scc12]

A mixture of 102 (2.8 g, 8.56 mmol), sulfur (0.42 g, 12.40 mmol) and morpholine (1.3 g, 17.12 mmol, 1.1 mL) in tert-Butanol (7 mL) was heated to 100 C in microwave for 30 min After the mixture was cooled, the precipitate formed was filtered and washed using ethanol to yield 0.76 g (25%) as a pale brown solid.

LC-MS: m/z calcd for C₁₆H₁₅N₃O₄S 359.09, found 360.09 (M+H)⁺.

¹H NMR (500 MHz, CDCl₃): δ 1.55 (9H, s, C(CH₃)₃), 6.64 (1H, s, ArCH), 6.8 (2H, d, J=10 Hz, ArH), 7.0 (1H, s, SCH), 7.3 (2H, d, J=10 Hz, ArH)

### 23d. Preparation of Compound 104

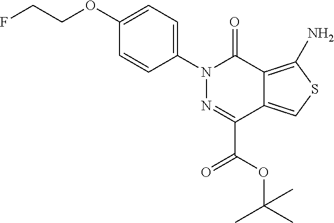 [SMILES: CC(C)(C)OC(=O)c1nn(-c2ccc(OCCF)cc2)c(=O)c2c(N)scc12]

A mixture of 103 (0.76 g, 2.12 mmol), fluoroethyltosylate (0.92 g, 4.24 mmol) and Cesium carbonate (1.22 g, 6.35 mmol) in DMF (10 mL) was stirred at ambient temperature for 16 h. The reaction was quenched in to 25 mL of water and extracted using dichlomethane (25 mL×2). The organic layer was dried using sodium sulfate and concentrated to dryness to yield 0.42 g of crude product.

LC-MS: m/z calcd for C19H20FN3O4S 405.12, found 406.12 (M+H)⁺.

1H NMR (500 MHz, CDCl3): δ 1.64 (9H, s, C(CH3)3), 4.24 (1H, t, J=5 hz, OCH2), 4.3 (1H, t, J=5 Hz,OCH2), 4.75 (1H, t, J=5 Hz, FCH2), 4.85 (1H, t, J=5 Hz, FCH2), 7.02 (2H, d, J=10 Hz, ArCH), 7.15 (1H, s SCH), 7.55 (2H, d, J=10 Hz Ar CH).

### Example 24

### Preparation of Compound 105

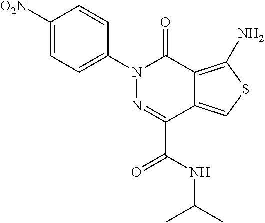 [SMILES: CC(C)NC(=O)c1nn(-c2ccc([N+](=O)[O-])cc2)c(=O)c2c(N)scc12]

Compound 105 was prepared using the same route as that shown in Example 13, starting with 4-nitroaniline rather than p-anisidine.

### Example 25

### Preparation of Compound [¹⁸F]56

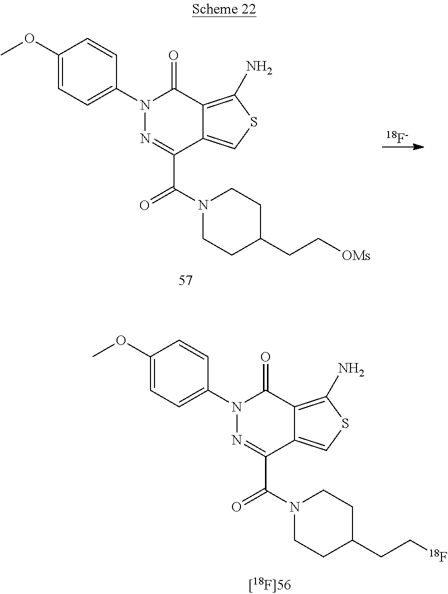

[¹⁸F]Fluoride is azeotropically dried in a Wheaton vial as described in Example 1. The vial is cooled to room temperature and a solution of mesylate 57 (2.0 mg, 3.9 mmol) in anhydrous DMSO (0.2 mL) is added. The reaction mixture is heated for 15 minutes at 100° C. The reaction product [¹⁸F]56 is isolated and formulated as described in Example 1.

### Example 26

### Preparation of Compound [¹⁸F]59

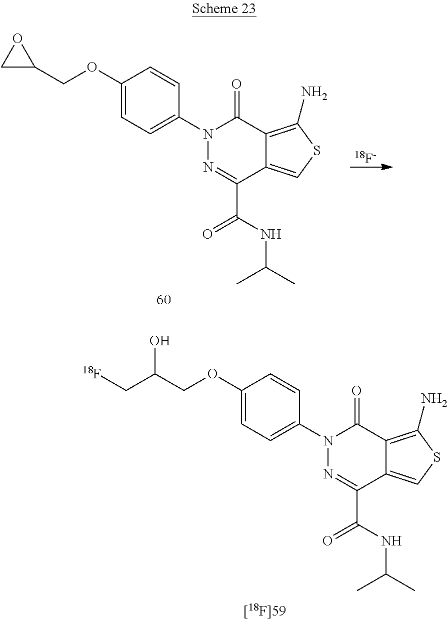

Compound [¹⁸F]59 is obtained by nucleophilic ring opening reaction of epoxide 60 using [¹⁸F]fluoride/Kryptofix in tert-amyl alcohol following a published protocol (R. Schirrmacher et al., Tetr. Lett. 52 (2011) 1973-1976).

### Example 27

### Preparation of Compound [¹⁸F]106

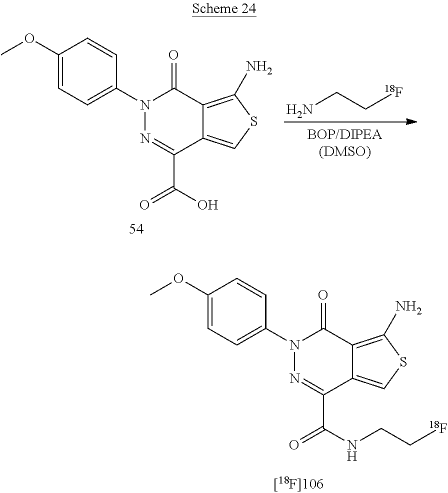

A solution of [¹⁸F]2-fluoroethylamine (100 μL acetonitrile; obtained following a method by M. Glaser et al., J. Label. Compd. Radiopharm. 2012, 55, 326-331) is added to a mixture of acid 54 (2.0 mg, 6.3 mmol), BOP (4.2 mg, 9.4 mmol), DIPEA (57 μL, 325 μmol). After incubation for 30 min at room temperature the amide [¹⁸F]106 is isolated by preparative HPLC.

### Example 28

### Preparation of Compounds [¹⁸F]67 and [¹⁸F]90

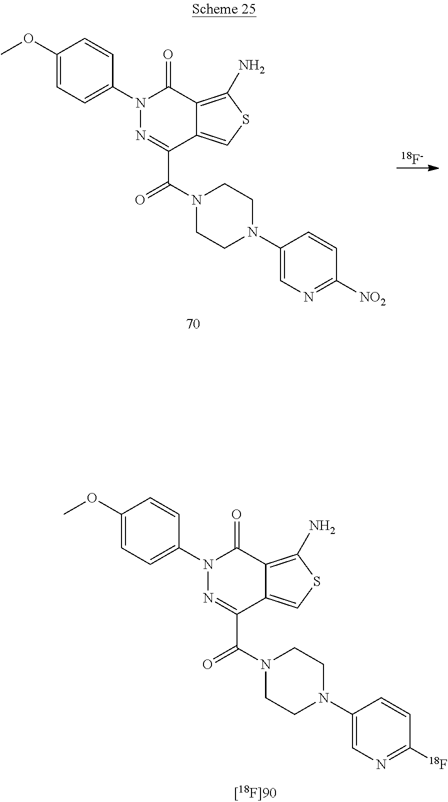

Compound [¹⁸F]90 is obtained by reacting nitro precursor 70 with the K[¹⁸F]F-K₂₂₂-carbonate complex in DMSO following the procedure as described by A. Maisonial et al. (J. Med. Chem. 54, 2011, 2745-2766). Compound [¹⁸F]67 is prepared in a similar fashion.

### Example 29

### Preparation of Compound [¹⁸F]92

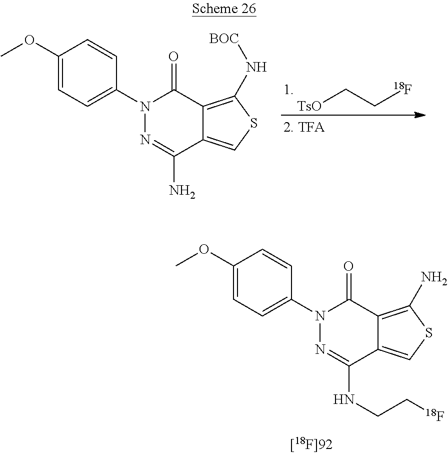 [SMILES: COc1ccc(-n2nc(N)c3csc(NC(=O)OC(C)(C)C)c3c2=O)cc1 ; "1." ; Cc1ccc(S(=O)(=O)OCC[18F])cc1 ; COc1ccc(-n2nc(NCC[18F])c3csc(N)c3c2=O)cc1]

The labelling reagent [¹⁸F]fluoroethyl tosylate is obtained following a published protocol as described by W. Wadsak et al. (Nucl. Med. Biol. 34, 2007, 1019-1028). The subsequent N-alkylation and deprotection provides [¹⁸F]92 in a similar fashion as described by E. Schirrmacher et al. (Bioorg. Med. Chem. Lett. 13, 2003, 2687-2692).

### Example 30

### Preparation of Compounds [¹⁸F]93 and [¹⁸F]94

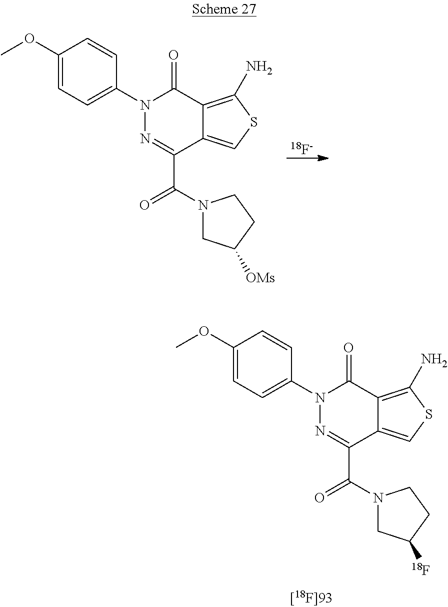

Compounds [¹⁸F]93 and [¹⁸F]93 are obtained from the corresponding mesylate precursors by reacting with the K[¹⁸F]F-K₂₂₂-carbonate complex in DMSO following the procedure as described by X.-S. He et al. (J. label. Cpd. Radiopharm. 33, 1993, 573-581).

### Example 31

### General

A number of novel compounds were screened for their ability to bind tau+ neurofibrillary tangles in Alzheimer disease brain tissue, in vitro ADME properties and brain uptake in vivo. Taken together, the results demonstrate that a selection of compounds of the invention bind preferentially to tangles, are metabolically stable in vitro, can be radiolabelled, and have a high brain uptake in rodent models. Thus, such compounds display the desired characteristics for a tau imaging agent.

### 1 Material and Methods

### 1.1 Human Tissue

Human brain tissue samples from the entorhinal cortex of patients with Alzheimer's disease (AD) and healthy controls were obtained from Tissue Solutions (Tissue Solutions Ltd, Glasgow, UK). Tissue samples were collected after informed written consent, and specimens were dissected at the tissue bank and snap-frozen for cryopreservation at a time interval of between 3 and 18 h after death. The frozen tissue was embedded in TissueTek® (VWR) and sectioned using a Microm HM560 cryostat (Thermo Scientific). Serial 12 μm sections were mounted onto SuperFrost®+ glass slides (VWR) and stored a −70° C.

### 1.2 Immunohistochemistry

To confirm presence and location of tau+ neurofibrillary tangles (NFTs) and β-amyloid (Aβ)+ plaques in the human tissue sections, every 20^{th} tissue section throughout the specimens was processed for immunohistochemical labelling with antibodies raised against aggregated and hyperphosphorylated tau, and aggregated Aβ.

Briefly, tissue sections were defrosted and fixed in ice-cold 70% ethanol. All tissue sections were rinsed with PBS after fixation and between all subsequent incubation steps. Following fixation, tissue sections were incubated first with H₂O₂ (EnVision™ kit, Dako). Tissue sections to be processed for Aβ immunohistochemistry were further treated for antigen retrieval by incubation in 70% formic acid (Sigma-Aldrich) for 10 min. All tissue sections were then incubated with 10% normal goat serum (Vector Labs) to block non-specific labelling. After the blocking steps, the tissue sections were incubated with primary antibodies raised against tau (AT8, mouse monoclonal antibody, 1:20 dilution, Invitrogen) or Aβ (4G8, mouse monoclonal antibody, 1:100 dilution, Covance) for 1 h at room temperature (RT).

Following incubation with primary antibodies, the tissue sections were incubated with secondary antibodies conjugated to horseradish peroxidase (HRP) directed against mouse IgG for 30 min at RT. This was followed by incubation with the chromogen 3,3′-diaminobenzidine (DAB) for 2-3 min. EnVision™ HRP kits were used for secondary labelling (Dako). Finally the sections were counterstained with haematoxylin (Merck), dehydrated and mounted in DPX mounting media (VWR). Images of tissue sections labelled with tau and Aβ were captured using a Nikon digital camera connected to a Leica microscope and using the NIS Elements D software (Nikon). Images were further processed with the Photoshop® software (Adobe).

### 1.3 Gallyas Silver Stain

Conventional immunohistochemistry rely on the presence and detection of specific antigen by primary antibodies. For example, the tau antibody (AT8) used for the immunohistochemical detection of NFTs in 1.2 detects a specific conformation of hyperphosphorylated tau aggregates, but it will not detect less mature tau aggregates (Augustinack et al., 2002). Likewise, further phosphorylation results in conformational changes and loss of the AT8 specific tau antigen (Augustinack et al., 2002, Jeganathan et al., 2008). It has therefore been suggested that using a different method, such as Gallyas silver stain, that doesn't rely on one antigen is a more sensitive and accurate method to detect and label NFTs (Rosenwald et al., 1993, Cullen et al., 1996, Uchihara et al., 2001, Uchihara, 2007). Therefore, in addition to tau+ and Aβ+ immunohistochemistry, tissue sections adjacent to the slides used for immunohistochemistry where processed for Gallyas silver stain.

Briefly, tissue sections were defrosted and fixed for 10 min in neutral buffered formalin (VWR) and washed first in PBS and then dH₂O. Unless stated otherwise, tissue sections were rinsed in dH₂O between each of the subsequent incubation steps. First, the tissue sections were incubated in 5% periodic acid for 5 min, and then for 1 min in an alkaline silver iodide solution. This was followed by a 10 min wash in 0.5% acetic acid, and then the tissue sections were incubated in developer solution for 5-30 min. The tissue sections were then washed in 0.5% acetic acid and rinsed in dH₂O. This was followed by incubation for 5 min in a 0.1% gold chloride solution, and then 5 min in 1% sodium thiosulphate solution. The tissue sections were then rinsed in tap water and counterstained with 0.1% nuclear fast red for 2 min.

Finally, the tissue sections were rinsed in tap water, dehydrated and mounted in DPX mounting media (VWR). All reagents for the Gallyas silver stain were procured from Sigma-Aldrich unless otherwise stated. Images of tissue sections labelled with tau and Aβ were captured using a Nikon digital camera connected to a Leica microscope and using the NIS Elements D software (Nikon). Images were further processed with the Photoshop® software (Adobe).

### 1.4 Tissue Binding Assay

The binding of compounds to tau+NFTs and Aβ+ plaques in human AD tissue were evaluated based on fluorescence. All test compounds have an innate fluorescence, and binding of the compounds to NFTs/plaques in AD tissue can therefore be detected using a fluorescence microscope. Two reference compounds were included in the screen; PiB (Pittsburgh compound B (PiB) and FDDNP (fluorescent probe 2-(1-(2-(N-(2-fluoroethy)-N-methylamino)-naphthalene-6-yl)-ethylidende)-malononitrile). PiB has been reported to bind with a preference to Aβ+ plaques (Ikonomovic et al., 2008), whereas FDDNP binds to both NFTs and plaques (Agdeppa et al., 2001). In addition, an aminothienopyrazidine compound (ATPZ), a tau aggregation inhibitor first reported by Ballatore et al (Ballatore et al., 2010), was also screened on tissue.

Briefly, tissue sections were defrosted and fixed in ice-cold 70% ethanol. All tissue sections were rinsed with PBS after fixation and between all subsequent incubation steps. To quench autofluorescence, tissue sections were incubated first with 0.25% KMnO₄ (Sigma-Aldrich) in PBS for 12 min, and then with 0.1% K₂₅₂O₅/0.1% oxalic acid (both reagents from Sigma-Aldrich) in PBS for 1 min. The tissue sections were blocked with 2% BSA in PBS for 10 min, and then incubated with the test compounds at 100 μM concentration for 1 h at RT. Compounds with positive binding at 100 μM was further tested in subsequent assays using lower test concentrations, 10 μM and 1 μM. Finally the tissue sections were rinsed in PBS, and mounted in SlowFade® mounting media (Invitrogen). Images of labelled tissue sections were captured using a Nikon digital camera connected to a Leica microscope and using the NIS Elements D software (Nikon). Images were further processed with the Photoshop® software (Adobe).

### 1.5 In Vitro ADME Screening

Test compounds were screened using a panel of in vitro ADME assays for prediction of in vivo properties. The following assays were used; parallel artificial membrane permeability assay (PAMPA) to determine cell membrane passage, compound stability in the presence of human or rat plasma, compound stability in the presence of human or rat liver microsomes, and determination of binding to proteins in human plasma and rat brain homogenates. To enable comparison with two compounds reported to have high brain uptake in vivo, PiB (Ikonomovic et al., 2008) and ATPZ (Ballatore et al., 2010) were included in the screen.

### 1.5.1 PAMPA

The PAMPA assay is used to determine how well a compound crosses a cell membrane by measuring its passage through a phosphotidyl choline barrier. A permeability coefficient >−6 indicates high permeability across lipid membranes and is indicative of a compounds ability to cross the blood brain barrier.

A 10 μM solution was incubated on a PDVF membrane coated with a 2% phoshotidyl choline solution for 5 h at RT. Membrane penetration was measured using LC-MS.

### 1.5.2 Protein Binding Assays

The protein binding assays provide an estimate of free (unbound) fraction of the compound in the blood or brain in vivo. High protein binding of a compound within the blood indicates that it is potentially unavailable for passage across the blood brain barrier and could compromise its metabolism or excretion, whereas high binding to proteins in the brain is indicative of non-specific binding and slow excretion. The desirable criterion for this assay is <99% of test compound bound.

Test compounds were first dissolved in DMSO to a concentration of 50 μM. This was followed by incubation in samples of human plasma and rat brain homogenates (final test concentration 1 μM). Binding of compounds to proteins was determined in the samples by rapid equilibrium dialysis after 5 and 30 min of incubation.

### 1.5.3 Liver Microsome Stability Assay

The liver microsome stability assay provides an estimate of compound stability and rate of metabolism in vivo. The desirable criterion for this assay is >50% parent compound after 30 min.

A 1 μM compound solution was incubated with rat or human liver microsomes (20 mg/ml) at 37° C. and the amount of parent compound remaining following the incubation was determined after 5 and 30 min of incubation using LC-MS.

### 1.6 In Vivo Cold Bio-Distribution

All animal studies were in compliance with local rules and regulations. Test compounds were administered by intravenous (i.v) injection through the tail vein of naïve male Wistar rats (50 μg test compound/rat). The animals were sacrificed by dislocation of the neck at 2, 10, 30, 60 min post-injection (p.i). The brain and plasma were collected from each animal. The concentration of test compound was measured in the plasma and brain homogenates using LS-MS, and calculated as % compound/g (% ID/g).

1.7 In Vivo Bio-Distribution with Radiolabelled Compounds

All animal studies were in compliance with local rules and regulations. [¹⁸F]-radiolabelled compounds were injected i.v through the tail vein of naïve male C57B1/6 mice (2 MBq/mouse). The animals were sacrificed by dislocation of the neck at 2, 10, 30 and 60 min p.i. Next, the animals were dissected and the radioactivity of organs, tissue and blood was measured using a Wallac γ counter (Perkin-Elmer). The compound concentration in the specimens was calculated as % ID/g.

### 2 Results

2.1 Histology of human AD tissue

Every 20^{th} section was labelled for tau or Aβ to confirm the presence and extent of tau+ NFTs and Aβ+ plaques in the human tissue sections. Adjacent tissue sections were processed using Gallyas silver stain, which is a sensitive method for labelling of NFTs and neuritic plaques that is not relying on antibodies for detection.

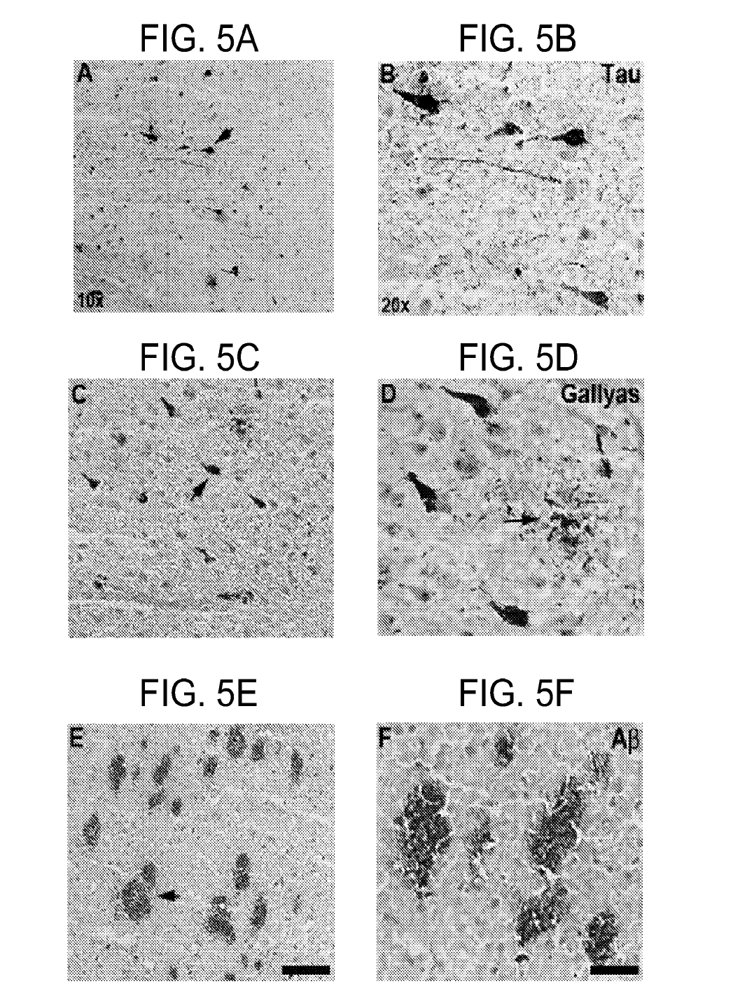

Numerous tau+ NFTs and neuritic plaques, as well as Aβ+ plaques, were observed in all AD specimens. In contrast, no NFTs or plaques were observed in tissue sections from a control subject. NFTs and neuritic plaques were also observed in tissue sections labelled with Gallyas silver stain. More mature NFTs were detected with Gallyas silver stain compared to tau+ immunohistochemistry. Typical morphology of NFTs and plaques are demonstrated FIG. 5.

### 2.2 Screening of Compound Binding to Human AD Tissue

The binding of compounds to tau+ NFTs and Aβ+ plaques in human AD tissue were evaluated based on fluorescence. All test compounds have an innate fluorescence, and binding of the compounds to NFTs/plaques in AD tissue can therefore be detected using a fluorescence microscope. The results from the tissue assays are summarized Table 2, Table 3, and Table 4.

At high test concentration, binding of both reference compounds (PiB and FDDNP) was detected to both NFTs and plaques (Table 2). At lower test concentrations, PiB only bound to plaques. These results are as expected and are supported by reports in the literature (Agdeppa et al., 2001, Ikonomovic et al., 2008, Thompson et al., 2009).

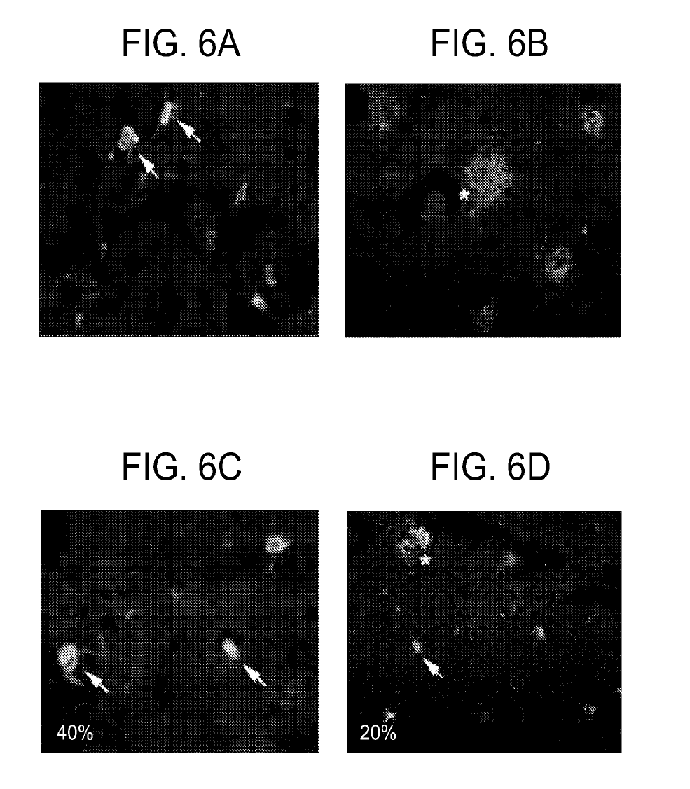

Some of the tested novel compounds were observed to bind to NFTs (Table 2, Table 3, and Table 4). Most notably are test compounds 38 and 105 (Table 1, FIG. 6 (38 A-B, 105 C-D)), which at high test concentrations bind to both NFTs and plaques but at lower test concentrations bind with a preference for NFTs.

### 2.3 In Vitro ADME Screening

Selected compounds were screened using multiple in vitro assays for prediction of in vivo properties.

The results are summarized in Table 3. The data suggest that the majority of the screened novel compounds from this class fulfil the desired in vitro criteria for an imaging agent, and these compounds are predicted to cross BBB and to be metabolically stable in vivo.

### 2.4 Cold Bio-Distribution

Selected compounds were screened using cold bio-distribution in rat to determine brain uptake. The results are summarized in Table 6. The data demonstrates uptake >0.2% ID/g at 2 min p.i. for 38 and 99, which suggest significant brain uptake, but low brain uptake of 106. In addition, the clearance ratio for 38 and 99 demonstrates rapid brain uptake followed by rapid clearance. For cold bio-distribution in rats, the benchmark criteria for an imaging agent are a brain uptake >0.2% ID/g at 2 min p.i. and a clearance ratio >2.

2.5 Biodistribution with [¹⁸F]-Radiolabelled Compounds

Selected compounds were radiolabelled and used for bio-distribution in mice to determine brain uptake. The results are summarized in Table 7. The data demonstrate uptake >1% ID/g at 2 min p.i. for [¹⁸F]-38 (i.e., 38*). In addition, the clearance ratio for [¹⁸F]-38 (i.e., 38*) demonstrates a rapid brain uptake followed by rapid clearance. For bio-distribution of radiolabelled compounds in mice, the minimum criteria required for an imaging agent are a brain uptake >1% ID/g at 2 min p.i. and a clearance ratio >2.

TABLE 2
Results from tissue binding assay.

| Compound | Structure | Concentration (μM) | NFTs | Plaques |
|---|---|---|---|---|
| PIB | 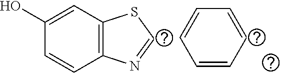 | 100 10 1 | +++ + − | +++ ++ ++ |
| FDDNP | 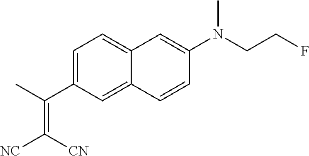 [SMILES: CC(=C(C#N)C#N)c1ccc2cc(N(C)CCF)ccc2c1] | 100 10 1 | +++ ++ + | +++ ++ + |
| ATPZ | 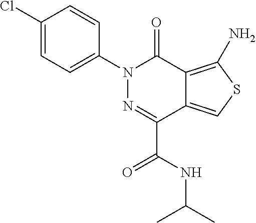 [SMILES: CC(C)NC(=O)c1nn(-c2ccc(Cl)cc2)c(=O)c2c(N)scc12] | 100 10 1 | +++ ++ + | + − − |
| 38 | 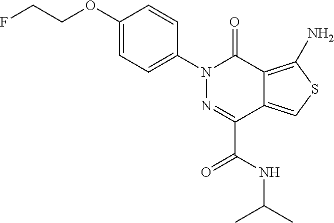 [SMILES: CC(C)NC(=O)c1nn(-c2ccc(OCCF)cc2)c(=O)c2c(N)scc12] | 100 10 1 | +++ ++ + | ++ + − |
| 14 | 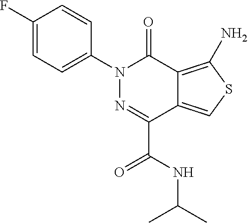 [SMILES: CC(C)NC(=O)c1nn(-c2ccc(F)cc2)c(=O)c2c(N)scc12] | 100 10 1 | + − − | + − − |
| 105 | 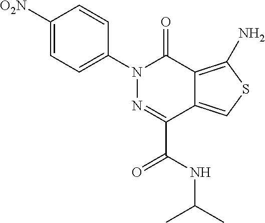 [SMILES: CC(C)NC(=O)c1nn(-c2ccc([N+](=O)[O-])cc2)c(=O)c2c(N)scc12] | 100 10 1 | ++ + − | ++ − |
| 82 | 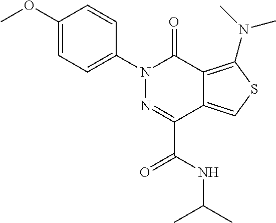 | 100 | + | ++ − − |
| 90 | 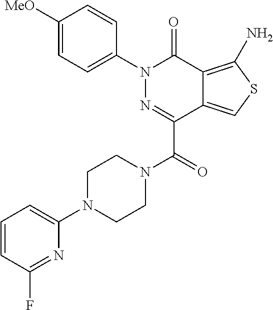 [SMILES: COc1ccc(-n2nc(C(=O)N3CCN(c4cccc(F)n4)CC3)c3csc(N)c3c2=O)cc1] | 100 | (+) | − |
| 63 | 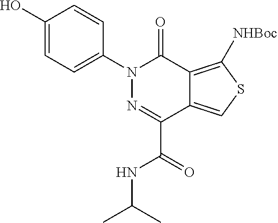 [SMILES: CC(C)NC(=O)c1nn(-c2ccc(O)cc2)c(=O)c2c(NC(=O)OC(C)(C)C)scc12] | 100 | − | (+) |
| 91 | 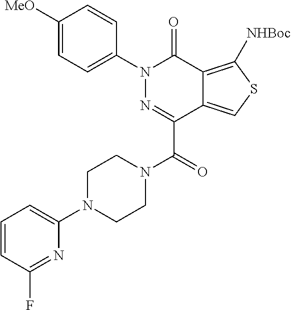 [SMILES: COc1ccc(-n2nc(C(=O)N3CCN(c4cccc(F)n4)CC3)c3csc(NC(=O)OC(C)(C)C)c3c2=O)cc1] | 100 | − | − |

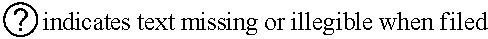

− No staining;
+ weak staining;
++ moderate staining;
+++ intense staining

TABLE 3
Results from tissue binding assay

| | | Concentration | | |
|---|---|---|---|---|
| Compound | Structure | (μM) | NFTs | Plaques |
| 64 | 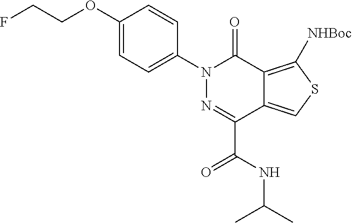 [SMILES: CC(C)NC(=O)c1nn(-c2ccc(OCCF)cc2)c(=O)c2c(NC(=O)OC(C)(C)C)scc12] | 100 | − | − |
| 56 | 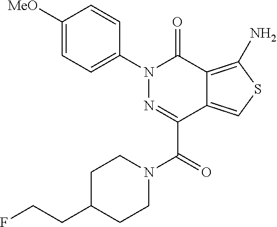 [SMILES: COc1ccc(-n2nc(C(=O)N3CCC(CCF)CC3)c3csc(N)c3c2=O)cc1] | 100 | − | − |
| 81 | 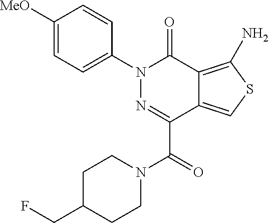 [SMILES: COc1ccc(-n2nc(C(=O)N3CCC(CF)CC3)c3csc(N)c3c2=O)cc1] | 100 | | − |
| 106 | 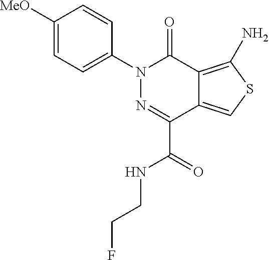 [SMILES: COc1ccc(-n2nc(C(=O)NCCF)c3csc(N)c3c2=O)cc1] | 100 10 1 | | − |
| 87 | 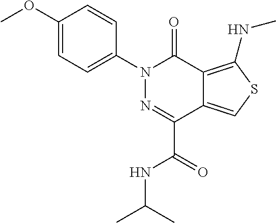 | | − | − |
| 107 | 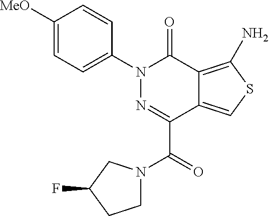 [SMILES: COc1ccc(-n2nc(C(=O)N3CC[C@@H](F)C3)c3csc(N)c3c2=O)cc1] | 100 | − | + − − |
| 108 | 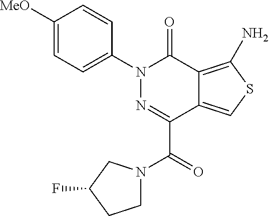 [SMILES: COc1ccc(-n2nc(C(=O)N3CC[C@H](F)C3)c3csc(N)c3c2=O)cc1] | 100 | − | − |
| 99 | 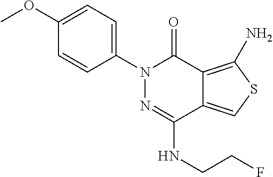 [SMILES: COc1ccc(-n2nc(NCCF)c3csc(N)c3c2=O)cc1] | 100 | ND¹ | ND¹ |
| 67 | 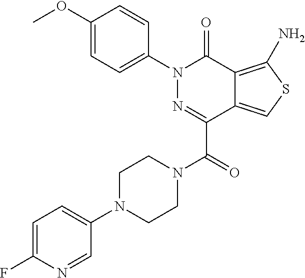 [SMILES: COc1ccc(-n2nc(C(=O)N3CCN(c4ccc(F)nc4)CC3)c3csc(N)c3c2=O)cc1] | 100 | − | − |

¹Not possible to determine binding due to test compound fluorescence in the UV range
− No staining;
+ weak staining;
++ moderate staining;
+++ intense staining

TABLE 4
Results from the tissue binding assay

| | | Concentration | | |
|---|---|---|---|---|
| Compound | Structure | (μM) | NFTs | Plaques |
| 59− | 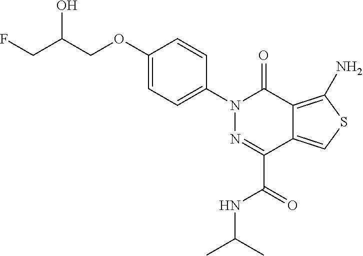 [SMILES: CC(C)NC(=O)c1nn(-c2ccc(OCC(O)CF)cc2)c(=O)c2c(N)scc12] | 100 | − | − |
| 61 | 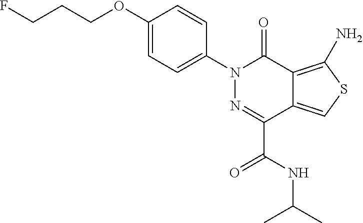 [SMILES: CC(C)NC(=O)c1nn(-c2ccc(OCCCF)cc2)c(=O)c2c(N)scc12] | 100 | − | − |
| 104 | 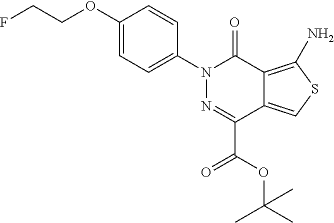 [SMILES: CC(C)(C)OC(=O)c1nn(-c2ccc(OCCF)cc2)c(=O)c2c(N)scc12] | 100 | − | − |

− No staining;
+ weak staining;
++ moderate staining;
+++ intense staining

TABLE 5
Summary of results from in vitro ADME screen

| | | Protein binding | | Liver stability | | | |
|---|---|---|---|---|---|---|---|
| | PAMPA | Human | Rat | Rat (% PCP) | | Human (% PCP) | |
| Com- | Log Pe | plasma | brain | >50% | >50% | >50% | >50% |
| pound | >−6 | <99% | <99% | 5 min | 30 min | 5 min | 30 min |
| PiB | −5.41 | 99.82 | 98.26 | 57.18 | 5.01 | 66.99 | 14.54 |
| ATPZ | −5.24 | 99.02 | 90.94 | 97.06 | 70.19 | 98.9 | 68.29 |
| 38 | −5.02 | 93.58 | 57.2 | 81.7 | 56.4 | 87.2 | 74.4 |
| 14 | −4.89 | 94.78 | 61.7 | 92.8 | 64.0 | 92.1 | 61.3 |
| 105 | −5.10 | 98.0 | 88.8 | 81.29 | 58.8 | 91.5 | 73.2 |
| 58 | −7.52 | 93.2 | 49.4 | 90.03 | 79.3 | 100.9 | 83.4 |
| 90 | −5.51 | 99.16 | 88.84 | 72.67 | 53.0 | 44.62 | 2.57 |
| 56 | −5.16 | 96.28 | 55.2 | 94.88 | 92.06 | 80.98 | 52.10 |
| 81 | −5.12 | 95.02 | 41.07 | 60.12 | 32.54 | 91.63 | 73.10 |

TABLE 6
Results from cold bio-distribution in rat

| | | | | | Clearance |
|---|---|---|---|---|---|
| | Brain uptake % ID/g p.i (min) | | | | ratio |
| Compound | 2 | 10 | 30 | 60 | 2:30 min |
| 38 | 0.28 | 0.081 | 0.021 | 0.011 | 13.3 |
| 106 | 0.014 | 0.001 | 0.004 | 0.001 | 3.5 |
| 99 | 0.31 | 0.092 | 0.013 | 0.001 | 23.8 |

TABLE 7
Results from bio-distribution of radiolabelled compounds in mouse

| | | | | | Clearance |
|---|---|---|---|---|---|
| | Brain uptake % ID/g p.i (min) | | | | ratio |
| Compound | 2 | 10 | 30 | 60 | 2:30 min |
| [¹⁸F]-38 | 6.50 | 3.26 | 2.83 | 2.35 | 2.3 |
| [¹⁸F]-61 | 9.84 | 5.77 | 3.46 | 1.51 | 2.8 |

### REFERENCES

Agdeppa E D, Kepe V, Liu J, Flores-Torres S, Satyamurthy N, Petric A, Cole G M, Small G W, Huang S C, Barrio J R (2001) Binding characteristics of radiofluorinated 6-dialkylamino-2-naphthylethylidene derivatives as positron emission tomography imaging probes for beta-amyloid plaques in Alzheimer's disease. J Neurosci 21:RC189.
Augustinack J C, Schneider A, Mandelkow E M, Hyman B T (2002) Specific tau phosphorylation sites correlate with severity of neuronal cytopathology in Alzheimer's disease. Acta Neuropathol 103:26-35.
Ballatore C, Brunden K R, Piscitelli F, James M J, Crowe A, Yao Y, Hyde E, Trojanowski J Q, Lee V M, Smith A B, 3rd (2010) Discovery of brain-penetrant, orally bioavailable aminothienopyridazine inhibitors of tau aggregation. J Med Chem 53:3739-3747.
Cullen K M, Halliday G M, Cartwright H, Kril J J (1996) Improved selectivity and sensitivity in the visualization of neurofibrillary tangles, plaques and neuropil threads. Neurodegeneration 5:177-187.
Ikonomovic M D, Klunk W E, Abrahamson E E, Mathis C A, Price J C, Tsopelas N D, Lopresti B J, Ziolko S, Bi W, Paljug W R, Debnath M L, Hope C E, Isanski B A, Hamilton R L, DeKosky S T (2008) Post-mortem correlates of in vivo PiB-PET amyloid imaging in a typical case of Alzheimer's disease. Brain: a journal of neurology 131:1630-1645.
Jeganathan S, Hascher A, Chinnathambi S, Biernat J, Mandelkow E M, Mandelkow E (2008) Proline-directed pseudo-phosphorylation at AT8 and PHF1 epitopes induces a compaction of the paperclip folding of Tau and generates a pathological (MC-1) conformation. J Biol Chem 283:32066-32076.
Rosenwald A, Reusche E, Ogomori K, Teichert H M (1993) Comparison of silver stainings and immunohistology for the detection of neurofibrillary tangles and extracellular cerebral amyloid in paraffin sections. Acta Neuropathol 86:182-186.
Thompson P W, Ye L, Morgenstern J L, Sue L, Beach T G, Judd D J, Shipley N J, Libri V, Lockhart A (2009) Interaction of the amyloid imaging tracer FDDNP with hallmark Alzheimer's disease pathologies. J Neurochem 109:623-630.
Uchihara T (2007) Silver diagnosis in neuropathology: principles, practice and revised interpretation. Acta Neuropathol 113:483-499.
Uchihara T, Nakamura A, Yamazaki M, Mori O (2001) Evolution from pretangle neurons to neurofibrillary tangles monitored by thiazin red combined with Gallyas method and double immunofluorescence. Acta Neuropathol 101:535-539.

All patents, journal articles, publications and other documents discussed and/or cited above are hereby incorporated by reference.

1. A compound of Formula I: 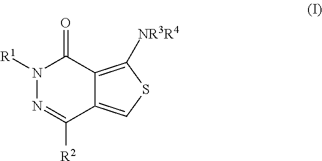
wherein:
R¹ is alkyl or Ar, optionally substituted with at least one alkyl, halogen, hydroxyl, alkoxy, haloalkoxy, acid, ester, amino, nitro, amide, or alkoxyhalo;
R² is independently alkyl, alkynyl, ester, amino, amide, acid, aryl, heteroaryl, aminoalkyl, —C(═O)alkyl, —C(═O)aryl, —C(═O)heteroaryl, —C(═O)heterocycloalkyl, —C(═O)heterocycloalkylAr, —C(═O)(CH₂)ₙhalo, —C(═O)(CH₂)ₙheterocyclyl, or —SO₂Ar, optionally substituted with at least one alkyl, alkylhalo, halogen, nitro, aryl, heteroaryl, or heteroaryl(CH₂)ₙhalo;
R³ and R⁴ are independently hydrogen, alkyl, alkenyl, alkynyl, aryl, heteroaryl;
Ar is an aryl, heteroaryl, cycloalkyl, heterocycloalkyl group;
n is an integer from 0-10; or a radiolabelled derivative thereof.

2. The compound according to claim 1 wherein Ar is selected from the group consisting of:

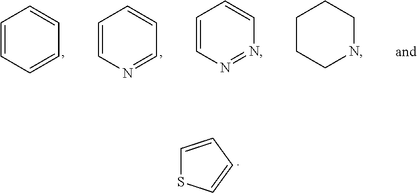

3. (canceled)

4. A compound according to claim 2 having Formula (Ib): 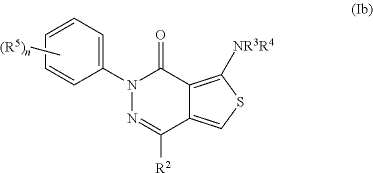
wherein:
R² is independently alkyl, alkynyl, ester, amino, amide, acid, aryl, heteroaryl, aminoalkyl, —C(═O)alkyl, —C(═O)aryl, —C(═O)heteroaryl, —C(═O)heterocycloalkyl, —C(═O)heterocycloalkylAr, —C(═O)(CH₂)ₚhalo, —C(═O)(CH₂)ₚheterocyclyl, or —SO₂Ar, optionally substituted with at least one alkyl, alkylhalo, halogen, nitro, aryl, heteroaryl, or heteroaryl(CH₂)ₚhalo;
R³ and R⁴ are independently hydrogen, alkyl, alkenyl, alkynyl, aryl, or heteroaryl;
Ar is an aryl, heteroaryl, cycloalkyl, or heterocycloalkyl group;
p is an integer from 0-10;
R⁵ is hydrogen, alkyl, halogen, hydroxyl, alkoxy, haloalkoxy, acid, ester, amino, nitro, or amide; and
n is an integer from 0-5; or a radiolabelled derivative thereof.

5. A compound according to claim 4 having Formula (Ic): 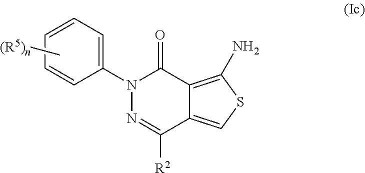
wherein:
R² is independently alkyl, alkynyl, ester, amino, amide, acid, aryl, heteroaryl, aminoalkyl, —C(═O)alkyl, —C(═O)aryl, —C(═O)heteroaryl, —C(═O)heterocycloalkyl, —C(═O)heterocycloalkylAr, —C(═O)(CH₂)ₙhalo, —C(═O)(CH₂)ₙheterocyclyl, or —SO₂Ar, optionally substituted with at least one alkyl, alkylhalo, halogen, nitro, aryl, heteroaryl, or heteroaryl(CH₂)ₙhalo;
R⁵ is hydrogen, alkyl, halogen, hydroxyl, alkoxy, haloalkoxy, acid, ester, amino, nitro, or amide; and
n is an integer from 0-5; or a radiolabelled derivative thereof.

6. A compound according to claim 1 having Formula (Ida), (Idb), or (Idc): 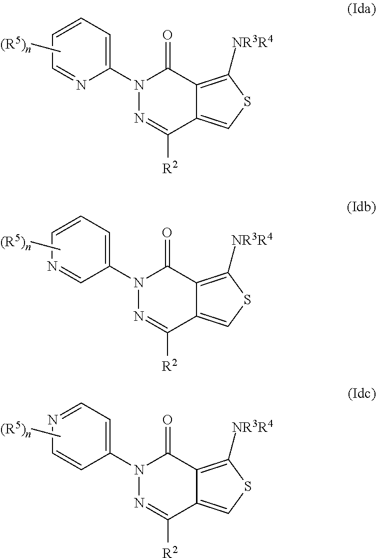
wherein:
R² is independently alkyl, alkynyl, ester, amino, amide, acid, aryl, heteroaryl, aminoalkyl, —C(═O)alkyl, —C(═O)aryl, —C(═O)heteroaryl, —C(═O)heterocycloalkyl, —C(═O)heterocycloalkylAr, —C(═O)(CH₂)ₚhalo, —C(═O)(CH₂)ₚheterocyclyl, or —SO₂Ar, optionally substituted with at least one alkyl, alkylhalo, halogen, nitro, aryl, heteroaryl, or heteroaryl(CH₂)ₚhalo;
R³ and R⁴ are independently hydrogen, alkyl, alkenyl, alkynyl, aryl, or heteroaryl;
R⁵ is hydrogen, alkyl, halogen, hydroxyl, alkoxy, haloalkoxy, acid, ester, amino, nitro, or amide; and
n is an integer from 0-5; or a radiolabelled derivative thereof.

7. A compound according to claim 6 having Formula (Iea), (Ieb) or (Iec): 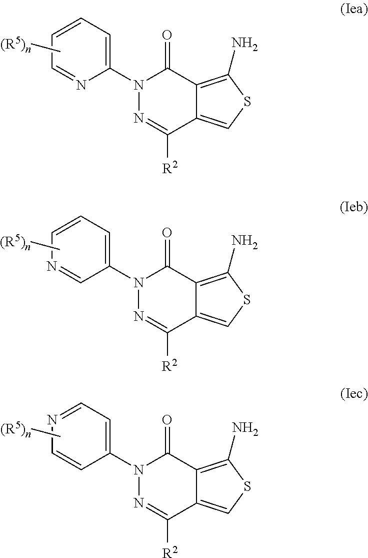
wherein:
R² is independently alkyl, alkynyl, ester, amino, amide, acid, aryl, heteroaryl, aminoalkyl, —C(═O)alkyl, —C(═O)aryl, —C(═O)heteroaryl, —C(═O)heterocycloalkyl, —C(═O)heterocycloalkylAr, —C(═O)(CH₂)ₚhalo, —C(═O)(CH₂)ₚheterocyclyl, or —SO₂Ar, optionally substituted with at least one alkyl, alkylhalo, halogen, nitro, aryl, heteroaryl, or heteroaryl(CH₂)ₚhalo;
R⁵ is hydrogen, alkyl, halogen, hydroxyl, alkoxy, haloalkoxy, acid, ester, amino, nitro, or amide; and
n is an integer from 0-5; or a radiolabelled derivative thereof.

8. A compound of Formula (If): 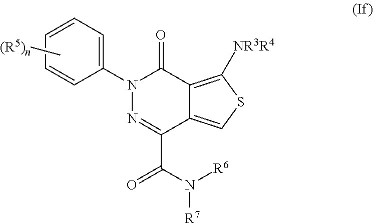
wherein:
R³ and R⁴ are each as defined herein for a compound of Formula (I);
R⁵ is hydrogen, alkyl, halogen, hydroxyl, alkoxy, haloalkoxy, acid, ester, amino, nitro, or amide;
n is an integer from 0-5;
R⁶ and R⁷ are independently hydrogen, alkyl, or alkynyl, or when taken together with the nitrogen to which they are attached form a heteroaryl or heterocycloalkyl optionally substituted with at least one alkyl, alkylhalo, halogen, hydroxyl, nitro, aryl, heterocycloalkyl, heteroaryl, or heteroarylhalo;
or a radiolabelled derivative thereof.

9. A compound of the formula:

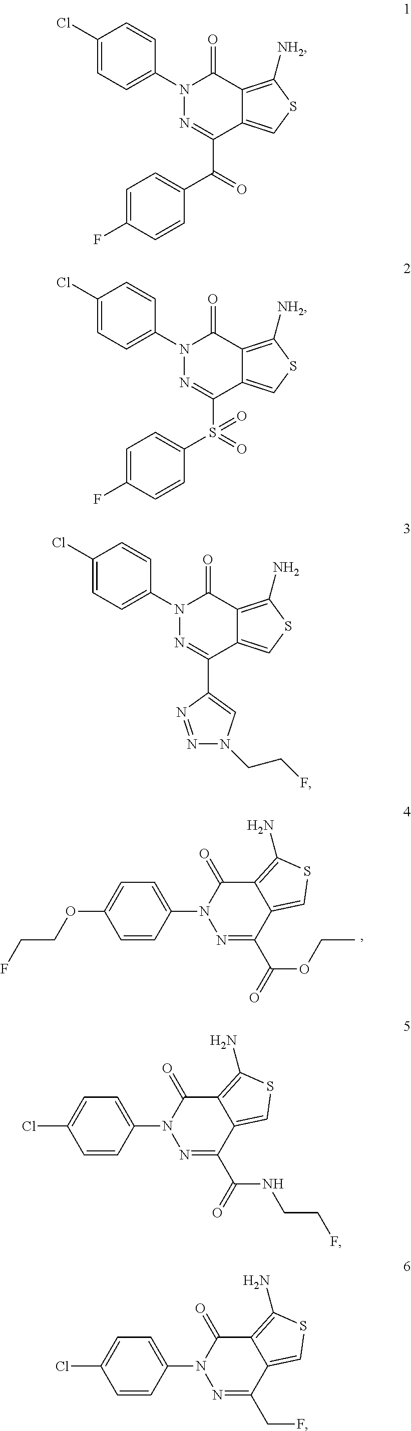 [SMILES: Nc1scc2c(C(=O)c3ccc(F)cc3)nn(-c3ccc(Cl)cc3)c(=O)c12 ; Nc1scc2c(S(=O)(=O)c3ccc(F)cc3)nn(-c3ccc(Cl)cc3)c(=O)c12 ; Nc1scc2c(-c3cn(CCF)nn3)nn(-c3ccc(Cl)cc3)c(=O)c12 ; CCOC(=O)c1nn(-c2ccc(OCCF)cc2)c(=O)c2c(N)scc12 ; Nc1scc2c(C(=O)NCCF)nn(-c3ccc(Cl)cc3)c(=O)c12 ; Nc1scc2c(CF)nn(-c3ccc(Cl)cc3)c(=O)c12]

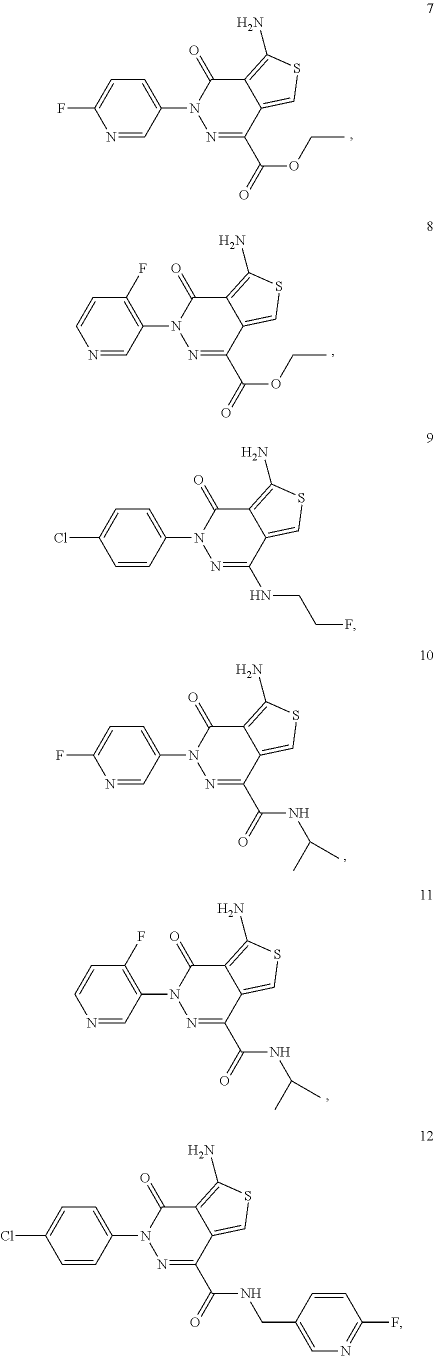

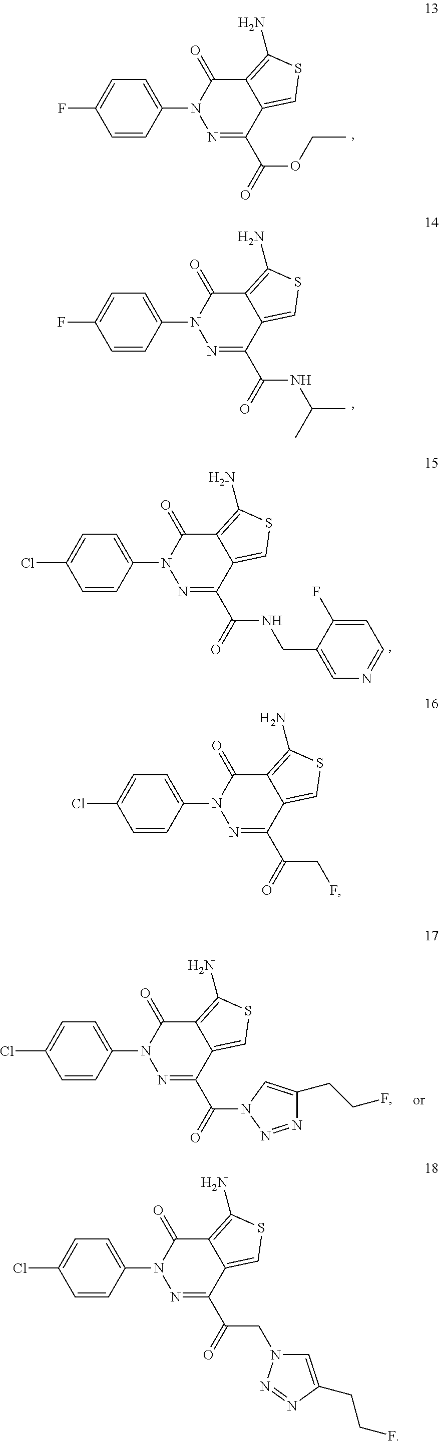

10. (canceled)

11. A compound of the formula of:

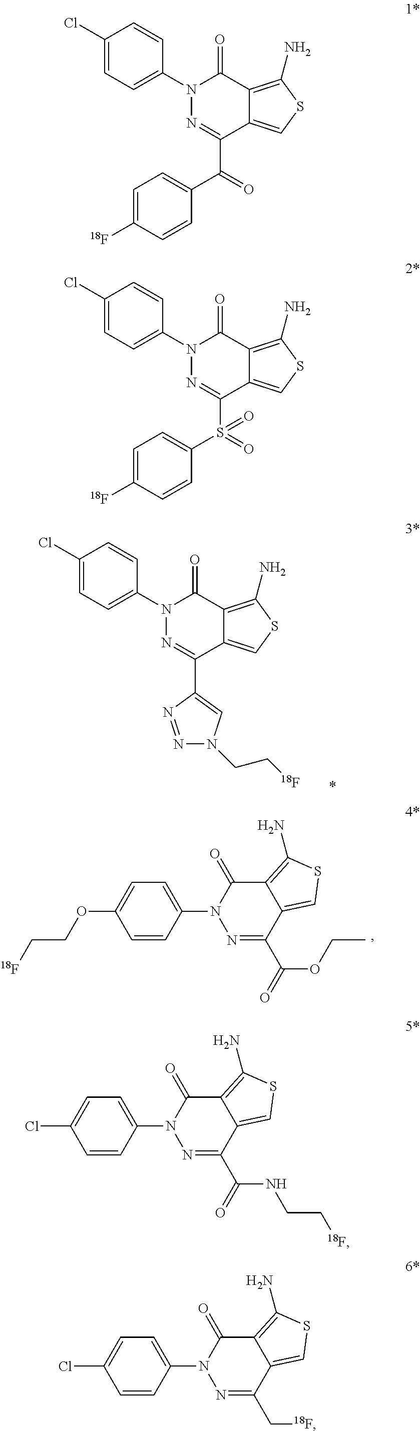

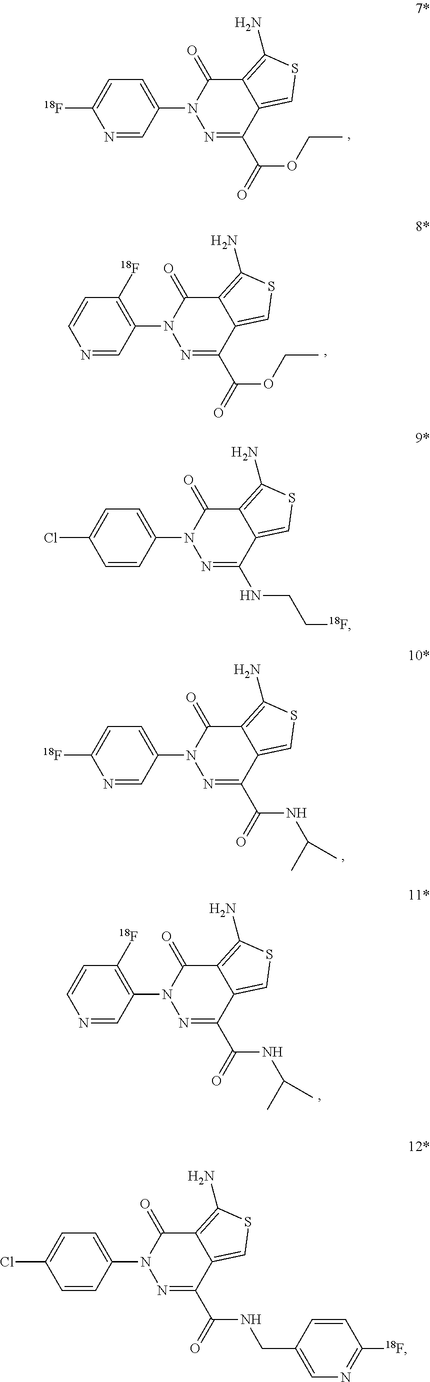

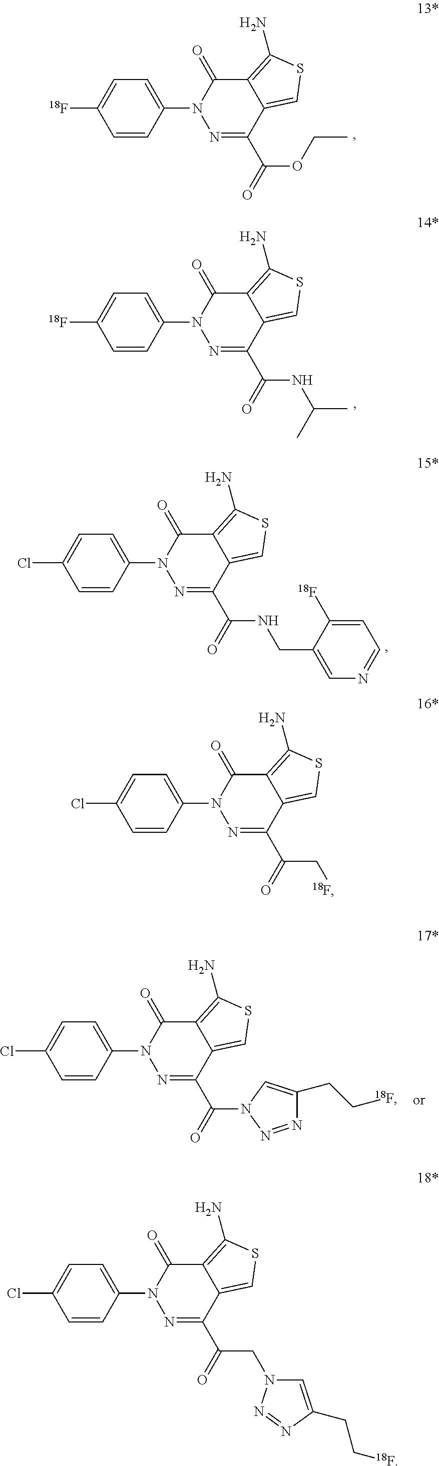

12. (canceled)

13. (canceled)

14. (canceled)

15. (canceled)

16. (canceled)

17. A composition comprising a compound according to claim 1 and a pharmaceutically acceptable carrier or excipient.

18. (canceled)

19. A method of imaging using a compound according to claim 1 or a pharmaceutical composition thereof.

20. A method of detecting tau aggregates in vitro and/or in vivo using a compound according to claim 1 or a pharmaceutical composition thereof.

21. (canceled)

22. (canceled)

23. (canceled)
